(19) United States
(12) Patent Application Publication
(10) Pub. No.: US 2003/0130264 A1
(43) Pub. Date: Jul. 10, 2003

(54) Methods of using pyrimidine-based antiviral agents

(75) Inventor: Juan C. Jaen, Burlingame, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Tularik Inc., So. San Francisco, CA (US)

(21) Appl. No.: 10/078,246

(22) Filed: Feb. 14, 2002

Related U.S. Application Data
(60) Provisional application No. 60/269,778, filed on Feb. 16, 2001.

(51) Int. Cl. A61K 31/55; A61K 31/513; A61K 31/506

(52) U.S. Cl. 514/217.06; 514/269; 514/275

(57) ABSTRACT

The invention provides novel methods of using substituted pyrimidine compounds and compositions for the treatment or prevention of diseases associated with CMV infection. In particular, the present invention provides methods for the treatment or prevention of cardiovascular diseases and organ transplant rejection.

### CROSS-REFERENCE TO RELATED APPLICATIONS

This application claims the benefit of U.S. Application Serial No. 60/269,778, filed Feb. 16, 2001 and is related to U.S. application Ser. No. 09/757,291, filed Jan. 8, 2001, which claims the benefit of U.S. Application Serial No. 60/176,000, filed Jan. 12, 2000; and is related to U.S. application Ser. No. 09/249,641, filed Feb. 12, 1999, which is a continuation-in-part of U.S. Application Serial No. 60/075,005, filed Feb. 17, 1998, the disclosures of which are incorporated by reference herein.

### FIELD OF THE INVENTION

The field of the invention relates to methods of using substituted pyrimidine compounds to treat and suppress diseases associated with human cytomegalovirus infection. The subject methods are particularly useful in treating and suppressing cardiovascular disease and organ transplant rejection associated with human cytomegalovirus infection.

### BACKGROUND OF THE INVENTION

Cytomegalovirus (CMV) is a member of the herpes virus family. Other well-known members of the herpes virus family include, for example, herpes simplex virus, types I and II, Epstein-Barr virus and varicella zoster virus. These viruses are related taxonomically, but each manifests in a clinically distinct manner. In the case of CMV, medical conditions arising from congenital infection include jaundice, respiratory distress and convulsive seizures which may result in mental retardation, neurologic disability or death. Infection in adults is frequently asymptomatic, but may manifest as mononucleosis, hepatitis, pneumonitis or retinitis, particularly in immunocompromised patients such as AIDS sufferers, chemotherapy patients, and organ transplant patients undergoing tissue rejection therapy.

A variety of drugs have been developed to treat herpes virus infections, including naturally occurring proteins and synthetic nucleoside analogs. For example, the natural antiviral protein interferon has been used in the treatment of herpes virus infections, as have the nucleoside analogs cytosine-arabinoside, adenine-arabinoside, iodoxyuridine and acyclovir, which is presently the treatment of choice for herpes simplex type II infection.

Unfortunately, drugs such as acyclovir that have proven sufficiently effective to treat infection by certain herpes viruses are not sufficiently effective to treat CMV. Additionally, drugs currently used to treat CMV infection, such as 9-((1,3-dihydroxy-2-propoxy)methyl)guanidine (ganciclovir, DHPG), which inhibits viral DNA synthesis, phosphonoformic acid (foscarnet), cidofovir and the antisense agent fomivirsen, lack the acceptable side effect and safety profiles of the drugs approved for treatment of other herpes viruses. Moreover, such drugs are ineffective to treat certain strains of CMV that have acquired drug resistance. Thus, despite advances in the development of anti-herpes virus drugs, there remains a need for therapeutic agents effective in treating CMV infection with an increased safety margin.

### SUMMARY OF THE INVENTION

The present invention provides methods of using substituted pyrimidine compounds and compositions for treating or preventing diseases, particularly diseases associated with CMV infection. In particular, the present invention provides methods for treating or preventing cardiovascular disease, including, but not limited to, atherosclerosis and restenosis, and organ transplant rejection associated with CMV infection.

U.S. application Ser. No. 09/249,641 and PCT Publication No. WO99/41253 describe pyrimidine derivatives for the treatment of viral infections, and U.S. Application Serial No. 60/176,000 describes certain salts of pyrimidine derivatives which have properties suitable for clinical use for the treatment of viral infections. The present invention contemplates the use of these and other pyrimidine derivatives in the described methods.

The compounds of the invention have the general formula (I):

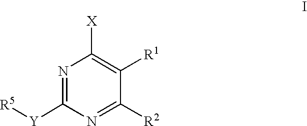

in which X represents —NR³R⁴, —OR³, —SR³, aryl, alkyl or arylalkyl. The letter Y represents a covalent bond, —N(R⁶)—, —O—, —S—, —C(═O)— or an alkylene group. R¹ and R² are independently selected from hydrogen, alkyl, —O-alkyl, —S-alkyl, aryl, arylalkyl, —O-aryl, —S-aryl, —NO₂, —NR⁷R⁸, —C(O)R⁹, —CO₂R¹⁰, —C(O)NR⁷R⁸—N(R⁷)C(O)R⁹, —N(R⁷)CO₂R¹¹, —N(R⁹)C(O)NR⁷R⁸, —S(O)ₘNR⁷R⁸, —S(O)ₙR⁹, —CN, halogen, and —N(R⁷)S(O)ₘR¹¹. The groups R³ and R⁴ are independently selected from hydrogen, alkyl, aryl or arylalkyl, or, when X is —NR³R⁴, R³ and R⁴ are combined to form a 5-, 6- or 7-membered aromatic or nonaromatic ring containing from one to three heteroatoms in the ring. R⁵ and R⁶ are independently hydrogen, alkyl, aryl or arylalkyl. R⁷ and R⁸ are each independently hydrogen, alkyl, aryl or arylalkyl, or, when attached to the same nitrogen atom can be combined with the nitrogen atom to form a 4-, 5-, 6-, 7- or 8-membered ring containing from one to three heteroatoms in the ring. R⁹ and R¹⁰ are independently selected from hydrogen, alkyl, aryl and arylalkyl. R¹¹ is selected from alkyl, aryl and arylalkyl. The subscript m is an integer of from 1 to 2 and the subscript n is an integer of from 1 to 3.

In addition to the above descriptions of R¹ to R¹¹, the formula above is meant to represent a number of compounds in which a second ring is fused to the pyrimidine ring. For example, R¹ can be joined to R², R¹ can be joined to R³, R³ can be joined to N³(the nitrogen atom at the 3-position of the pyrimidine ring), R⁵ can be joined to N³, R⁵ can be joined to N¹(the nitrogen atom at the 1-position of the pyrimidine ring) or R² can be joined to N¹ to form a fused 5-, 6-, or 7-membered ring.

Unless otherwise indicated, the compounds provided in the above formula are meant to include pharmaceutically acceptable salts and prodrugs thereof.

Other objects, features and advantages of the present invention will become apparent to those skilled in the art from the following description and claims.

### BRIEF DESCRIPTION OF THE DRAWINGS

FIG. 1 provides the structures of exemplary compounds of formula IIa.

FIG. 2 provides the structures of exemplary compounds of formula IIb.

FIG. 3 provides the structures of exemplary compounds of formula IIc.

FIG. 4 provides the structures of exemplary compounds of formula IId.

FIG. 5 provides the structures of exemplary compounds of formula IIe.

FIG. 6 provides the structures of exemplary compounds of formula IIf.

FIGS. 7-16 provide synthesis schemes for exemplary compounds of formulae IIa-IIf and also selected transformations for functional groups present on the compounds.

FIG. 17 provides the structures of compounds used in radiolabeling studies.

FIG. 18 shows the binding of radiolabeled compounds to viral specific protein. Tritiated compounds bind covalently to a 110-kD viral specific protein that appears at 48 h post infection. Phosphoimager generated images of radiolabeled infected cell proteins separated by SDS polyacrylamide gel electrophoresis are shown. Panel A: Time course analysis (24-96 h) of radiolabeled proteins from HCMV infected or uninfected cells in the presence of 0.1 μM (³H)-d. Panel B: Pattern of radiolabeling in uninfected (UI) cells and in cells infected for 72 h with HCMV (I) and treated with either 0.01 μM (³H)-d or 0.02 μM (³H)-17. Protein X, the 110-kD viral specific protein, is indicated by the arrows. Panel C: Pattern of radiolabeling of cytoplasmic and nuclear extracts prepared from HFF cells infected for 96 h with wild type HCMV (rHCMVLUC) and labeled with either 0.5 μM (³H)-d or 0.5 μM (³H)-25.3. Varying amounts (5-50 μL) of the (³H)-d labeled or M (³H)-25.3 labeled extracts were analyzed. X and the arrow indicate protein X, the 110-kD viral specific protein.

FIG. 19 shows the reaction of UL70 peptide antibodies with viral specific protein. Protein X, the molecular target of (³H)-d is a viral specific nuclear protein that is immunoprecipitated with antibodies to UL70 and UL105. Panel A: A phosphoimager generated image of a Western blot with antiserum generated to a 30-amino acid peptide from the predicted amino acid sequence of the UL70 open reading frame. Extracts from High Five cells infected with a control baculovirus (control lysate) or baculovirus expressing the CMV UL70 protein lacking the first N-terminal 100 amino acids (ΔNUL70 lysate) were subjected to SDS polyacrylamide electrophoresis in 4-20% gradient gels. The gel-separated proteins were then transferred to nitrocellulose and probed with either preimmune serum (Panel 1) or antiserum raised to the UL70 peptide (Panel 2). At a 1:10,000 dilution of UL70 antiserum (Panel 2), a strong signal at about 85 kD was observed only in extracts from cells infected with baculovirus that expresses the truncated UL70 protein (Ab, antibody). Panel B: Uninfected cells and cells infected with rHCMVLUC at an MOI of 5 pfu/cell for 72 h were treated with (³H)-d. Extracts from theses cells were subjected to SDS polyacrylamide electrophoresis in 4-20% gradient gels. The gel-separated proteins were then transferred to nitrocellulose, exposed to Fuji tritium detection plates, and analyzed with a phosphoimager. A (³H)-d labeled, 110-kD protein (protein X), was detected by phosphoimaging and is shown in Panel 2. The same filter was then probed with UL70 antiserum, and the Western blot is shown in Panel 1. UL70 can be detected in infected cells, and the UL70 antibody signal comigrates exactly with the (³H)-d labeled, 110-kD viral specific protein X (Ab, antibody). Panel C: Phosphoimager generated image of cytoplasmic and nuclear extracts prepared from HFF cells infected with wild type HCMV (rHCMVLUC) and labeled with (³H)-d. Varying amounts (5-60 μL) of the (³H)-d labeled extracts were subjected to SDS polyacrylamide gel electrophoresis in 10% gels. The gel-separated proteins were transferred too nitrocellulose filters and exposed to Fuji plates for the detection of tritium. Panel 1 shows a titration of the cytoplasmic extract from 8×10⁸ HFF cells; Panel 2 shows a titration of the corresponding nuclear extract from the same 8×10⁸ cells. A single nuclear specific (³H)-d labeled protein is detected at 110 kD (Panel 2); Panel 3 (IPs) shows phosphoimager generated images of the same nuclear extracts shown in Panel 2 immunoprecipitated with UL70 specific antibodies (70 ab), UL105 specific antibodies (105 ab) or UL70 preimmune serum (pis). The arrow identifies the protein X (UL70 primase) at 110 kD.

FIG. 20 shows the amino acid sequence of the Towne strain (HCMV) UL70 open reading frame showing the three point mutations identified in 1-resistant virus. The positions and nature of the point mutations contained in the UL70 protein of the 1-resistant virus are indicated. The virus contains three single base pair mutations. Valine 511 is mutated to isoleucine by a G to A change at the first base of the codon. Proline 571 is mutated to a serine by a C to A change at the first base of the codon. Isoleucine 692 is mutated to a phenylalanine by an A to T change at the first base of the codon. The boxed regions indicate the five domains in herpesvirus primases. The asterisk at residue 570 indicates a cysteine residue that is a potential site of covalent modification by the drug.

### DETAILED DESCRIPTION OF THE INVENTION

### Abbreviations and Definitions

The abbreviations used herein are conventional, unless otherwise defined.

The terms “treat”, “treating” and “treatment” refer to a method of alleviating or abrogating a disease and/or its attendant symptoms.

The terms “prevent”, “preventing” and “prevention” refer to a method of barring a subject from acquiring a disease. As used herein, “prevent”, “preventing” and “prevention” also include reducing a subject's risk of acquiring a disease.

The term “disease associated with CMV infection” is meant to include any disease, disorder, dysfunction and the like, in which CMV infection contributes, directly or indirectly, to the pathogenesis thereof. For example, CMV infection may produce immunologic responses that cause endothelial injury and precipitate atherogenesis. Exemplary diseases associated with CMV infection include, but are not limited to, cardiovascular disease, such as atherosclerosis and restenosis, organ transplant associated atherosclerosis and organ transplant rejection.

The term “CMV infection” refers to the invasion and replication of cytomegalovirus (CMV) in cells or tissues. CMV infection may be determined by measuring CMV antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. CMV may infect an organ, e.g., kidney, liver, heart, lung, eye and brain, and cause, e.g., nephritis, hepatitis, myocarditis, retinitis and encephalitis, respectively.

The term “therapeutically effective amount” refers to that amount of the compound being administered sufficient to prevent development of or alleviate to some extent one or more of the symptoms of the disease being treated.

“Cardiovascular disease”, as used herein, refers disorders of the heart and/or blood vessels and includes, but is not limited to, aneurysm, atherosclerosis, cardiomyopathy, congestive heart failure, coronary artery disease, hypertension, ischemia/reperfusion, restenosis and vascular stenosis. Excess lipid accumulation in the arterial walls, which forms plaques that inhibit blood flow and promote clot formation, is the primary cause of cardiovascular disease. In vascular grafts and transplanted organs, cardiovascular disease is often accelerated.

“Organ transplant rejection”, as used herein, refers to a process leading to the destruction or detachment of a transplanted organ, such as a heart, kidney, lung, liver, pancreas, bowel, bone marrow and the like, or a combination thereof, e.g., heart-lung, or the destruction or damage of certain host organs. Rejection is caused by reaction of the host's immune cells to the transplanted organ(s) or bone marrow as foreign, and/or reaction of the donor's immune cells to the recipient as foreign. Rejection may be acute or chronic. The transplanted organ or bone marrow may be an allograft, i.e., from a genetically non-identical member of the same species, or a xenograft, i.e., from a member of different species, e.g., a porcine heart valve.

The term “immunocompromised condition” refers to any condition in which the subject has decreased immune function relative to normal. Immunocompromised conditions include acquired conditions and hereditary conditions.

The term “electrophilic moiety” refers to a chemical group that is electron deficient and is reactive with chemical groups having an excess of electrons, as commonly understood in the art. Exemplary electrophilic moieties include, but are not limited to, isothiocyanate, maleimide, haloacetamide, vinylsulfone, benzylic halide, electron-deficient aromatic rings, such as nitro-substituted pyrimidine rings, and the like.

The term “modulate” refers to the ability of a compound to increase or decrease the catalytic activity of a primase. A modulator preferably activates the catalytic activity of a primase, more preferably activates or inhibits the catalytic activity of a primase depending on the concentration of the compound exposed to the primase, or most preferably inhibits the catalytic activity of a primase.

The term “modify” refers to the act of altering or altering in part, e.g., the structure of a molecule, e.g., a protein. Modification may be covalent or noncovalent, and includes, but is not limited to, aggregation, association, substitution, conjugation and/or elimination of a chemical group. Modification may alter the function or other properties (e.g., chemical, physical) of the molecule.

The term “alkyl,” by itself or as part of another substituent, means, unless otherwise stated, a straight or branched chain or cyclic hydrocarbon radical or combinations thereof, which may be filly saturated, mono- or polyunsaturated and can include di- and multi-radicals, having the number of carbon atoms designated (i.e. C₁-C₈ means one to eight carbons). Examples of saturated hydrocarbon radicals include straight or branched chain groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. Other saturated hydrocarbon radicals include cyclopropylmethyl, cyclohexylmethyl and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined below as heteroalkyl, alkylene, heteroalkylene, cycloalkyl and heterocycloalkyl. Typically, an alkyl group will have from 1 to 24 carbon atoms, with those groups having 10 or fewer carbon atoms being preferred in the present invention. The term “alkylene” by itself or as part of another substituent means a divalent radical derived from an alkane, as exemplified by —CH₂CH₂CH₂CH₂—. A “lower alkyl” or “lower alkylene” is a shorter chain alkyl or alkylene group, generally having eight or fewer carbon atoms. Unless otherwise indicated, the alkyl groups can be unsubstituted or substituted by the substituents indicated below.

The term “heteroalkyl,” by itself or in combination with another term, means, unless otherwise stated, a stable straight or branched chain radical consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—.

The terms “cycloalkyl” and “heterocycloalkyl”, by themselves or in combination with other terms, represent, unless otherwise stated, cyclic versions of “alkyl” and “heteroalkyl”, respectively. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “fluoroalkyl,” are meant to include monofluoroalkyl and polyfluoroalkyl. More particularly, the term “fluoroalkyl” also includes perfluoroalkyl, in which each hydrogen present in an alkyl group has been replaced by a fluorine.

The term “aryl,” employed alone or in combination with other terms (e.g., aryloxy, arylthioxy, arylalkyl) means, unless otherwise stated, an aromatic substituent which can be a single ring or multiple rings (up to three rings) which are fused together or linked covalently. The rings may each contain from zero to four heteroatoms selected from N, O, and S, wherein the nitrogen and sulfur atoms are optionally oxidized, and the nitrogen atom(s) are optionally quaternized. Non-limiting examples of aryl groups include phenyl, 1-naphthyl, 2-naphthyl, biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl ring systems are selected from the group of acceptable substituents described below.

As used herein, the term “bicyclic fused aryl-cycloalkyl” refers to those groups in which an aryl ring (or rings) is fused to a cycloalkyl group (including cycloheteroalkyl groups). The group can be attached to the remainder of the molecule through either an available valence on the aryl portion of the group, or an available valence on the cycloalkyl portion of the group. Examples of such bicyclic fused aryl-cycloalkyl groups are: indanyl, benzotetrahydrofuranyl, benzotetrahydropyranyl and 1,2,3,4-tetrahydronaphthyl.

Each of the above terms (e.g., “alkyl” and “aryl” and “bicyclic fused aryl-cycloalkyl”) will typically include both substituted and unsubstituted forms of the indicated radical. Preferred substituents for each type of radical are provided below. In the case of radicals containing both aryl (including heteroaryl) and alkyl (including, for example, heteroalkyl, cycloalkyl, and cycloheteroalkyl) portions, each of the portions can be substituted as indicated.

Substituents for the alkyl groups (including those groups often referred to as alkenyl, heteroalkyl, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halo, —SiR′R″R′″, —OC(O)R′, —CO₂R′, —CONR′R″, —OC(O)NR′R″, —NR″C(O)R′, —NR″—C(O)—OR′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2N+1), where N is the total number of carbon atoms in such radical. R′, R″ and R′″ each independently refer to a hydrogen or C1-C10 alkyl group. Preferably, a substituted alkyl group will have from one to six independently selected substituents. More preferably, a substituted alkyl group will have from one to four independently selected substituents. Nevertheless, certain substituted alkyl groups (e.g., perfluoroalkyl) will have a full 2N+1 substituents (where N is the number of carbon atoms in a saturated alkyl group). Examples of substituted alkyl groups include: —C(O)—CH₃, —C(O)CH₂OH, —CH₂—CH(CO₂H)—NH₂ and —Si(CH₃)₂—CH₂—C(O)—NH₂.

Similarly, substituents for the aryl groups are varied and are selected from: -halo, —OR′, —OC(O)R′, —NR′R″, —SR′, —R′, —CN, —NO₂, —CO₂R′, —CONR′R″, —OC(O)NR′R″, —NR″C(O)R′, —NR″—C(O)—OR′, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′ and R″ are independently selected from hydrogen, (C₁-C₈)alkyl, aryl, aryl-(C₁-C₄)alkyl, and aryloxy-(C₁-C₄)alkyl.

Two of the substituents on adjacent atoms of the aryl ring may optionally be replaced with a substituent of the formula —T—C(O)—(CH₂)ₛ—U—, wherein T and U are independently —NH—, —O—, —CH₂— or a single bond, and the subscript s is an integer of from 0 to 2. Alternatively, two of the substituents on adjacent atoms of the aryl ring may optionally be replaced with a substituent of the formula —A—(CH₂)ₚ—B—, wherein A and B are independently —CH₂—, —O—, —NH—, —S—, —S(O)—, —S(O)₂—, —S(O)₂NR′— or a single bond, and p is an integer of from 1 to 3. One or more of the single bonds of the new ring so formed may optionally be replaced with a double bond. Alternatively, two of the substituents on adjacent atoms of the aryl ring may optionally be replaced with a substituent of the formula —(CH₂)_{q}—Z—(CH₂)ᵣ—, where q and r are independently integers of from 1 to 3, and Z is —O—, —NR′—, —S—, —S(O)—, —S(O)₂—, or —S(O)₂NR′—. The substituent R′ in —NR′— and —S(O)₂NR′— is selected from hydrogen or (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, salicylic, tartaric, methanesulfonic, and the like. Also included are salts of amino acids such as arginate and the like, and salts of organic acids like glucuronic or galactunoric acids and the like (see, for example, Berge, et al. (1977) J. Pharm. Sci., 66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### General

A number of studies have demonstrated an association between CMV infection and the development of cardiovascular disease, in particular, atherosclerosis and restenosis, which share the same pathology of cardiovascular endothelial injury. Atherosclerosis, or the progressive narrowing and hardening of the arteries over time due to injury or dysfunction of endothelial and/or smooth muscle cells. In response to such injury or dysfunction, lipid accumulation and plaque formation occurs, preceded and accompanied by inflammation. The plaques formed can inhibit blood flow and promote clot formation, ultimately causing heart attacks, stroke and claudication. Restenosis is the re-narrowing and/or hardening of a blood vessel that can develop following a procedure, such as balloon angioplasty, aimed at opening the blood vessel. For example, CMV DNA has been detected in atherosclerotic lesions (Melnick et al. (1993) Eur. Heart J. 14(Suppl. K):30-38 and Horvath et al. (2000) J. Clin. Virol. 16:17-24) and restenotic lesions (Speir et al. (1994) Science 265:391-394 and Zhou et al. (1996) New Engl. J. Med. 335:624-630). Also, there is evidence that human CMV increases modified LDL uptake and scavenger receptor mRNA expression in vascular smooth muscle cells (Zhou et al. (1996) J. Clin. Invest. 98:2129-2138). CMV infection has been shown to increase the neointimal response to vascular injury without consistent evidence of direct infection of the vascular wall (Zhou et al. (1999) Circulation 100:1569-1575). More recently, the effects of chronic non-vascular CMV infection on the neointimal response to experimental vascular injury has been demonstrated (Zhou et al. (2000) Cardiovasc. Res. 45:1019-1025).

CMV infection has also been associated with graft atherosclerosis and rejection in transplant recipients (see, e.g., Grattan et al. (1989) JAMA 261:3561-3566). For example, CMV infection is associated with the development of accelerated arteriosclerosis in cardiac allografts (Koskinen et al. (1996) Clin. Transplant. 10(6 Pt 1):487-493); bronchiolitis obliterans in lung allografts (Bando et al. (1995) J. Thorac. Cardiovasc. Surg. 110:4-14); hepatic artery thrombosis (Madalosso et al. (1998) Transplantation 66(3):294-297) and transplant renal artery stenosis (Pouria et al. (1998) Q. J. Med. 91:185-189). Evidence suggests that prevention or therapy of CMV infection could increase the chances of graft survival in transplant recipients.

### Embodiments of the Invention

### Compounds

In one aspect, the present invention provides methods of using compounds of general formula (I):

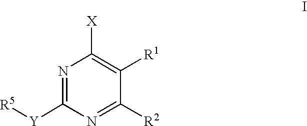

in which X represents —NR³R⁴, —OR³, —SR³, aryl, alkyl or arylalkyl. The letter Y represents a covalent bond, —N(R⁶)—, —O—, —S—, —C(═O)— or an alkylene radical. Preferably, Y is —N(R⁶)— or —O—, in which R⁶ is as defined below. More preferably, Y is —N(R⁶)—. For those embodiments in which Y is an alkylene radical, the alkylene radical will typically have from 1 to 8 carbon atoms in the chain, with alkylene groups having from 1 to 3 carbon atoms being preferred.

R¹ and R² are independently selected from hydrogen, alkyl, —O-alkyl, —S-alkyl, aryl, arylalkyl, —O-aryl, —S-aryl, —NO₂, —NR⁷R⁸, —C(O)R⁹, —CO₂R¹⁰, —C(O)NR⁷R⁸—N(R⁷)C(O)R⁹, —N(R⁷)CO₂R¹¹, —N(⁹)C(O)NR⁷R⁸, —S(O)ₘNR⁷R⁸, —S(O)ₙR⁹, —CN, halogen, or —N(R⁷)S(O)ₘR¹¹, in which R⁷, R⁸, R⁹, R¹⁰ and R¹¹ are as defined below.

In one group of preferred embodiments, R¹ is an electron-withdrawing group and R² is an electron-donating group. Within this group of embodiments, R′ is preferably —NO₂, —S(O)ₘNR⁷R⁸, —S(O)ₙR⁹, —CN, halogen, fluoroalkyl, —C(O)R⁹, —CO₂R¹⁰ or —C(O)NR⁷R⁸. More preferably, R¹ is —CF₃, —NO₂, —CN, —S(O)ₘNR⁷R⁸, or —CO₂R¹⁰, with —NO₂ being the most preferred. The R² group is preferably hydrogen, lower alkyl, —O-alkyl, —S-alkyl, aryl, arylalkyl, —O-aryl or —S-aryl. More preferably, R² will be methyl, ethyl, n-propyl, isopropyl, methoxy, ethoxy, propoxy, methoxymethyl, methylthio, ethylthio or propylthio.

In another group of preferred embodiments, R¹ is an electron-donating group and R² is an electron-withdrawing group. Within this group of embodiments, R¹ is preferably hydrogen, lower alkyl, —O-alkyl, —S-alkyl, aryl, arylalkyl, —O-aryl or —S-aryl. More preferably, R¹ is methyl, ethyl, n-propyl, isopropyl, methoxy, ethoxy, propoxy, methylthio, ethylthio or propylthio. The R² group is preferably —NO₂, —S(O)ₘNR⁷R⁸, —S(O)ₙR⁹, —CN, halogen, fluoroalkyl, —C(O)R⁹, —CO₂R¹⁰ or —C(O)NR⁷R⁸. More preferably, R² is —CF₃, —NO₂, —CN, —S(O)ₘNR⁷R⁸ or —CO₂R¹⁰, with —NO₂ being the most preferred.

The groups R³ and R⁴ are independently hydrogen, alkyl, aryl or arylalkyl, or, combined to form, a 5-, 6- or 7-membered ring containing from one to three heteroatoms in the ring. In one group of preferred embodiments, R³ and R⁴ are combined to form a 5- or 6-membered ring. The rings defined by R³ and R⁴ and the nitrogen atom can be saturated, unsaturated or aromatic, and can contain additional heteroatoms. Examples of suitable rings include: pyrrolidine, pyrrole, pyrazole, imidazole, imidazoline, thiazoline, piperidine, morpholine, and the like. In certain preferred embodiments, R³ and R⁴ are combined to form a 5-membered ring containing two nitrogen atoms, preferably an imidazole ring, and most preferably a 2-alkylimidazole ring or a 5-alkylimidazole ring. Particularly preferred X groups are 2-methylimidazol-1yl, 2,4-dimethylimidazol-1yl, 2-ethylimidazol-1yl, 2-propylimidazol-1yl, 2-isopropylimidazol-1yl and 5-methylimidazol-1yl.

The R⁵ group is an alkyl, aryl, arylalkyl or bicyclic fused aryl-cycloalkyl group. Preferred alkyl groups are those having from one to eight carbon atoms, either substituted or unsubstituted. Preferred aryl groups include substituted or unsubstituted phenyl, pyridyl, or naphthyl. Preferred arylalkyl groups include substituted and unsubstituted benzyl, phenethyl, pyridylmethyl and pyridylethyl. Particularly preferred R⁵ groups are phenyl, 4-halophenyl, benzyl, n-butyl, propionyl, acetyl and methyl. When Y is —N(R⁶)—, other preferred R⁵ groups are those in which R⁵ is combined with R⁶ to form a nonaromatic ring, preferably a include substituted or unsubstituted 1-piperidinyl ring, a substituted or unsubstituted 4-morpholinyl ring or a substituted or unsubstituted 1-pyrrolidinyl ring.

Still other preferred R⁵ groups (including some of the preferred fused bicyclic aryl-cycloalkyl groups) are selected from:

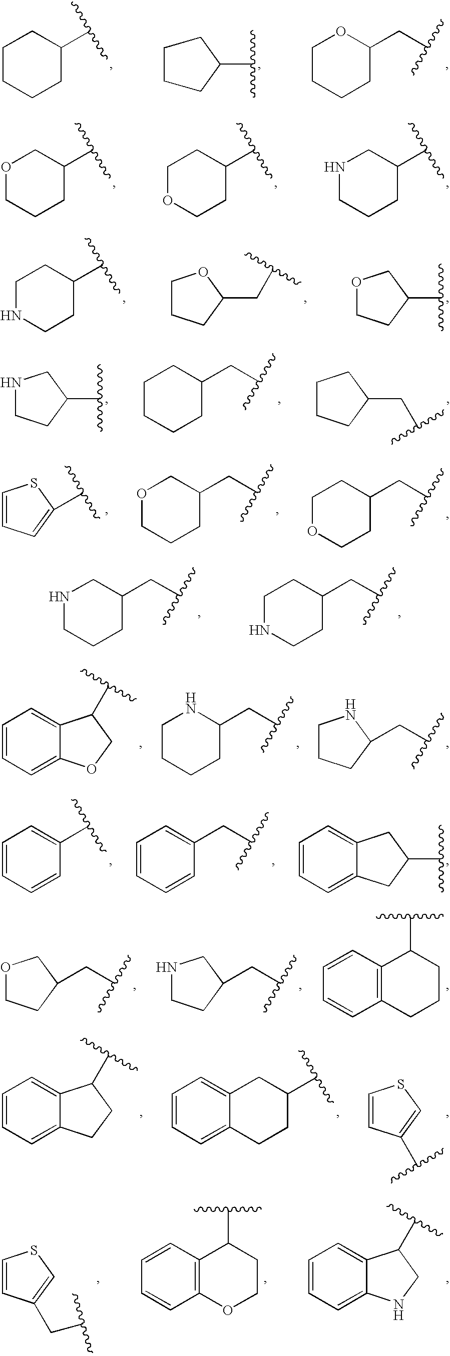

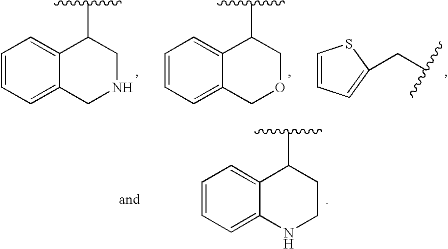

In one group of particularly preferred embodiments, R⁵ is a radical selected from the group consisting of:

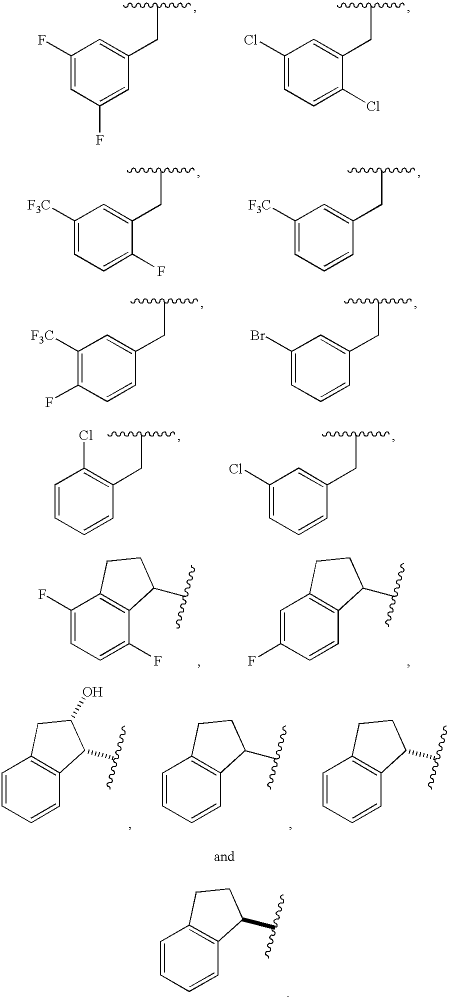

In another group of particularly preferred embodiments, R⁵ is a radical selected from the group consisting of:

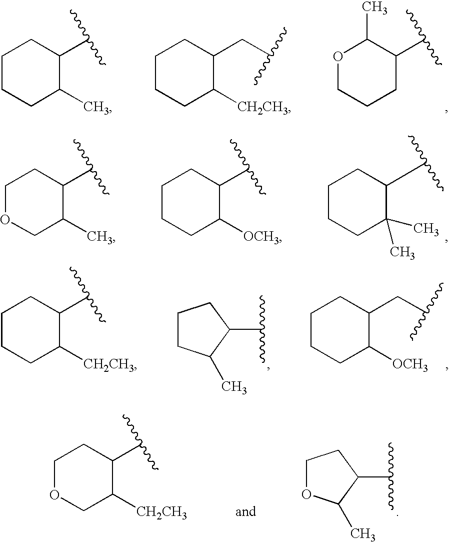

The above group of radicals is meant to include those radicals having a mixture of stereochemistry as well as pure isomers and enantiomers (those having less than about 5% of another diastereomer or enantiomer, more preferably less than about 2% of another isomer, and most preferably less than about 1% of another isomer).The R⁶ group is typically hydrogen, alkyl, aryl or arylalkyl. Preferably, R⁶ is hydrogen, a lower alkyl group having from one to three carbon atoms, a phenyl ring or a phenylalkyl group, such as, for example, a benzyl or a phenethyl group.

In yet another group of preferred embodiments, Y is —N(R⁶)— and R⁵ is combined with R⁶ to form a group selected from the group consisting of:

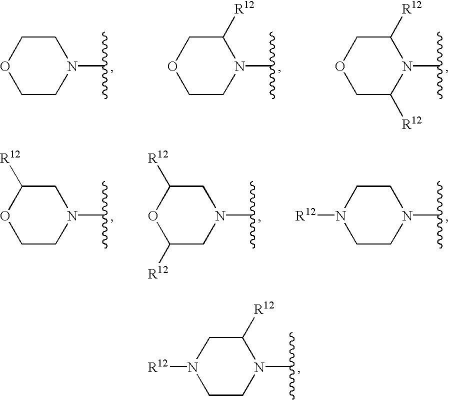

and the like.

R⁷ and R⁸ are each independently hydrogen, alkyl, aryl or arylalkyl, or, combined to form a 4-, 5-, 6-, 7- or 8-membered ring containing from one to three heteroatoms in the ring. Preferably, R⁷ and R⁸ are each independently a (C₁-C₈)alkyl group, or are combined to form a 5-, 6-, or 7-membered ring. R⁹ and R¹⁰ are independently selected from hydrogen, alkyl, aryl and arylalkyl. In preferred embodiments, R⁹ and R¹⁰ are independently selected from hydrogen, (C₁-C₈)alkyl, phenyl and phenyl(C₁-C₄)alkyl. R¹¹ is alkyl, aryl or arylalkyl, preferably, (C₁-C₈)alkyl, phenyl and phenyl(C₁-C₄)alkyl. R¹² is alkyl, preferably (C₁-C₄)alkyl, more preferably (C₁-C₃)alkyl, and even more preferably methyl.

In addition to the above descriptions of R¹ to R¹², the present formula above is meant to represent a number of compounds in which a second ring is fused to the pyrimidine ring, including structures in which one of the pyrimidine ring nitrogen atoms is at the ring junction. For the discussion below and the claims, the nitrogens are individually referred to as follows: N¹ is the nitrogen atom at the 1-position of the ring (which is between the carbon atom bearing —R² and the carbon atom bearing —Y—R⁵). N³ is the nitrogen atom at the 3-position of the pyrimidine ring (which is the nitrogen atom between the carbon bearing —Y—R⁵ and the carbon atom bearing —X). Examples of fused rings are those in which R¹ is joined to R², R¹ is joined to R³, R³ is joined to N³, R⁵ is joined to N³, R⁵ is joined to N¹ or R² is joined to N¹ to form a fused 5-, 6-, or 7-membered ring. The ring formed by these combinations will contain 1-3 heteroatoms (e.g., O, N or S) and can be either aromatic or nonaromatic. Preferably the additional ring formed is a 5- or 6-membered ring.

When R¹ and R² are combined to form a ring, the combination can be replaced with a substituent of the formula —T—C(O)—(CH₂)ₛ—U—, wherein T and U are independently selected from —NH—, —O—, —CH₂— or a single bond, and the subscript s is an integer of from 0 to 2. Alternatively, the R¹ and R² radicals can be replaced with a substituent of the formula —A—(CH₂)ₚ—B—, wherein A and B are independently selected from —CH₂—, —O—, —NH—, —S—, —S(O)—, —S(O)₂—, —S(O)₂NR′— or a single bond, and p is an integer of from 1 to 3. One or more of the single bonds of the new ring so formed may optionally be replaced with a double bond. Alternatively, the R¹ and R² radicals can be replaced with a substituent of the formula —(CH₂)_{q}—Z—(CH₂)ᵣ—, where q and r are independently integers of from 1 to 3, and Z is —O—, —NR′—, —S—, —S(O)—, —S(O)₂—, or —S(O)₂NR′—. The substituent R′ in —NR′— and —S(O)₂NR′— is selected from hydrogen or (C₁-C₆)alkyl.

The subscript m, in the groups above, is an integer of from 1 to 2, preferably 2. The subscript n, in the groups above, is an integer of from 1 to 3, preferably 2.

The compounds provided in the above formula are meant to include all pharmaceutically acceptable salts and prodrugs thereof.A number of substituent combinations on the pyrimidine ring are particularly preferred. In the following preferred embodiments, the substitutents X, Y and R¹ to R¹² are generally defined as above.

One group of preferred embodiments has the formula (IIa):

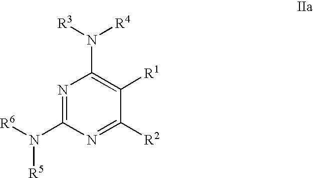

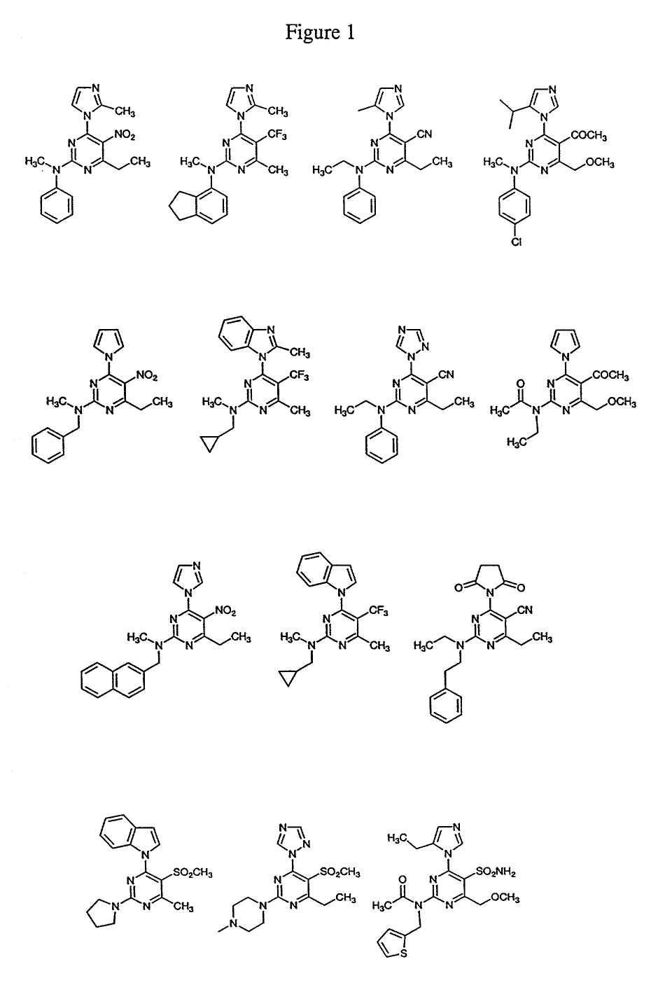

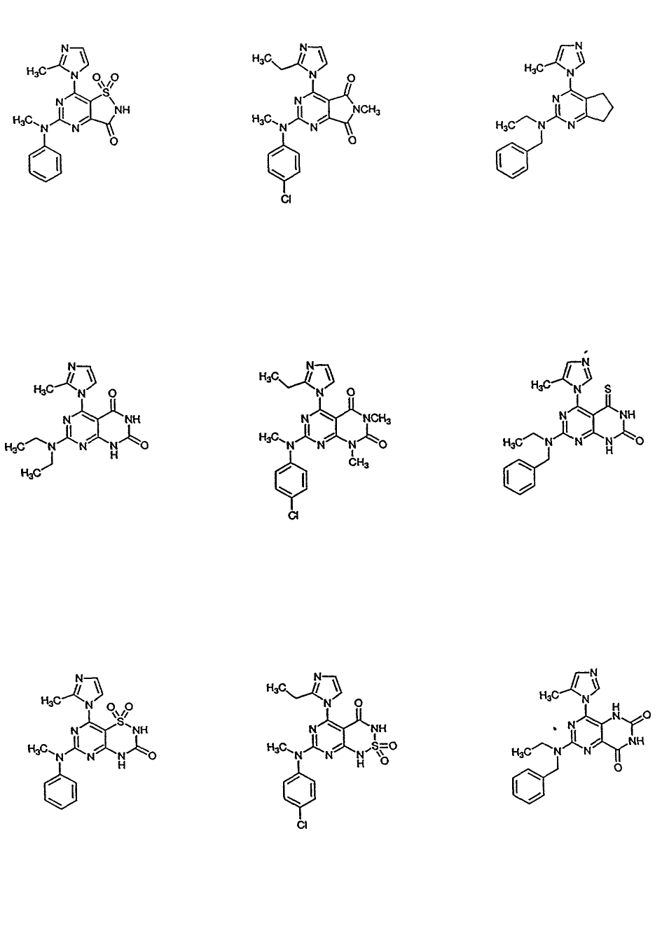

In compounds of general formula IIa, R¹ is preferably —NO₂, —CF₃, —C(O)NR⁷R⁸, —CO₂R¹⁰, —S(O)₂NR⁷R⁸, —S(O)₂R⁹, —C(O)R⁹, —SO₂NH₂, or —CN and R² preferably an alkyl group having from 1 to 8 carbon atoms. In the most preferred embodiments, the R³ and R⁴ groups are combined to form a 5-membered ring which is optionally fused to an aryl group. Examples of suitable 5-membered ring groups (and those which are optionally fused to an aryl group) include pyrrolidine, pyrrole, imidazole, pyrazole, benzimidazole, imidazoline, 1,2,4-triazole, 1,2,3-triazole, imidazolidin-2-one, and the like. More preferably, the R³ and R⁴ groups are combined to form an imidazole ring which is substituted or, optionally, is fused to an aryl group. Preferred substituted (and fused) imidazole rings include, for example, 2-methylimidazole, 2-ethylimidazole, 2-isopropylimidazole, 2-aminoimidazole, 5-methylimidazole, 5-ethylimidazole, 5-isopropylimidazole, 2,5-dimethylimidazole, benzimidazole, and 2-methylbenzimidazole. The R⁵ and R⁶ groups are independently selected from hydrogen, alkyl, aryl and arylalkyl, or can be combined to form a ring which is optionally fused to an aryl group. FIG. 1 provides exemplary structures of compounds within this preferred group of embodiments.

Another group of preferred embodiments are represented by the formula (IIb):

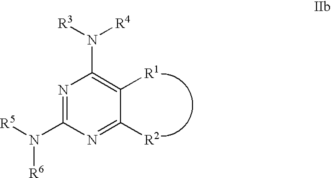

In this formula, the fused ring containing R¹ and R² is typically a heterocyclic ring in which the —R¹—R²— group is selected from, for example, —S(O)₂NR′C(O)—, —S(O)₂NR′C(O)NR″—, —NR′S(O)₂NR″C(O)—, —C(O)NR′C(O)—, —NR′C(O)NR″C(O)—, —NR′C(S)NR″C(O)—, —NR′C(S)NR″C(S)—, in which R′ and R″ are independently hydrogen or (C₁-C₈)alkyl. The R³ and R⁴ groups are preferably combined to form a 5-membered ring which is optionally fused to an aryl group. More preferably, the R³ and R⁴ groups are combined to form an imidazole ring which is optionally fused to an aryl group. The R⁵ and R⁶ groups are independently selected from hydrogen, alkyl, aryl and arylalkyl, or can be combined to form a ring which is optionally fused to an aryl group. FIG. 2 provides exemplary structures of compounds within this preferred group of embodiments.

Yet another group of preferred embodiments is represented by the formula (IIc):

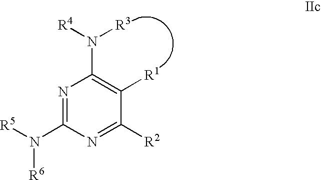

In this formula, the divalent radical —R¹—R³— is typically an alkylene group, —C(O)NR′C(O)—, —C(O)NR′S(O)₂— or —S(O)₂NR′C(O)—, in which R′ is a hydrogen or lower alkyl group. Preferably, R² and R⁴ will each independently be an alkyl group, more preferably a lower alkyl group. The R⁵ and R⁶ groups are independently selected from hydrogen, alkyl, aryl and arylalkyl, or can be combined to form a ring which is optionally fused to an aryl group. FIG. 3 provides exemplary structures of compounds within this preferred group of embodiments.

Still another group of preferred embodiments are represented by the formula (IId):

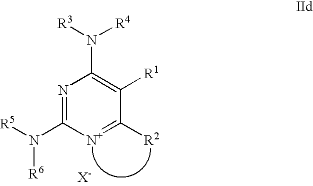

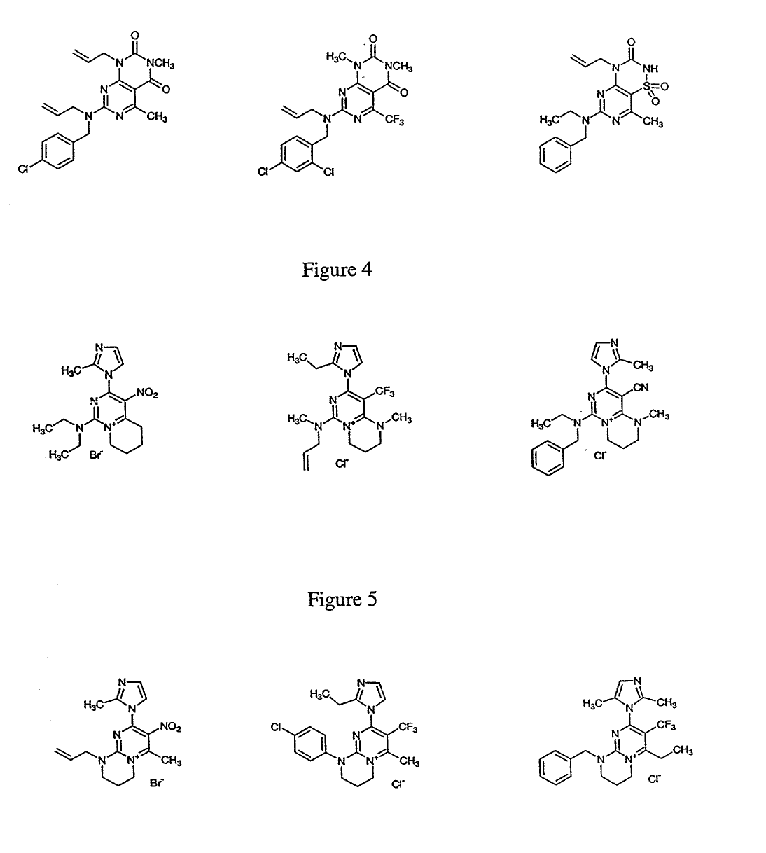

In this formula, the fused ring portion defined by —R²— is typically a (C₃-C₅)alkylene group, alkyleneamine group (e.g., —NHCH₂CH₂CH₂—, —NHCH₂CH₂—), or a —NR′C(O)CH₂— group, in which R′ is hydrogen or a lower alkyl group. R¹ is typically —NO₂, —S(O)₂NR⁷R⁸, —S(O)₂R⁹, —CN, —CF₃, —C(O)R⁹, —CO₂R¹⁰ or —C(O)NR⁷R⁸. More preferably, R¹ is —NO₂, —CN, —CF₃ or —CO₂R¹⁰, with —NO₂ being the most preferred. The R³ and R⁴ groups are preferably combined to form a 5-membered ring which is optionally fused to an aryl group. More preferably, the R³ and R⁴ groups are combined to form an imidazole ring which is optionally fused to an aryl group. The R⁵ and R⁶ groups are independently selected from hydrogen, alkyl, aryl and arylalkyl, or can be combined to form a ring which is optionally fused to an aryl group. The symbol X⁻ represents a suitable counterion for the quaternary nitrogen. Preferred counterions are those which form pharmaceutically acceptable salts. FIG. 4 provides exemplary structures of compounds within this preferred group of embodiments.

Another group of preferred embodiments are represented by the formula(IIe):

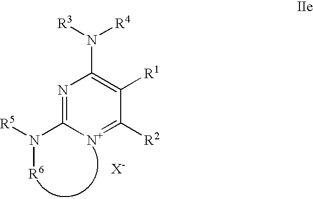

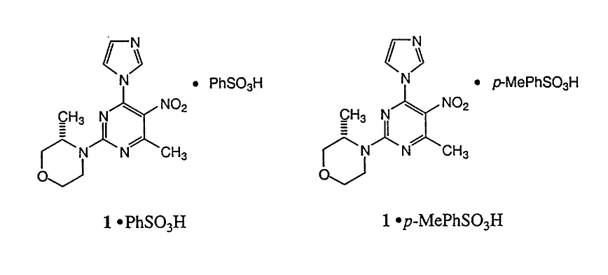

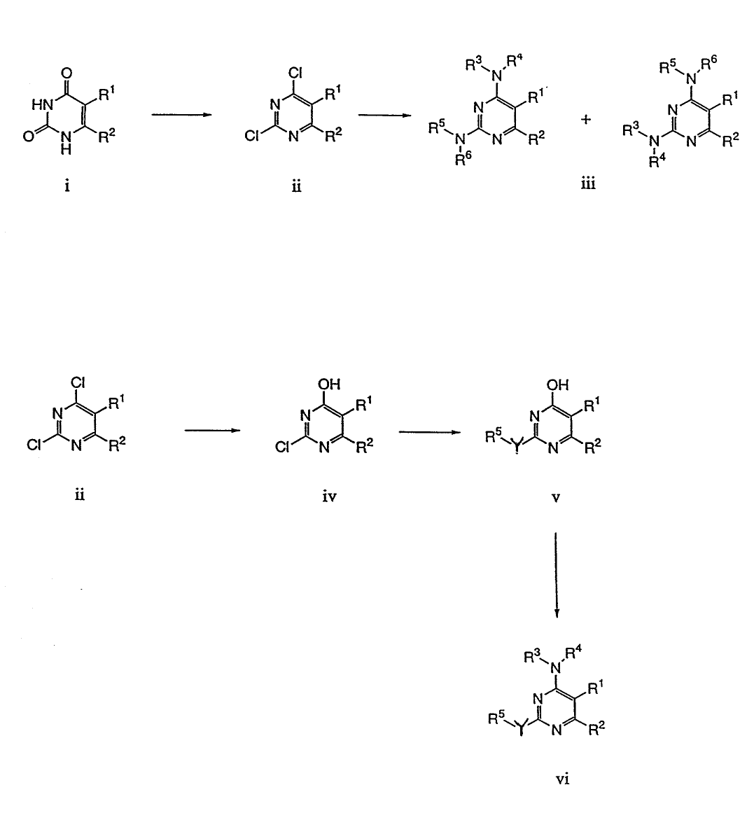

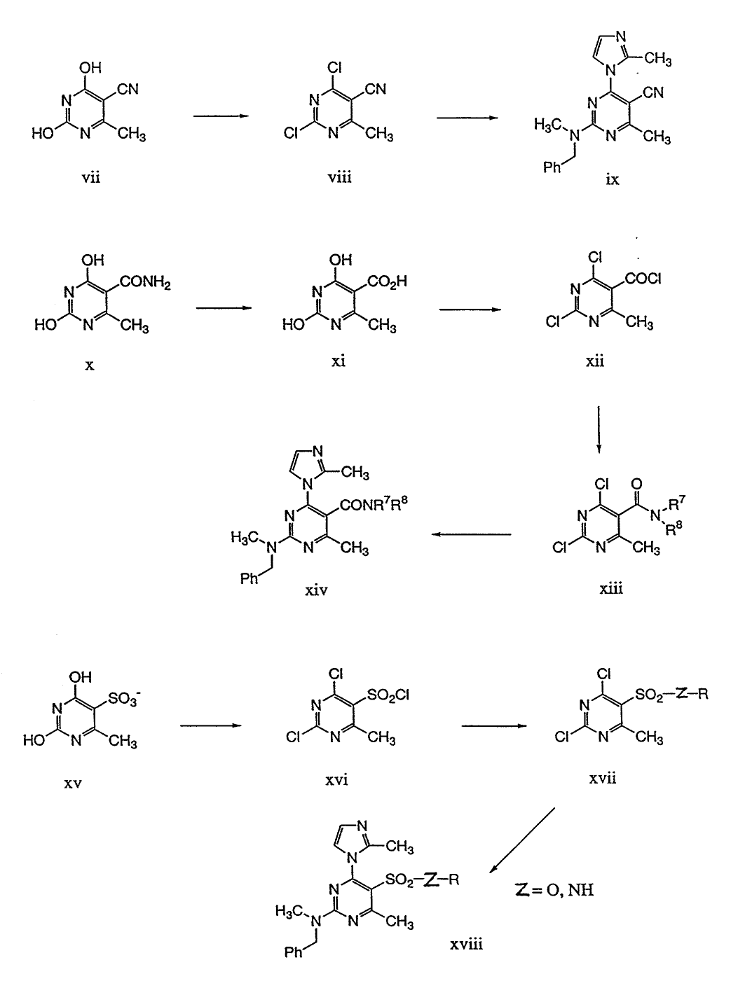

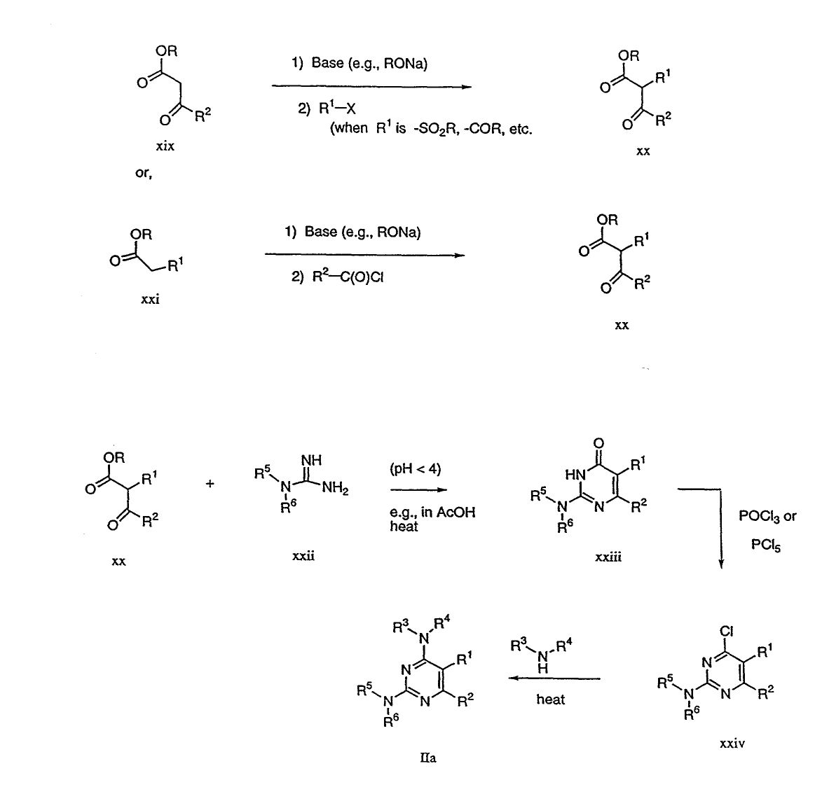

In this formula, R¹ is preferably —NO₂, —S(O)₂NR⁷R⁸, —S(O)₂R⁹, —CN, —CF₃, —C(O)R⁹, —CO₂R¹⁰ or —C(O)NR⁷R⁸. More preferably, R¹ is —NO₂, —CN, —CF³ or —CO₂R¹⁰, with —NO₂ being the most preferred. R² is preferably an alkyl group having from 1 to 8 carbon atoms. The R³ and R⁴ groups are preferably combined to form a 5-membered ring which is optionally fused to an aryl group. More preferably, the R³ and R⁴ groups are combined to form an imidazole ring which is optionally fused to an aryl group. R⁵ is preferably hydrogen, (C₁-C₈)alkyl, phenyl, or phenylalkyl. The fused ring portion defined by —R⁶— is typically a (C₃-C₅)alkylene group or a substituted alkylene group (e.g., —C(O)CH₂CH₂CH₂—, —C(O)CH₂CH₂—), or a —NR′C(O)CH₂— group, in which R′ is hydrogen or a lower alkyl group. The symbol X⁻ represents a suitable counterion for the quaternary nitrogen. Preferred counterions are those which form pharmaceutically acceptable salts. FIG. 5 provides the structures of exemplary compounds of formula IIe.

Yet another group of preferred embodiments is represented by the formula (IIf):

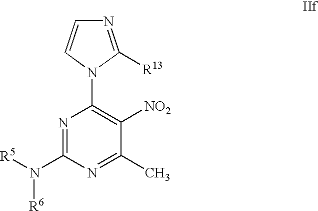

In this formula R¹³ is preferably hydrogen, methyl or ethyl. Preferably, R⁵ and R⁶ are combined with the nitrogen atom to which R⁵ and R⁶ are attached to form a ring selected from the group consisting of substituted or unsubstituted 1-piperidinyl, substituted or unsubstituted 4-morpholinyl and substituted or unsubstituted 1-pyrrolidinyl. FIG. 6 provides exemplary structures of compounds within this preferred group of embodiments.

### Compositions

In another aspect, the invention provides pharmaceutical compositions which are suitable for pharmaceutical or diagnostic use. The compositions comprise compounds of formula I provided above, in combination with a diagnostically or pharmaceutically acceptable excipient. The subject compositions are useful for treating diseases associated with CMV infection, such as atherosclerosis and restenosis, organ transplant rejection and pathologies associated with organ transplantation. The compositions are also useful for treating diseases produced by CMV infection, such as retinitis, mononucleosis, pneumonitis and hepatitis. Suitable pharmaceutically acceptable excipients include sterile saline or other medium, water, gelatin, an oil, etc. The compositions and/or compounds may be prepared in combination with any convenient carrier, diluent, etc. Useful carriers include solid, semi-solid or liquid media including water and non-toxic organic solvents.

Solid form preparations include powders, tablets, pills, capsules, cachets, lozenges, troches, hard candies, powders, sprays, creams, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg according to the particular application and the potency of the active component. The composition can, if desired, also contain other compatible therapeutic agents.

The compositions may be advantageously combined and/or used in combination with agents useful in the treatment and/or prevention of atherosclerosis (e.g., cholestyramine used to reduce cholesterol) and/or restenosis, organ transplant rejection (e.g., sirolimus) and pathologies associated with organ transplantation, described herein. The compositions may also be advantageously combined and/or used in combination with agents useful in the treatment and/or prevention of pathologies associated with organ transplantation, such as lymphoproliferative disorders and thrombosis. In many instances, administration of the subject compounds or compositions in conjunction with these alternative agents enhances the efficacy of such agents. Accordingly, in some instances, the present compounds, when combined or administered in combination with anti-atherosclerotic and/or anti-restenotic agents and/or immunosuppressive agents, can be used in dosages which are less than the expected amounts when used alone, or less than the calculated amounts for combination therapy.

Suitable agents for combination therapy include those that are currently commercially available and those that are in development or will be developed. Exemplary agents useful in the treatment of atherosclerosis and/or restenosis include antithrombotic agents, lipid lowering agents, calcium channel blockers, angiotensin converting enzyme (ACE) inhibitors, smooth muscle growth inhibitors and antioxidant agents.

The compositions may also be advantageously combined and/or used in combination with antiviral agents useful in the treatment and/or prevention of the viral infections described herein. The compositions may also be advantageously combined and/or used in combination with agents useful in the treatment and/or prevention of conditions often associated with the viral infections described herein, such as anti-HIV agents (described below), immunostimulatory agents (e.g., vaccines) or immunosuppressive agents (e.g., cyclosporin, FK-506 (tacrolimus) and rapamycin (sirolimus)). In many instances, administration of the subject compounds or compositions in conjunction with these alternative agents enhances the efficacy of such agents. Accordingly, in some instances, the present compounds, when combined or administered in combination with antiviral or immunosuppressive agents, can be used in dosages which are less than the expected amounts when used alone, or less than the calculated amounts for combination therapy. Such combination therapy often is advantageous because a reduction in dose of one or more agents frequently results in a decrease in the adverse effects associated with the agent(s).

While antiviral agents may be particularly suitable for the treatment or prevention of a particular viral disorder(s), practitioners skilled in the art understand that such agents frequently are useful in treating a range of viral-related disorders. Exemplary agents useful in the treatment of CMV include acyclovir, cidofovir, ganciclovir, valganciclovir, immunoglobulin (CMV-specific and unselected) and foscarnet. Other promising anti-CMV agents include (a) the nucleoside/nucleotide analogs valaciclovir, adefovir, dipivoxil and lobucavir; (b) the antisense agents fomivirsen, GEM 132 (Hybridon), ISIS 13312 (ISIS) and (c) other therapies like benzimidavir and sevirumab.

Exemplary anti-HIV agents include (a) nucleoside analog reverse transcriptase inhibitors such as zidovudine (AZT), didanosine (ddI), zalcitabine (ddC, dideoxycytidine), stavudine (d4T), lamivudine (3TC), abacavir (1592U89), emtricitabine (FTC, Triangle Pharmaceuticals), BCH-10652 (BioChem Pharma) and the related nucleotide analogs (e.g., PMPA (Gilead Sciences)); (b) non-nucleoside reverse transcriptase inhibitors such as nevirapine (NVP), delavirdine (DLV), efavirenz (DMP-266), emivirine (MKC-442), AG1549 (Agouron Pharmaceuticals; PNU142721 (Pharmacia), calanolide-A (Sarawak MediChem Pharmaceuticals); (c) protease inhibitors such as saquinavir (SQV), ritonavir (RTV), indinavir (IDV), nelfinavir (NFV) saquinavir (SQV), amprenavir (APV), 232,632 (Bristol-Myers Squibb), tipranavir, DMP-450 (Triangle Pharmaceuticals), and lopinavir and (d) immune stimulators such as interleukin 2 (Chiron), Reticulose® (Advance Viral Research Corporation), Multikine® (Cel-Sci Corporation), and HIV-1 immunogen (Immune Response Corporation). Other anti-HIV agents that may be used in combination with the compounds and compositions of the present invention include HIV integrase inhibitors (e.g., AR-177 (Aronex Pharmaceuticals)), fusion inhibitors (e.g., T-20 (Roche)) and antisense drugs (e.g., HGTV43 (Enzo Therapeutics)).

### Methods of Use

In yet another aspect, the present invention provides novel methods for the use of the foregoing compounds and compositions. In particular, the invention provides novel methods for treating or preventing diseases associated with CMV infection, preferably cardiovascular disease, such as atherosclerosis and restenosis, and organ transplant rejection, including heart transplant rejection, kidney transplant rejection, lung transplant rejection, liver transplant rejection and bone marrow transplant rejection, as known in the art. The methods typically involve administering to a patient an effective formulation of one or more of the subject compositions.

In still another aspect, the invention provides methods of using the subject compounds and compositions to treat disease or provide medicinal prophylaxis to individuals who possess a compromised immune system or are expected to suffer immunosuppressed conditions, such as patients prior to undergoing immunosuppressive therapy in connection with organ transplantation or anticancer chemotherapy. These methods generally involve administering to the host an effective amount of the subject compounds or pharmaceutically acceptable compositions.

The compositions and compounds of the invention and the pharmaceutically acceptable salts thereof can be administered in any effective way such as via oral, parenteral or topical routes. Generally, the compounds are administered in dosages ranging from about 2 mg up to about 2,000 mg per day, although variations will necessarily occur depending on the disease target, the patient, and the route of administration. Preferred dosages are administered orally in the range of about 0.05 mg/kg to about 20 mg/kg, more preferably in the range of about 0.5 mg/kg to about 10 mg/kg, most preferably in the range of about 1 mg/kg to about 5 mg per kg of body weight per day.

It is believed that the compounds of the invention will block CMV replication by specifically modulating or inhibiting the activity of CMV DNA primase. CMV DNA primase regulates initiation of CMV DNA replication. Therefore, inhibition of CMV DNA primase will inhibit CMV DNA replication and render the virus unable to reproduce.

While a precise understanding of the mechanism by which compounds inhibit viral primase activity is not required in order to practice the present invention, it is believed that the compounds interact with a cysteine residue of the CMV UL70 protein, which mediates CMV DNA primase activity. In particular, it is believed that the compounds covalently modify cysteine residue 570 (Cys₅₇₀) of a deleted amino-terminal sequence of UL70.

Full length human CMV UL70 (SwissProt Accession No. P17149) has been described; see, e.g., “Chee et al. (1990) Curr. Top. Microbiol. Immunol. 15:125-169, and has the sequence shown in SEQ ID NO:1. Cys₅₇₀ of the deleted amino-terminal sequence described herein corresponds to Cys₆₈₆ of the full length sequence.

The compounds possess an electrophilic moiety that is capable of reacting with a thiol group. Specifically, the compounds of the invention bind covalently to Cys₅₇₀ of the CMV UL70 protein, and this binding is specific. Compounds contemplated by the invention include, but are not limited to, the exemplary compounds provided herein. The skilled practitioner can propose additional compounds possessing an electrophilic moiety that will react with Cys₅₇₀ of UL70 in a similar manner.

### Preparation of the Compounds

The compounds of the present invention can be prepared using general synthesis schemes, such as those outlined in FIGS. 7-16. One of skill in the art will understand that the syntheses provided below can be modified to use different starting materials and alternate reagents to accomplish the desired transformations. Accordingly, the description below, the Figures and the reagents are all expressed as non-limiting embodiments.

Briefly, the compounds of formula I, in which Y is —N(R⁶)— can be prepared from a variety of known pyrimidinediones. As shown in FIG. 7, the pyrimidine dione (i) can be converted to the corresponding dichloride (ii) by treatment with reagents such as, for example, POCl₃. Treatment of ii with the desired amines (including heterocyclic amines) provides the target compounds, typically as a mixture of isomers (iii). Separation of the isomers can be accomplished by traditional methods such as column chromatography or HPLC. Alternatively, ii can be hydrolyzed to a mono chloro compound (using, for example, sodium acetate, acetic acid, water and ethanol) to provide (iv) which upon treatment with a suitable amine, alkoxide or thiolate ion provides (v). Conversion of the 4-hydroxy group to a 4-chloro substituent and displacement with a suitably nucleophilic amine provides the targets (vi).

A number of pyrimidinediones are commercially available and can be used as starting materials for the above transformations, including, for example, 5-cyano-6-methyl-2,4-pyrimidinedione (vii), 6-methyl-2,4-pyrimidinedione-5-carboxamide (x), 6-methyl-2,4-pyrimidinedione-5-sulfonic acid (xv) and 6-methyl-5-nitro-2,4-pyrimidinedione. Each of these compounds can be converted to target compounds of formula (IIa) as illustrated in FIG. 8. For example, 5-cyano-6-methyl-2,4-pyrimidinedione (vii) can be converted to a dichloride (viii) using reagents such as POCl₃, then further converted to target compounds (e.g., ix) upon treatment with amines R³—NH—R⁴ (e.g., 2-methylimidazole) and R⁵—NH—R⁶ (e.g., N-methylbenzylamine).

The carboxamide group of 6-methyl-2,4-pyrimidinedione-5-carboxamide (x) can be hydrolyzed to a carboxylic acid (xi) with aqueous base and then converted to an acid chloride (xii) with POCl₃ (forming a trichloride). Stepwise addition of amines or other suitable nucleophiles provides the target compounds (e.g., xiv). Similarly, a trichloride (xvi) is formed by treating 6-methyl-2,4-pyrimidinedione-5-sulfonic acid (xv) with chlorinating agents such as POCl₃. Again, the stepwise addition of amines or other suitable nucleophiles produces the desired target species (xviii).

Yet another method for the preparation of compounds of formula IIa is shown in FIG. 9. Treatment of either a β-ketoester (xix) or an a-methylene ester (xxi) with base (e.g., sodium alkoxide) and an electrophile (e.g., an alkylating agent, acylating agent, sulfonylating agent, and the like) provides a suitably derivatized β-ketoester (xx) which can be converted to a pyrimidinone (xxiii) upon treatment with a substituted guanidine (xxii), typically in acid (acetic acid) with heating. The substituents in the 5- and 6-positions (R¹ and R², respectively) are determined by the groups present on the derivatized β-ketoester. Chlorination of the pyrimidinone to produce (xxiv) and subsequent treatment with a nucleophilic nitrogen heterocycle (e.g., imidazole, 2-alkylimidazole, pyrrolidine, piperidine and the like) as well as other amines provides the target compounds of formula IIa. Substituted guanidines used in this method of preparation can either be obtained from commercial sources or can be prepared by the treatment of a secondary amine with cyanamide. Additional literature methods for the preparation of substituted guanidines are known to those of skill in the art.

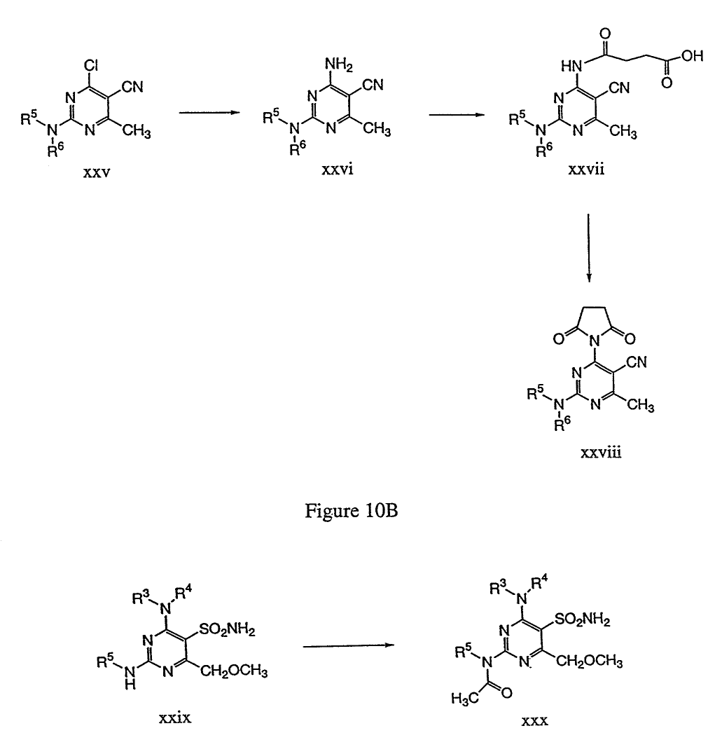

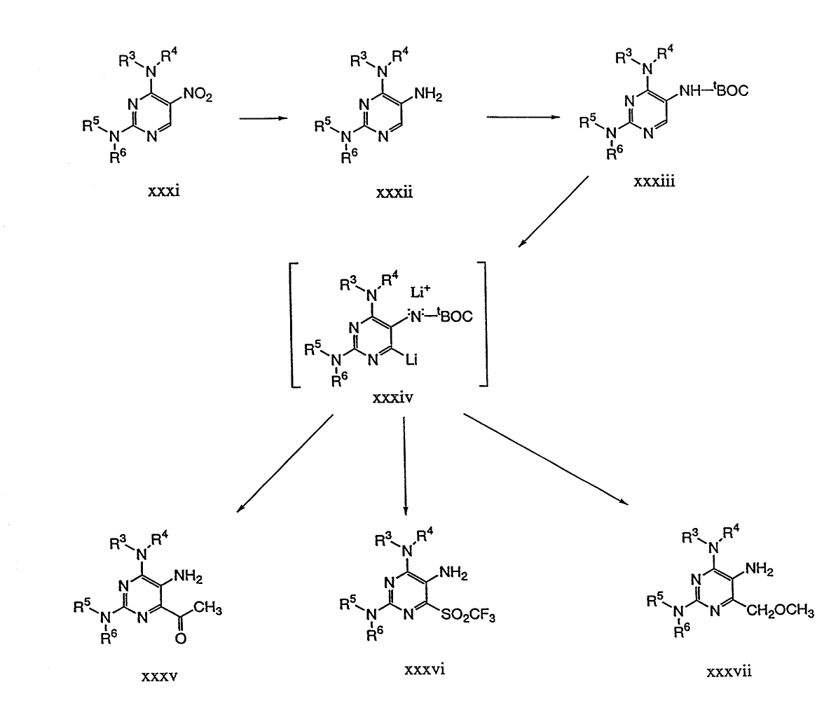

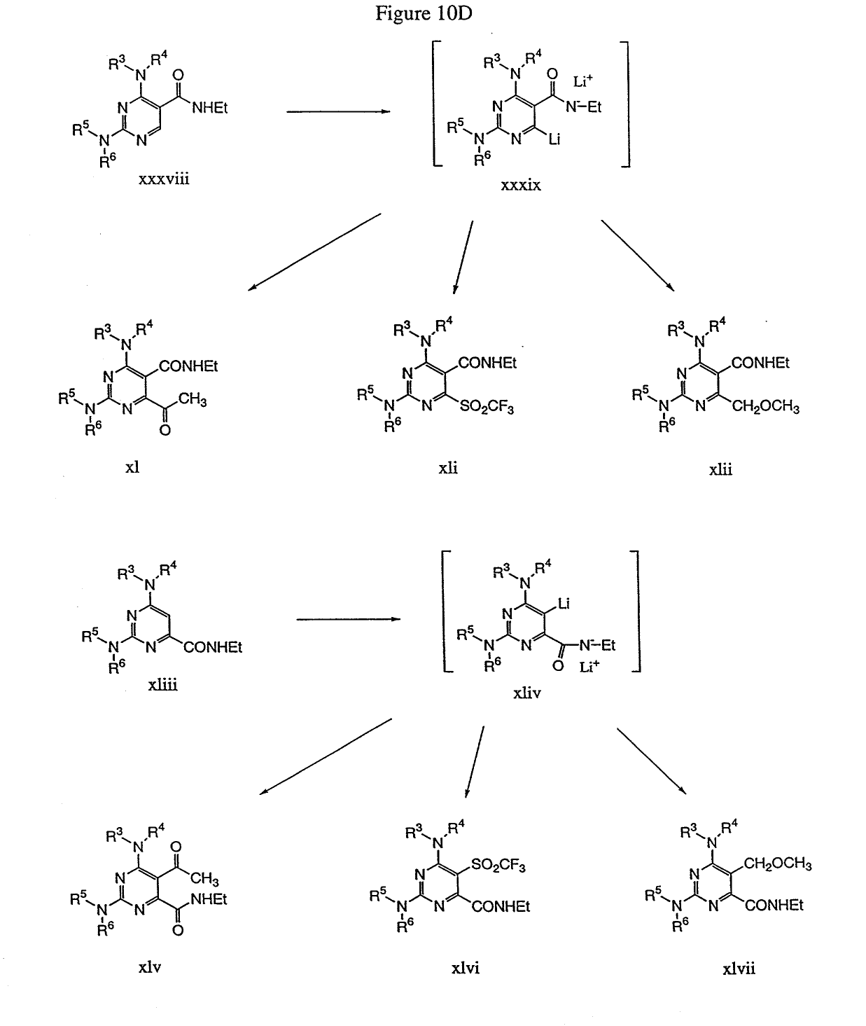

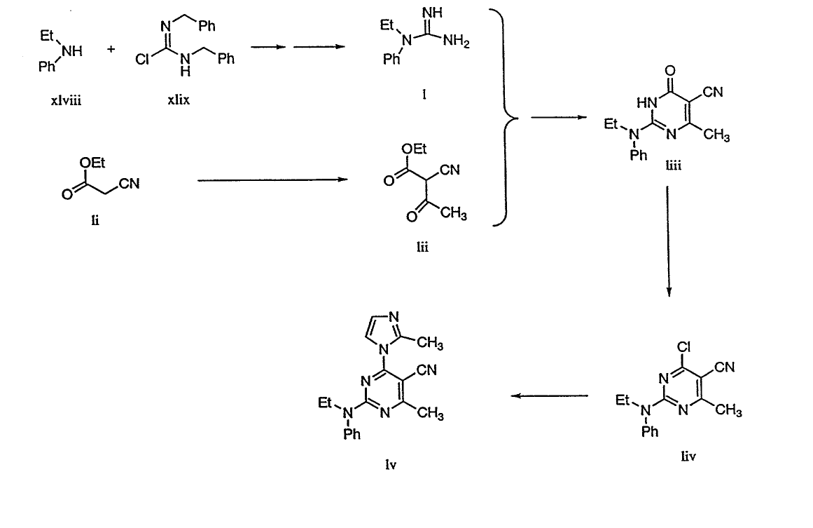

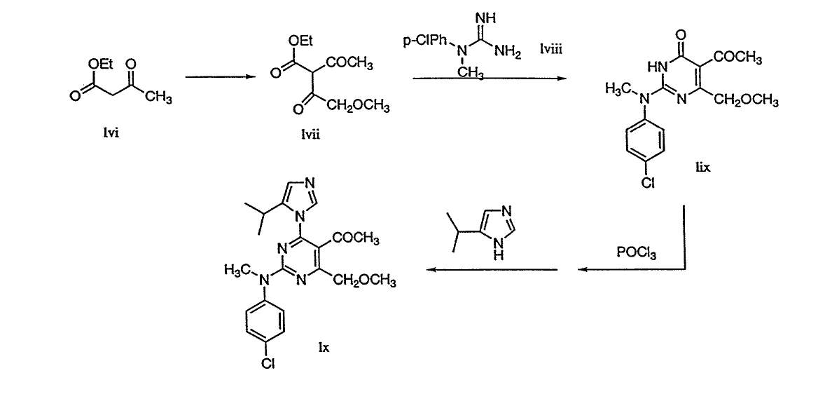

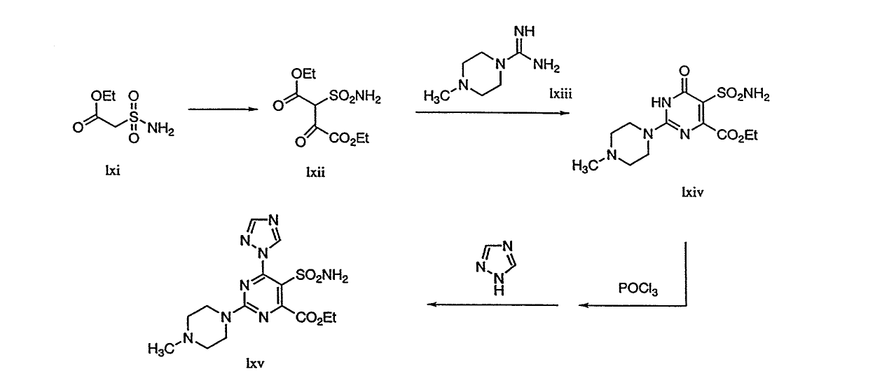

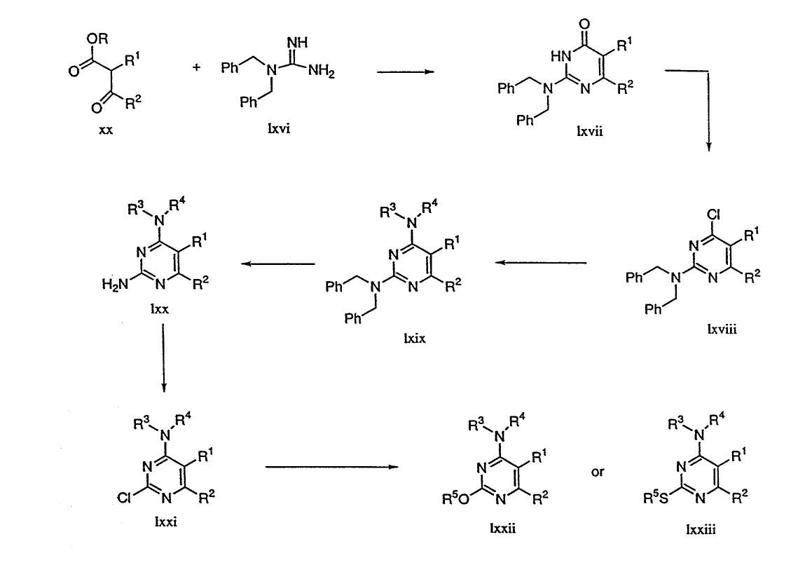

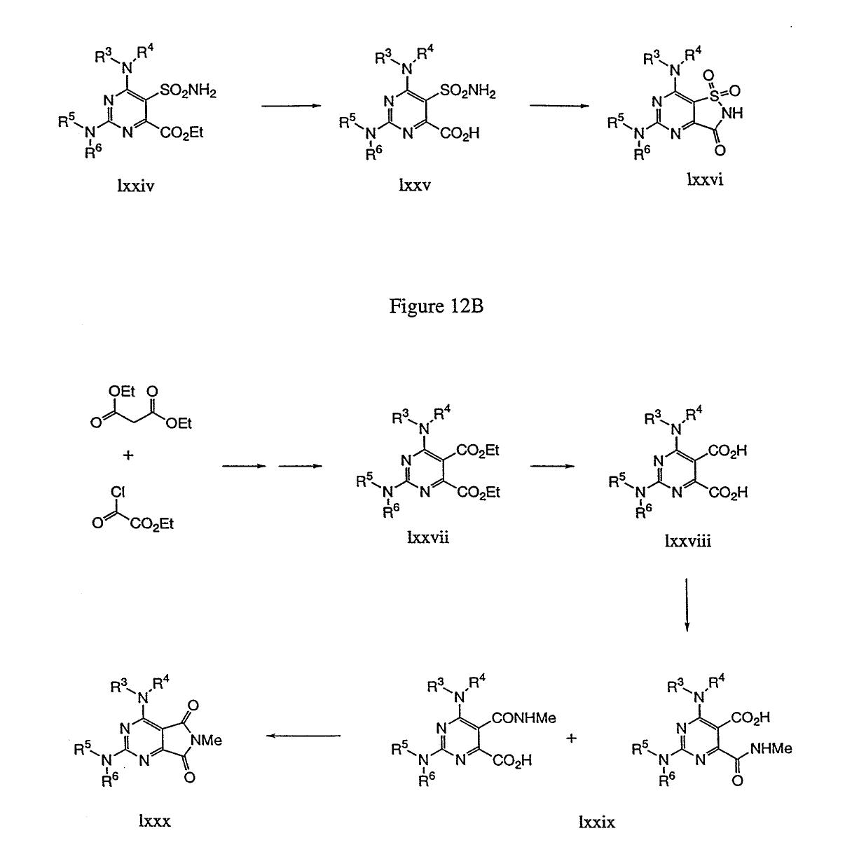

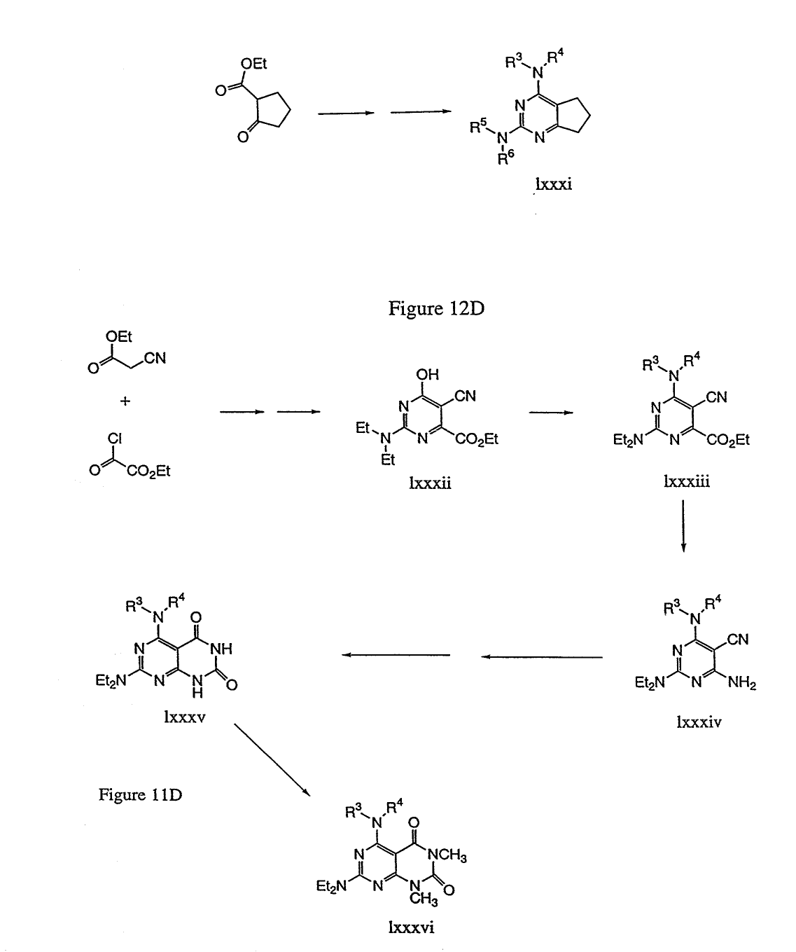

A number of transformations can be carried out to attach groups to an unsubstituted position on the pyrimidine ring, or to modify existing groups (see FIG. 10). For example, a 4-chloro substituent (present, for example, in xxv) can be displaced with ammonia to produce a 4-aminopyrimidine (e.g., xxvi). Treatment of the primary amine with succinic anhydride provides (xxvii) which upon treatment with acetic anhydride produces the succinimide compound xxviii (FIG. 10A). Exocyclic amino groups can also be acylated using standard acylating agents as shown in FIG. 10B. Metallation reactions can be carried out on pyrimidines which are unsubstituted in the 6-position (FIG. 10C). For example, a 5-nitropyrimidine derivative (xxxi) can be catalytically (H₂) or chemically (e.g., Fe/HCl) reduced to a 5-aminopyrimidine derivative (xxxii) which is then protected as a t-butyl carbamate (xxxiii). Treatment of the protected 5-aminopyrimidine derivative with a metallating agent such as sec-butyllithium provides a metallated intermediate (xxxiv) which can be acylated (xxxv), sulfonylated (xxxvi) or alkylated (xxxvii), as shown. Similarly (see FIG. 11D), the pyrimidine derivative (xxxviii) can be metallated to produce intermediate (xxxix), then acylated (xl), sulfonylated (xli) or alkylated (xlii). Introduction of functional groups at the 5-position can be accomplished using similar metallation chemistry on, for example, the pyrimidine derivative (xliii), to produce intermediate (xliv) which can be acylated (xlv), sulfonylated (xlvi) and alkylated (xlvii).

FIG. 11A-11D provides synthesis schemes for several compounds which follow the general methods shown in FIGS. 7-9. For example, FIG. 11A illustrates the preparation of a substituted guanidine (l) from a secondary amine (xlviii) and a chloroimidate (xlix) and the conversion of ethyl cyanoacetate (li) to the ketoester (lii). Condensation of l and lii produces the pyrimidinone (liii) which can be chlorinated to provide liv and then treated with an amine nucleophile (e.g., 2-methylimidazole) to provide the target lv. FIG. 10B illustrates a similar route in which ethyl acetoactate (lvi) is acylated to provide the tricarbonyl compound (lvii). Condensation of lvii with the substituted guanidine (lviii) provides the pyrimidinone (lix) which is converted to the target (lx) using standard protocols. FIG. 11C illustrates methodology in which a sulfonamide group is present in the starting material (lxi) and the substituted guanidine (lxiii) contains a nitrogen heterocycle. Accordingly, condensation of lxii and lxiii provides the pyrimidinone (lxiv) which is converted to the target (lxv) using POCl₃ (or other chlorinating agents) followed by reaction with an amine nucleophile (e.g., 1,2,4-triazole). Additionally, the general methodology allows the preparation of compounds having —O—Ar, —S—Ar, —O-alkyl and —S-alkyl groups at the 2-position of the pyrimidine ring (FIG. 10D). For example, treatment of the ketoester (xx) with the substituted guanidine (lxvi) provides the pyrimidinone (lxvii) which can be chlorinated and condensed with R³—NH—R⁴ to provide lxix. Removal of the protecting groups yields the 2-aminopyrimidine compound (lxx). Diazotization and subsequent chlorination can be carried out using standard procedures to provide lxxi. Displacement of the chloride with either an oxygen-containing nucleophile or a sulfur-containing nucleophile provides the target compounds lxxii or lxxiii, respectively.

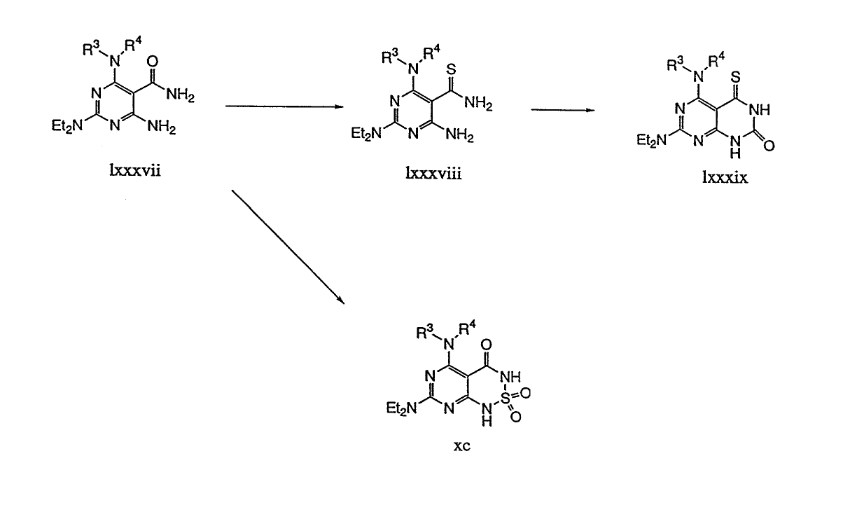

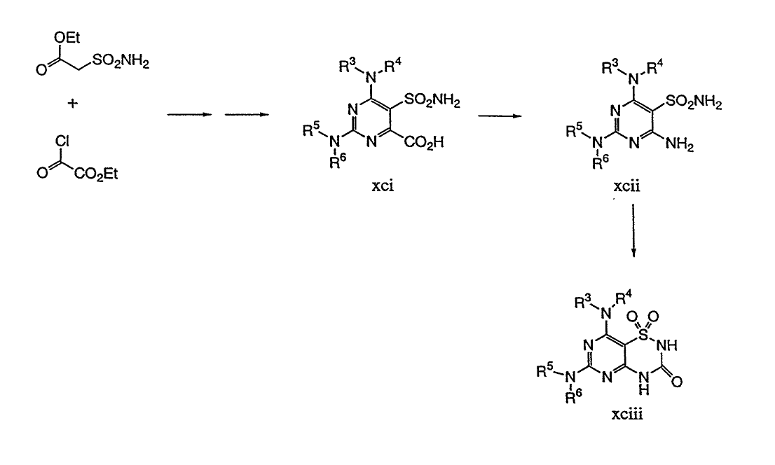

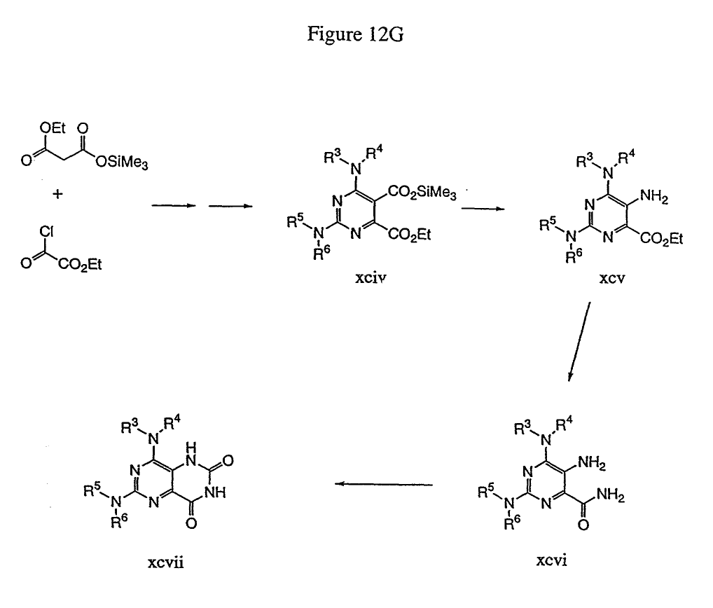

FIG. 12 illustrates the preparation of several compounds of formula IIb. In one group of embodiments, substituted pyrimidines having a sulfonamide at the 5-position and an ester group at the 6-position (lxxiv) can be saponified to provide lxxv, which is then cyclized with dehydrating agents (e.g., sulfuric acid or acetic anhydride) to the fused heterocycle shown as lxxvi (see FIG. 12A). In other embodiments, diesters (lxxvii) are saponified to the diacid (lxxviii) and converted to a mixture of amides (lxxix, by sequential treatment with acetic anhydride and methylamine), which can then be cyclized by treatment with a dehydrating agent (e.g., acetic anhydride) as indicated to provide a bicyclic system (lxxx, see FIG. 12B). Yet another fused bicyclic system (lxxxi) can be prepared beginning with ethyl 2-oxocyclopentanecarboxylate, using methods outlined above for the conversion of a β-ketoester to a substituted pyrimidine (see FIG. 12C). Still another group of embodiments can be prepared via manipulation of nitrile and ester substituents (see FIG. 12D). Briefly, ethyl cyanoacetate is first condensed with ethyl oxalyl chloride and the resultant product is treated with a substituted guanidine (exemplified herein with N,N-diethylguanidine) to provide the substituted pyrimidinone (lxxxii). Treatment of lxxxii with POCI₃ (or other chlorinating agent) followed by an appropriate amine (e.g., imidazole, 2-alkylimidazole, isopropylethylamine, pyrrolidine) provides the substituted pyrimidine (lxxxiii). Ester hydrolysis and Curtius rearrangement (using, for example, diphenylphosphoryl azide) provide the amino nitrile (lxxxiv). Conversion of the nitrile group to an amide by acid hydrolysis, and subsequent treatment with phosgene (or a phosgene equivalent such as diphosgene or dimethylcarbonate) provides the fused bicyclic system, lxxxv which can be further converted to lxxxvi on treatment with strong base (e.g., NaH) and an alkylating agent (e.g., MeI). Certain intermediates along these synthetic routes can be converted to other useful derivatives (FIG. 12E). For example, lxxxvii can be treated with Lawesson's reagent to provide the thioamide lxxxviii, which on treatment with phosgene (or a phosgene equivalent) provides the fused bicyclic system lxxxix. Alternatively, lxxxvii can be treated with sulfuryl chloride in the presence of a tertiary amine base to provide the fused bicyclic system xc. FIGS. 12F and 12G illustrate other methods of preparing compounds within the scope of formula IIb. In FIG. 12F, a substituted pyrimidine (xci) having a sulfonamide at the 5-position and a carboxylic acid at the 6-position is prepared using methods analogous to those described above. Curtius rearrangement of the carboxylic acid group in xci to an amino group provides xcii, which is then cyclized to xciii, using phosgene or a phosgene equivalent. FIG. 12G shows the preparation of a pyrimidine diester (xciv) and its conversion to the fused bicyclic system xcvii. Briefly, the silyl ester present in xciv is hydrolyzed to the acid which is subjected to a Curtius rearrangement to provide xcv. Conversion of the remaining ester group to an amide can be accomplished using standard procedures to provide xcvi. Cyclization of xcvi to xcvii can be carried out using phosgene or a phosgene equivalent.

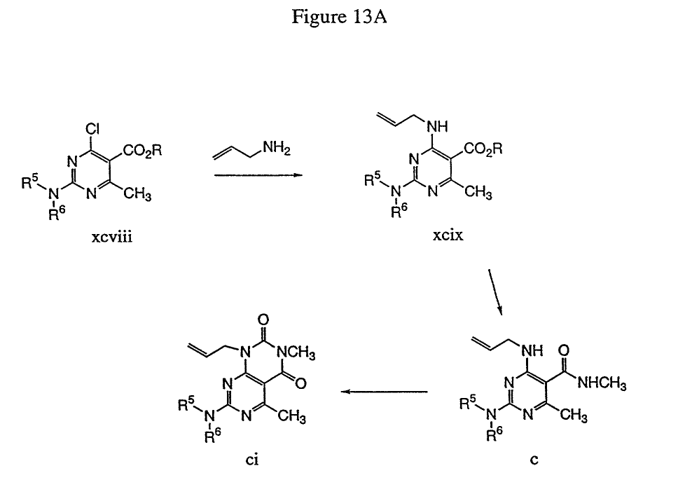

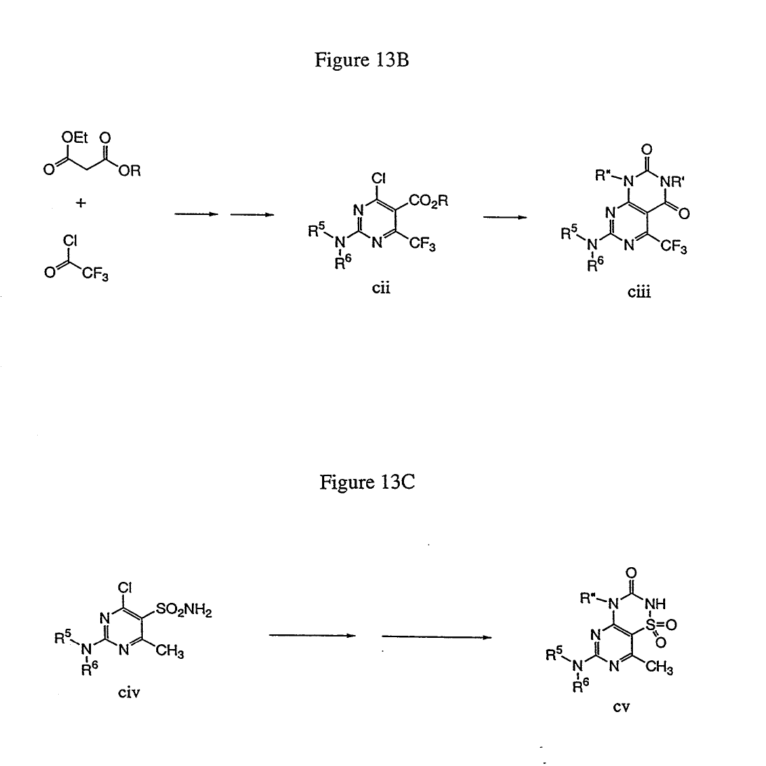

Compounds of formula IIc can be prepared by methods outlined in FIG. 13. In one group of embodiments (in FIG. 13A), a 4-chloropyrimidine derivative (xcviii, prepared by methods described above), is treated with an amine (e.g., allylamine) to provide xcix. The ester group is then converted to an N-methyl amide (c) upon treatment with methylamine in an alcohol solvent. Cyclization of c to ci occurs upon treatment with phosgene or an equivalent. Similarly, compounds having more electronegative groups in the 6-position can be prepared as shown in FIG. 13B. For example, the chloropyrimidine cii can be produced using methods outlined above and then converted to the bicyclic compound ciii, using procedures described for xcix. Still other fused systems of formula IIc can be prepared as shown in FIG. 13C. Here, a chloropyrimidine derivative (civ) is treated with a primary amine (e.g., allylamine) to provide an amino moiety at the 4-position of the pyrimidine ring. Cyclization of the amino moiety onto a sulfonamide (present at the 5-position) can be accomplished with phosgene or an equivalent to provide the target (cv).

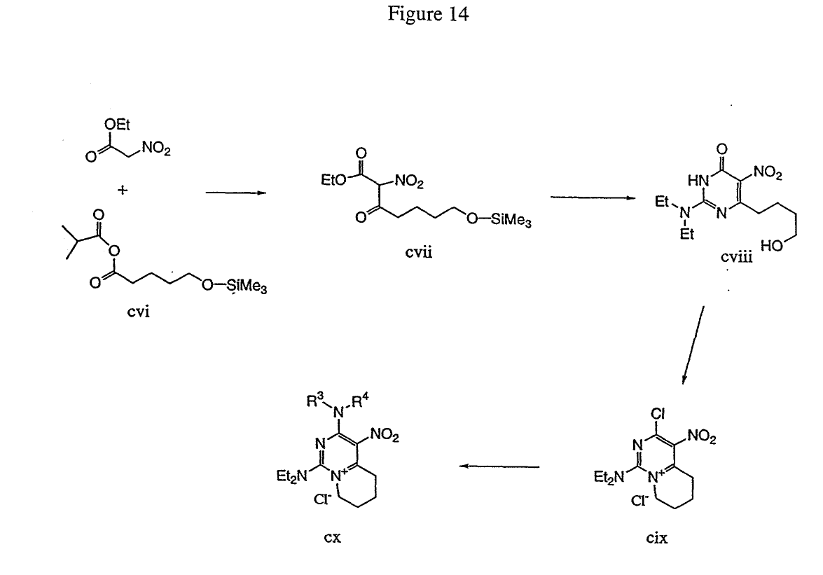

Preparation of compounds of formula IId can be accomplished, in one embodiment, as outlined in FIG. 14. Briefly, ethyl nitroacetate can be condensed with a mixed anhydride (cvi) to provide a nitroketoester (cvii) which can then be converted to a pyrimidine (cviii) upon treatment with a suitably substituted guanidine. Removal of the protecting group, followed by treatment with POCl₃ effects chlorination of the pyrimidine ring and cyclization to form a pyrimidinium salt (cix). Treatment of cix with an amine nucleophile produces the target compound (cx). Other compounds in this group can be prepared by starting with ethyl 3,3,3-trifluoropropionate or ethyl cyanoacetate and varying both the substituted guanidine and the amino nucleophile which are used.

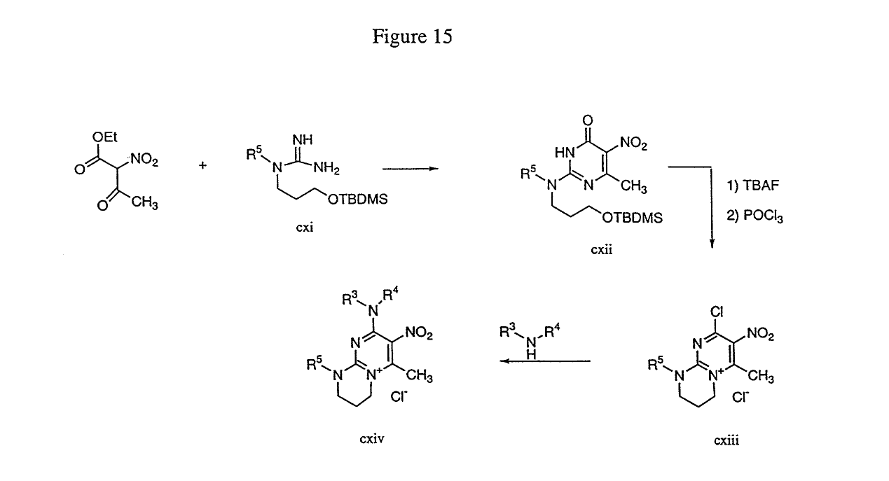

Preparation of certain compounds of formula IIe can be accomplished following procedures outlined in FIG. 15. According to the scheme depicted in FIG. 15, a suitably substituted guanidine (cxi, prepared from a protected hydroxypropylamine) is condensed with ethyl 2-nitroacetoacetate (or similarly ethyl 2-trifluoromethylacetoacetate) to provide the a pyrimidinone (cxii). Removal of the protecting group, chlorination and cyclization using procedures similar to those shown in FIG. 15, produces the salt (cxiii). Subsequent treatment of cxiii with a nucleophilic amine produces the target (cxiv).

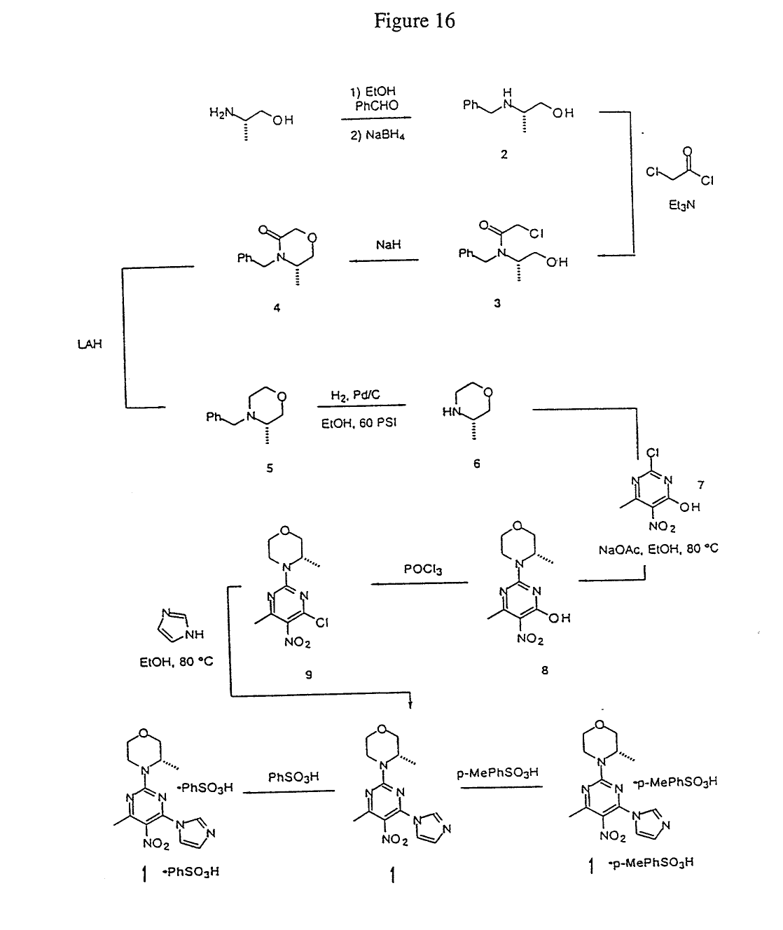

Preparation of compounds of formula IIf can be accomplished following procedures outlined in FIG. 16. Accordingly, (S)-2-aminopropanol can be treated with benzaldehyde in ethanol followed by sodium borohydride to form the N-benzyl alcohol 2. Acylation of the amine with chloroacetyl chloride provides 3, which can be cyclized to 4 upon treatment with sodium hydride. Reduction of the amide carbonyl present in 4 with lithium aluminum hydride (LAH) provides the substituted morpholine 5. Hydrogenolysis of the N-benzyl group can be accomplished with hydrogen using a palladium on carbon catalyst to provide (S)-3-methylmorpholine 6.

Compound 6 can be combined with 2-chloro-4-hydroxy-6-methyl-5-nitropyrimidine (7), to provide compound 8. The hydroxy group present in 8 can then be converted to a chlorine upon treatment with POCl₃ to provide compound 9, which upon treatment with imidazole in ethanol yields the parent compound 1. Conversion of 1 to the various salts can then be accomplished upon treatment with an equivalent of a suitable sulfonic acid (illustrated in FIG. 16 as benzenesulfonic acid (PhSO₃H) and toluenesulfonic acid (p-MePhSO₃H)).

The compounds used as initial starting materials in this invention may be purchased from commercial sources or alternatively are readily synthesized by standard procedures which are well know to those of ordinary skill in the art.

Some of the compounds of the present invention will exist as stereoisomers, and the invention includes all active stereoisomeric forms of these compounds. In the case of optically active isomers, such compounds may be obtained from corresponding optically active precursors using the procedures described above or by resolving racemic mixtures. The resolution may be carried out using various techniques such as chromatography with a chiral solid support or a chiral solvent, repeated recrystallization of derived asymmetric salts, or derivatization, which techniques are well known to those of ordinary skill in the art.

The compounds of the invention may be labeled in a variety of ways. For example, the compounds may contain radioactive isotopes such as, for example, ³H (tritium), ¹²⁵I (iodine-125) and ¹⁴C (carbon-14). Similarly, the compounds may be advantageously joined, covalently or noncovalently, directly or through a linker molecule, to a wide variety of other compounds, which may provide prodrugs or function as carriers, labels, adjuvants, coactivators, stabilizers, etc. Such labeled and joined compounds are contemplated within the present invention.

### Analysis of the Compounds

The subject compounds and compositions were demonstrated to have pharmacological activity in in vitro and in vivo assays, e.g., they are capable of specifically modulating a cellular physiology to reduce an associated pathology or provide or enhance a prophylaxis.

Certain preferred compounds and compositions are capable of specifically inhibiting or suppressing cytomegalovirus infection. For the assessment of activity against human CMV, a method was used which is similar to that described in Kohler, et al. (1994) J. Virol. 68:6589-6597. Briefly, a recombinant human cytomegalovirus (HCMV) was made containing a marker gene (luciferase) under the control of the promoter for the late 28 kDa viral structural phosphoprotein pp28. Human foreskin fibroblast (HFF) cells were infected with the recombinant HCMV virus (moi (multiplicity of infection) 5), placed into 96-well plates, and cultured under standard cell-culture conditions. Compounds that were evaluated for anti-HCMV activity were added to the infected cells 20 h later. The level of luciferase expression was measured 24 h after treatment with the test compounds. The biological activity of the test compounds is described by their IC₅₀ values, the concentration of test compound that reduces recombinant HCMV late gene expression (represented by luciferase expression in the HFF culture) by 50% relative to control (vehicle-treated) infected cells. As an additional control, the cytotoxicity of test compounds on untreated HFF cells was also evaluated in cultured cell growth experiments.

Table 1 provides biological data for selected compounds from the examples below.

TABLE 1

| Compound | IC₅₀ (μM) |
|---|---|
| a | 0.8 |
| c | 0.1 |
| d | 0.02 |
| f | 6 |
| g | 0.8 |
| h | 0.3 |
| j | 0.01 |
| k | 1 |
| m | 2 |
| n | 0.4 |
| o | 2 |
| p | 0.3 |
| q | 3 |
| s | 3 |
| t | 10 |

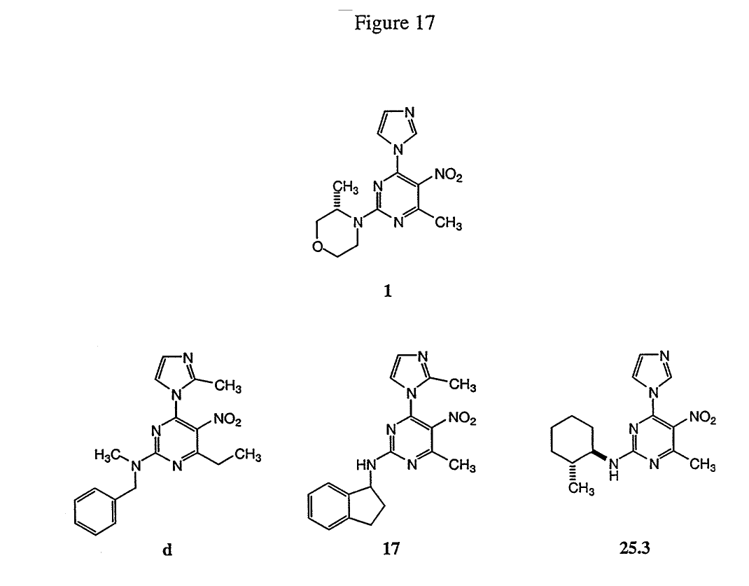

Evidence that the subject compounds modulate CMV DNA primase was obtained from a series of experiments. Typically 25 cm² flasks of HFF cells(1×10⁶ cells/flask) were mock-infected or infected with HCMV at a moi (multiplicity of infection) of 5 pfu/cell. After 1 h, the inoculum was removed and the cells were overlaid with fresh media containing the appropriate concentration of a tritiated compound, (³H)-d, (³H)-17 or (³H)-25.3 (see FIG. 17). The cells were incubated at 37° C. for 24, 48 or 96 h. The cells were then washed with PBS and scraped into 1 mL of PBS. The infected cells were centrifuged for 2 min. The supernatant was discarded and the cell pellet was resuspended and lysed in 300 μL of PBSA* (1% nonidet P40, 1% sodium deoxycholate, 10 nM PMSF, 10 nM TLCK, 10 nM TPCK, 1 mM EGTA, 10 nM approtinin in PBS). The samples were then sonicated for five 2-min. intervals, aliquotted and stored at −80° C. Fifty 1-μL samples were mixed with lamelli sample buffer (Biorad) and subjected to SDS electrophoresis in 10% or 4-20% gradient polyacrylamide gels. The electrophoretically separated radiolabeled proteins were transferred to nitrocellulose and exposed for 6 days to Ultrasensitive Fuji tritium detection plates and analyzed with a phosphoimager.

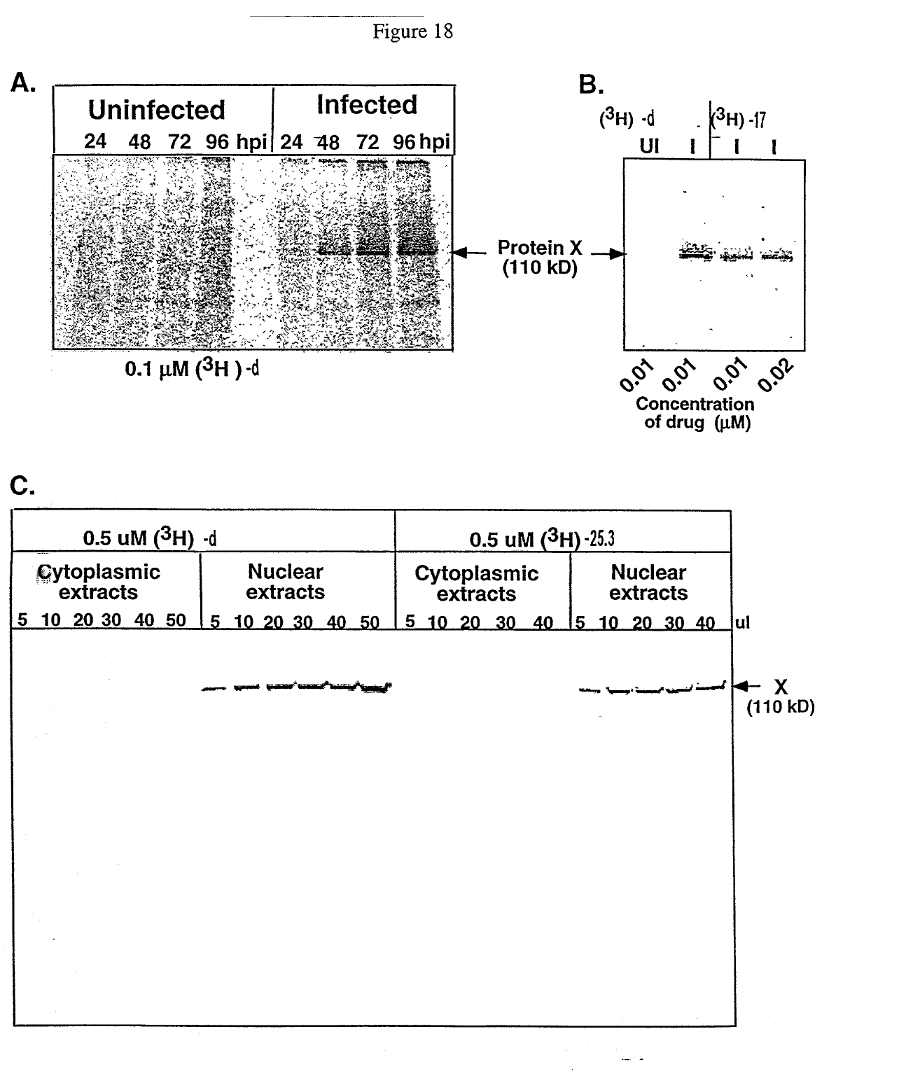

As shown in FIG. 18A, one specific protein (protein X) that migrated with an apparent molecular mass of approximately 110 kD was observed only in extracts derived from infected cells treated with (³H)-d and appeared at 48 h post-infection. The time of appearance of this protein is consistent with early gene expression in HCMV, which gives rise to the proteins involved in viral DNA replication.

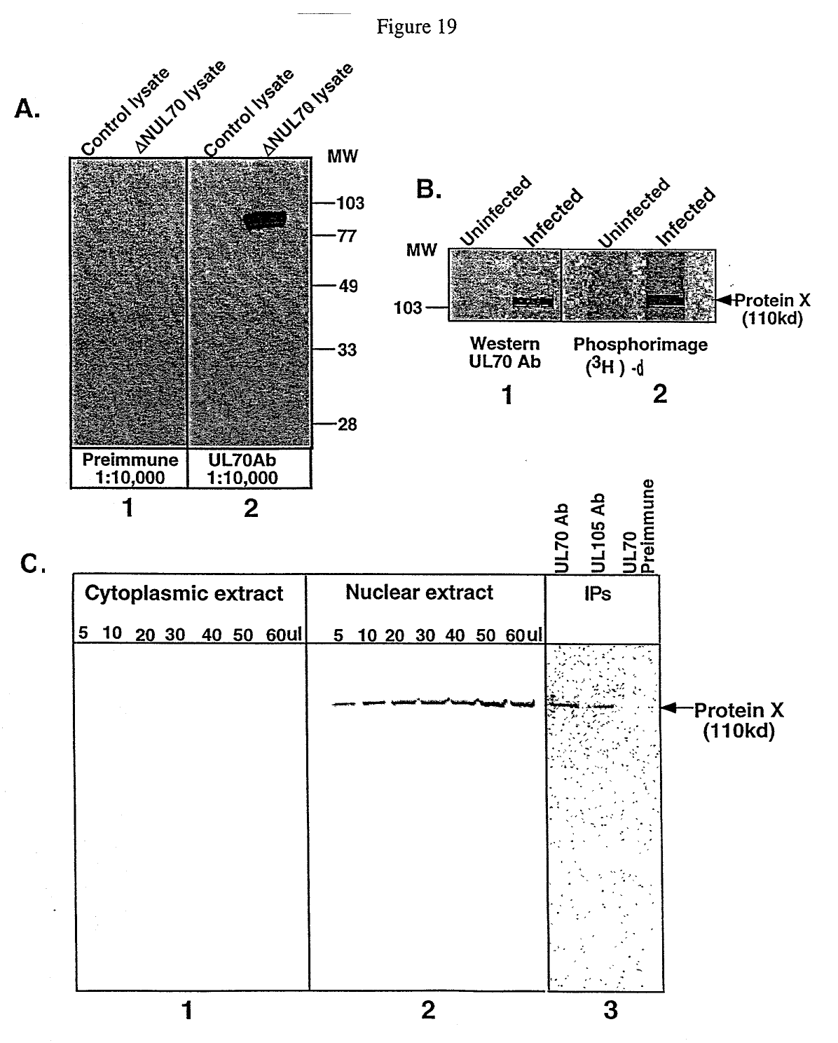

As shown in FIG. 19A, extracts from cells infected with either a baculovirus expressing an unrelated protein or baculovirus expressing the HCMV UL70 gene were subjected to SDS polyacrylamide electrophoresis in 4-20% gradient gels. The separated proteins were then transferred to nitrocellulose and probed with either preimmune serum or anti-HCMV UL70 peptide serum. Using the ECL detection system, a strong signal at ˜85 kD was observed with the immune serum only in extracts from insect (High Five) cells infected with baculovirus expressing UL70. The baculovirus-produced UL70 protein appeared as a doublet on SDS gels. Baculovirus UL70 protein lacks the first 100 amino acids at the N-terminus and thus, migrates faster than the full length UL70 protein in SDS polyacrylamide gels.

As shown in FIG. 19B (panel 1), UL70 can be detected in infected cells, and the specific UL70 antibody signal comigrated exactly with the (³H)-d-labeled 110 kD viral-specific protein X (FIG. 19B, panel 2).

Modification of CMV UL70 by the subject compounds was determined by generation of an HCMV mutant strain that is resistant to compound 1 and comparison of wild-type Towne sequences of the HCMV replication genes with sequences of the corresponding genes in the 1-resistant virus.

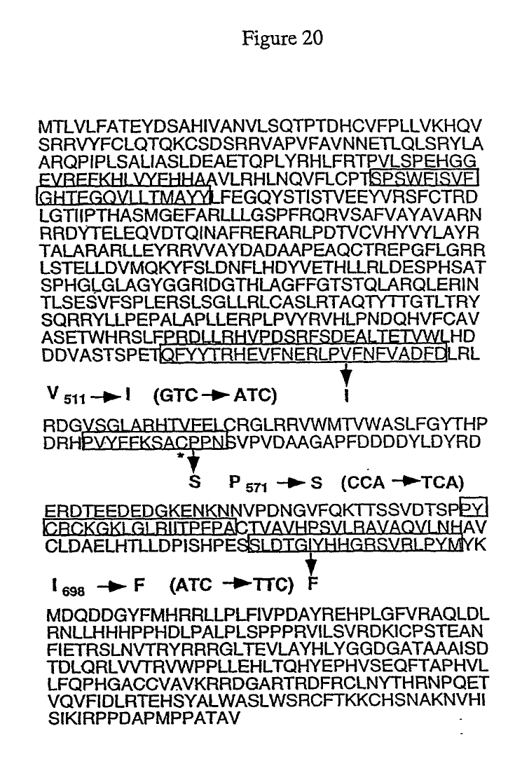

Eleven viral genetic loci have been shown to be required for HCMV DNA replication. To determine which of these genes is mutated in the mutant virus, DNA sequencing was performed. Wild type Towne sequences of the HCMV replication genes were compared with sequences of the corresponding genes in the 1-resistant virus. To detect bona fide point mutations, multiple pools of polymerase chain reaction (PCR) products were sequenced for each of the following genes (including 500 bp of flanking sequences): UL44 (DNA polymerase accessory factor), UL54 (DNA polymerase), UL57 (single-stranded DNA binding protein), UL102 (helicase-primase accessory factor), UL105 (helicase), UL101-UL102 (origin binding protein), UL112-113 loci (early proteins of unknown function), UL84 (unknown function) and UL70 (primase). The only changes identified were three point mutations in the UL70 gene encoding the HCMV primase, shown in FIG. 20. The presence of the three point mutations was confirmed by PCR amplification and sequencing of the UL70 genes from three independent preparations of the 1-resistant virus DNA.

All three point mutations detected are in the conserved primase domains of the UL70 protein. In particular, the cysteine residue at position 570 is completely conserved among all primase homologs identified in the herpes virus family. However, the adjacent residues are poorly conserved. The proline residue at position 571 is not a highly conserved residue. Site-specific mutagenesis of serine 571 back to proline in the presence of the V₅₁₁→I and I₆₉₂→F mutations could not rescue wild type viruses in the presence of high concentrations (1 μM) of an analog of compound 1. Furthermore, mutation of proline 571 to serine alone was not sufficient to confer a compound-resistant phenotype. Further mutagenesis studies in which V₅₁₁ and I₆₉₂ were restored individually to 1-resistant virus UL70 DNA were not sufficient to restore a resistant phenotype.

### Combinatorial Libraries

Combinatorial libraries of compounds that possess an electrophilic moiety capable of reacting with a thiol group can be screened for antiviral activity. Conventionally, new chemical entities with useful properties are generated by identifying a chemical compound (called a “lead compound”) with some desirable property or activity, e.g., antiviral activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et. al. (1994) J. Med. Chem. 37(9):1233-1251).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res. 37:487-493, Houghton et. al. (1991) Nature 354: 84-88), peptoid libraries (PCT Publication No WO 91/19735), encoded peptide libraries (PCT Publication WO 93/20242), random bio-oligomer libraries (PCT Publication WO 92/00091), benzodiazepine libraries (U.S. Pat. No. 5,288,514), libraries of diversomers, such as hydantoins, benzodiazepines and dipeptides (Hobbs et. al. (1993) Proc. Nat. Acad. Sci. USA 90:6909-6913), vinylogous polypeptide libraries (Hagihara et al. (1992) J. Amer. Chem. Soc. 114:6568), libraries of nonpeptidyl peptidomimetics with a Beta-D-Glucose scaffolding (Hirschmann et al. (1992) J. Amer. Chem. Soc. 114:9217-9218), analogous organic syntheses of small compound libraries (Chen et. al. (1994) J. Am. Chem. Soc. 116:2661), oligocarbamate libraries (Cho et al. (1993) Science 261:1303) and/or peptidyl phosphonate libraries (Campbell et al. (1994) J. Org. Chem. 59:658). See, generally, Gordon et al. (1994) J. Med. Chem. 37:1385-1401, nucleic acid libraries (see, e.g., Stratagene Corp.), peptide nucleic acid libraries (see, e.g., U.S. Pat. No. 5,539,083), antibody libraries (see, e.g., Vaughn et. al. (1996) Nature Biotechnology 14(3):309-314), and PCT/US96/10287), carbohydrate libraries (see, e.g., Liang et al. (1996) Science 274:1520-1522, and U.S. Pat. No. 5,593,853), and small organic molecule libraries (see, e.g., benzodiazepines, Baum (1993) C&EN Jan 18, page 33; isoprenoids, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky.; Symphony, Rainin, Woburn Mass.; 433A Applied Biosystems, Foster City Calif.; 9050 Plus, Millipore, Bedford, Mass.).

A number of well known robotic systems have also been developed for solution phase chemistries. These systems includes automated workstations like the automated synthesis apparatus developed by Takeda Chemical Industries, LTD. (Osaka, Japan) and many robotic systems utilizing robotic arms (Zymate II, Zymark Corporation, Hopkinton Mass.; Orca, Hewlett-Packard, Palo Alto Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see e.g., ComGenex, Princeton N.J.; Asinex, Moscow, Russia; Tripos, Inc., St. Louis Mo.; ChemStar, Ltd, Moscow, Russia; 3D Pharmaceuticals, Exton Pa.; Martek Biosciences, Columbia Md.; etc.).

### High Throughput Screening

High throughput assays for the presence, absence, quantification, or other properties of particular compounds are well known to those of skill in the art. Thus, for example, U.S. Pat. No. 6,043,038 discloses high throughput screening methods for modulators of primase activity. Such assays may be adapted to identify compounds capable of modifying CMV UL70 using functional protein. Preferred assays thus detect enhancement or inhibition of CMV DNA primase activity.

In addition, high throughput screening systems are commercially available (see e.g., Zymark Corp., Hopkinton Mass.; Air Technical Industries, Mentor Ohio; Beckman Instruments, Inc., Fullerton Calif.; Precision Systems, Inc., Natick Mass.; etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

The following examples are offered by way of illustration and not by way of limitation.

### EXAMPLES

¹H-NMR spectra were recorded on a Varian Gemini 400 MHz NMR spectrometer. Significant peaks are tabulated in the order: number of protons, multiplicity (s, singlet; d, doublet; t, triplet; q, quartet; m, multiplet; br s, broad singlet) and coupling constant(s) in Hertz. Electron Ionization (EI) mass spectra were recorded on a Hewlett Packard 5989A mass spectrometer. Mass spectrometry results are reported as the ratio of mass over charge (m/z), followed by the relative abundance of each ion (in parentheses).

### Example 1

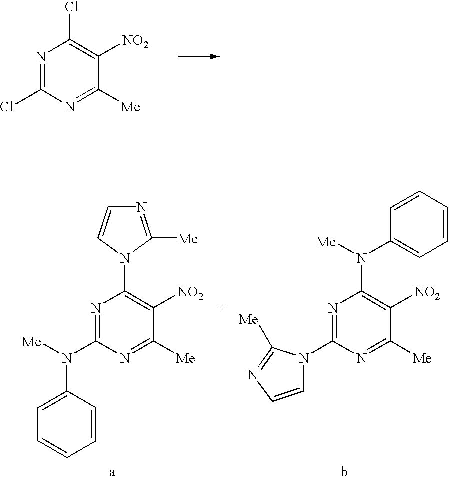

2-(N-methylanilino)-4-(2-methylimidazolyl)-6-methyl-5-nitropyrimidine (a) and an isomer 4-(N-methylanilino)-2-(2-methylimidazolyl)-6-methyl-5-nitropyrimidine (b). To a stirred cold (−78° C.) solution of 2,4-dichloro-6-methyl-5-nitropyrimidine (2.25 g, 10.8 mmol, 1.0 equiv.) in THF (15 mL) was added 2-methylimidazole (977 mg, 11.9 mmol, 1.1 equiv.) in a solution of THF (15 mL) dropwise. After 1 h, the dry ice bath was replaced with a water ice bath and stirring was continued for an additional 2 h and 15 min. At this time N-methylaniline (4.6 mL, 43.2 mmol, 4.0 equiv.) was added. The reaction solution was stirred 1 h and 15 min. at −78° C. and at room temperature overnight. At this time the solvent was removed and the residue was diluted with dichloromethane and washed three times with 0.1M HCl and three times with saturated aqueous NaCl solution. The organic phase was evaporated and the residue was purified by chromatography on silica gel (1:1 hexane/diethyl ether, 1% AcOH as eluant) to provide 209 mg of the target compound a (6%) along with an isomer (400 mg) and b (104.8 mg).

(a) ¹H NMR (400 MHz) (CD₃OD): δ2.26 (3H, br s); 2.58 (3H, br s); 3.61 (3H, s); 6.88 (1H, s); 7.02 (2H, d); 7.31-7.34 (3H, m); 7.43-7.48 (2H, m). Anal. calcd. for C₁₆H₁₆N₆O₂: C, 59.25; H, 4.97; N, 25.91. Found C, 59.16; H, 4.95; N, 25.86.

(b) ¹H NMR (400 MHz) (CDCl₃): δ2.40 (3H, s); 2.80 (3H, s); 3.55 (3H, s); 6.95 (1H, s); 7.13 (2H, m); 7.30-7.39 (3H, m); 7.86 (2H, s).

### Example 2

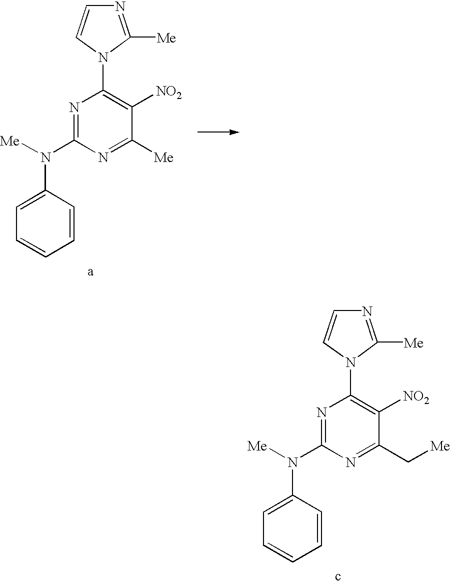

2-(N-methylanilino)-4-(2-methylimidazolyl)-6-ethyl-5-nitropyrimidine (c). To a stirred, cold (−78° C.) solution of a (54.4 mg, 0.168 mmol, 1.0 equiv.) in THF (1.0 mL) was added LiN(SiMe₃)₂, (0.20 mL, 0.201 mmol, 1.2 equiv., of a 1.0M/THF solution) dropwise. After stirring for 10 min., MeI (0.105 mL, 1.68 mmol, 10 equiv.) was added dropwise. The reaction was kept at −78° C. for 40 min. and stirred for an additional 4 h at 0° C. A small portion of acetic acid (0.25 mL) was poured into the flask and the brown residue was evaporated to dryness. The residue was then dissolved in dichloromethane and washed three times with saturated aqueous NaCl solution and the organic phase was evaporated to dryness to provide a crude yellow oil.

Purification was carried out by column chromatography on silica gel with 1:1 hexane/diethyl ether, 1% AcOH, 3% MeOH as eluant, to provide 21.4 mg of the desired product (37%).

(c) ¹H NMR (400 MHz) (CD₃OD): δ1.29 (3H, br s); 2.28 (3H, br s); 2.86 (2H, br s); 3.63 (3H, s); 6.89 (1H, s); 7.02 (1H, s); 7.30-7.39 (3H, m); 7.42-7.49 (2H, m). MS ESI m/z (relative intensity): M+H, 339.2 (100); M+Na, 361.1 (15).

### Example 3

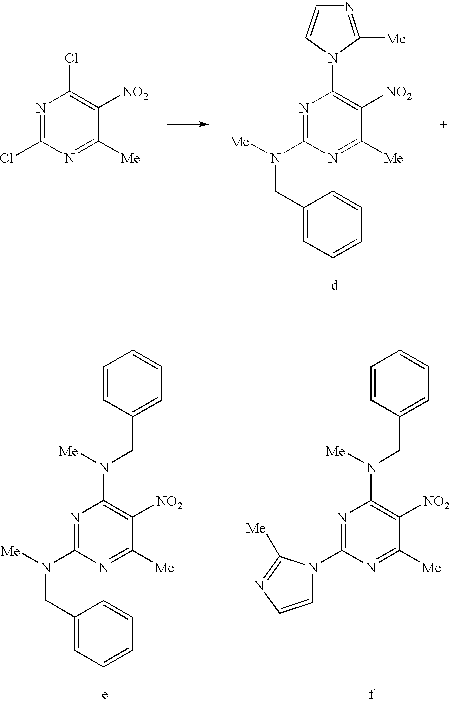

2-(N-benzylmethylamino)-4-(2-methylimidazolyl)-6-methyl-5-nitropyrimidine (d), 2,4-Bis-(N-benzylmethylamino)-6-methyl-5-nitropyrimidine (e) and 4-(N-benzylmethylamino)-2-(2-methylimidazolyl)-6-methyl-5-nitropyrimidine (f). To a stirred, cold (−78° C.) solution of 2,4-dichloro-6-methyl-5-nitropyrimidine (187.7 mg, 0.90 mmol, 1.0 equiv.) in THF (2.25 mL) and EtOH (2.25 mL) was added 2-methylimidazole (148 mg, 1.80 mmol, 2.0 equiv.) in a solution of EtOH (2.25 mL) dropwise. After 45 min., the dry ice bath was replaced with a water ice bath and the mixture was stirred for an additional 2 h 15 min. At this time N-methylbenzylamine (0.465 mL, 3.60 mmol, 4.0 equiv.) was added. After stirring for 2 h and 40 min., the solvents were removed by evaporation. The residue was diluted with dichloromethane and washed three times with 0.1 M HCl and three times with saturated aqueous NaCl solution. Solvent was removed from the organic phase and the residue was purified by chromatography on silica gel (1:1 hexane/diethyl ether, 1% AcOH, as eluant) to provide d (32 mg), e (116.3 mg) and f (104.8 mg).

(d) ¹H NMR (400 MHz) (CDCl₃): δ2.30 (1.5H, s); 2.53 (1.5H, s); 2.57 (1.5H, s), 2.57 (1.5H, s); 2.59 (1.5H, s); 3.15 (1.5H, s); 3.27 (1.5H, s); 4.88 (1H, s); 4.97 (1H, s); 6.87 (0.5H, s); 6.90 (0.5H, s); 6.96 (0.5H, s); 6.99 (0.5H, s); 7.16 (1H, d); 7.24-7.37 (4H, m). MS ESI m/z (relative intensity): M+H, 339.2 (100); M+Na, 361.1 (8)

(e) ¹H NMR (400 MHz) (CDCl₃): δ2.49 (3H, s); 2.79 (3H, s); 2.90-3.20 (3H, br humps); 4.70-4.88 (4H, br humps); 7.12-7.35 (10H, br humps). MS ESI m/z (relative intensity): M+H, 378.2 (100); M+Na, 400.1 (15)

(f) ¹H NMR (400 MHz) (CDCl₃): δ2.52 (3H, s); 2.67 (3H, s); 2.90 (3H, s); 4.92 (2H, s); 6.89 (1H, s); 7.20 (2H, d); 7.28-7.35 (3H, m); 7.74 (1H, s). MS ESI m/z (relative intensity): M+H, 339.2 (100).

### Example 4

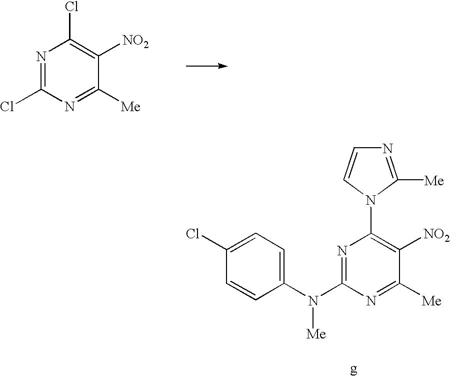

2-(N-methyl-4-chloroanilino)-4-(2-methylimidazolyl)-6-methyl-5-nitropyrimidine (g). To a stirred, cold (−78° C.) solution of 2,4-dichloro-6-methyl-5-nitropyrimidine (207.5 mg, 1.0 mmol, 1.0 equiv.) in THF (2.25 mL) and EtOH (2.25 mL) was added 2-methylimidazole (164 mg, 2.00 mmol, 2.0 equiv.) in a solution of EtOH (2.25 mL) dropwise. After 45 min., the dry ice bath was replaced with a water ice bath and stirring was continued for an additional 2 h and 15 min. 4—Chloro-N-methylaniline (0.485 mL, 4.0 mmol, 4.0 equiv.) was then added and the reaction solution was stirred for 2 h and 40 min. Solvent was removed by evaporation and the residue was diluted with dichloromethane, washed three times with 0.1 M HCl, three times with saturated aqueous NaCl solution and dried over MgSO₄. Solvent was removed from the organic phase and the residue was purified by silica gel chromatography (1:1 hexane/diethyl ether, 1% AcOH as eluant) to provide g (55.9 mg, 15.6%).

(g) ¹H NMR (400 MHz) (CD₃OD): δ2.30 (3H, br s); 2.57 (3H, br s); 3.59 (3H, s); 6.91 (1H, s); 7.02 (1H, s); 7.36 (2H, d); 7.44 (2H,d). MS ESI m/z (relative intensity): M+H, 359.1 (100).

### Example 5

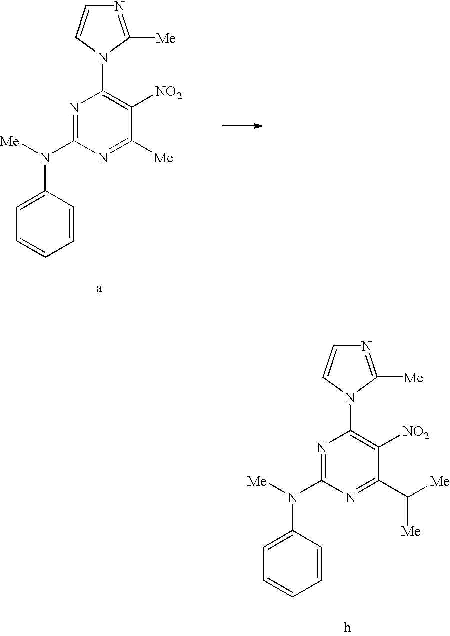

2-(N-methylanilino)-4-(2-methylimidazolyl)-6-isopropyl-5-nitropyrimidine (h). To a stirred, cold (−78° C.) solution of a (38.6 mg, 0.119 mmol, 1.0 equiv.) in THF (0.5 mL) was added NaH (9.5 mg, 0.24 mmol, 2.0 equiv., 60% in oil). After stirring for 15 min., MeI (0.074 mL, 1.19 mmol, 10 equiv.) was added. The reaction was kept at −78° C. for 2 h, then stirred an additional 2.5 h at 0° C. A small portion of acetic acid (0.25 mL) was poured into the flask and the brown mixture was evaporated to dryness. The residue was dissolved into dichloromethane, washed three times with water and three times with saturated aqueous NaCl solution. Solvent was removed from the organic phase and the product was purified by silica gel chromatography (1:1 hexane/diethyl ether, 1% AcOH as eluant) to provide the target compound (13.3 mg 33%).

(h) ¹H NMR (400 MHz) (CDCl₃): δ1.20-1.35 (6H, m); 2.29 (3H, br s); 3.24 (1H, m); 3.62 (3H, s); 4.92 (2H, s); 6.89 (1H, br s); 7.03 (1H, br s); 7.30-7.40 (3H, m); 7.71-7.48 (2H, m). MS ESI m/z (relative intensity): M+H, 353.1 (100).

### Example 6

### 2-(N-benzylmethylamino)-4-(2-methylimidazolyl)-6-ethyl-5-nitropyrimidine (j)

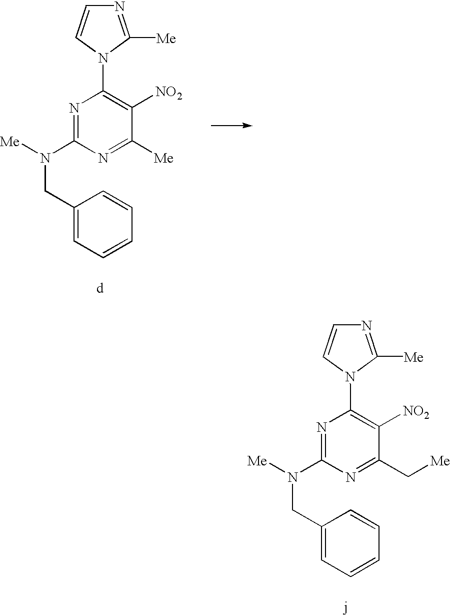

2-(N-methylanilino)-4-(2-methylimidazolyl)-6-isopropyl-5-nitropyrimidine (j). To a stirred, cold (−78° C.) solution of d (57.7 mg, 0.170 mmol in THF (0.5 mL) was added LiN(SiMe₃)₂, (0.17 mL, 0.17 mmol, 1.0 equiv., 1.0M/THF) dropwise. After stirring for 10 min., MeI (0.106 mL, 1.70 mmol, 10 equiv.) was added dropwise. The reaction was kept at −78° C. for 2 h and then stirred for an additional 3 h at 0° C. A small portion of acetic acid (0.25 mL) was poured into the flask and the brown mixture was evaporated to dryness. The residue was dissolved into dichloromethane, washed three times with water, three times with saturated aqueous NaCl solution and the organic phase was evaporated to dryness. The target compound was obtained following silica gel chromatography (1:1 hexane/diethyl ether, 1% AcOH, 3% MeOH as eluant). Yield: 30.3 mg (50.4%).

(j) ¹H NMR (400 MHz) (CD₃OD): δ1.26-1.41 (3H,m); 2.21 (1.5H,s); 2.45 (1.5H, s); 2.86-2.94 (2H, m); 3.22 (1.5H, s); 3.35 (1.5H, s); 4.93 (1H, s); 5.05 (1H,s); 6.91 (0.5H, s); 6.94 (0.5H, s); 7.07 (0.5H, s); 7.12 (0.5H, s); 7.23-7.38 (5H, m). MS ESI m/z (relative intensity): M+H, 353.1 (100).

### Example 7

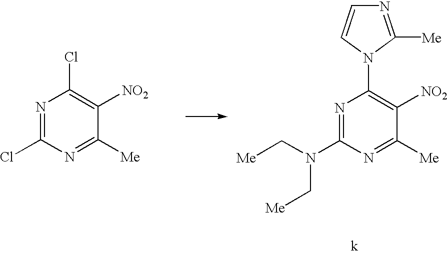

2-(N,N-diethylamino)-4-(2-methylimidazolyl)-6-methyl-5-nitropyrimidine (k). To a cooled (−78° C.) solution of 2,4-dichloro-6-methyl-5-nitropyrimidine (208 mg, 1.0 mmol, 1.0 equiv. in 2 mL each of EtOH and THF) was added 2.0 equiv. of 2-methylimidazole in 2 mL of EtOH. The resulting mixture was stirred for 1 hr at −78° C., then for 2 hr at 0° C. Diethylamine (0.413 mL, 4.0 equiv.) was added dropwise and the reaction was stirred overnight. The resulting mixture was diluted with dichloromethane, washed with 0.1 N HCl, saturated NaCl, dried (MgSO₄), and filtered. Solvent was removed by evaporation and the residue was purified by silica gel chromatography to provide 35 mg of the target compound k.

(k) ¹H NMR (400 MHz, CDCl₃): δ1.15-1.23 (3H, m); 2.48 (3H, s); 2.53 (3H, s); 3.59-3.60 (2H, q); 3.68-3.70 (2H, q); 6.86 (1H, s); 6.95 (1H, s). MS ESI m/z (relative intensity): M+H, 291.2 (100).

In a similar manner, the following compounds were prepared using the indicated amine in place of diethylamine. Each was obtained as a yellow oil.

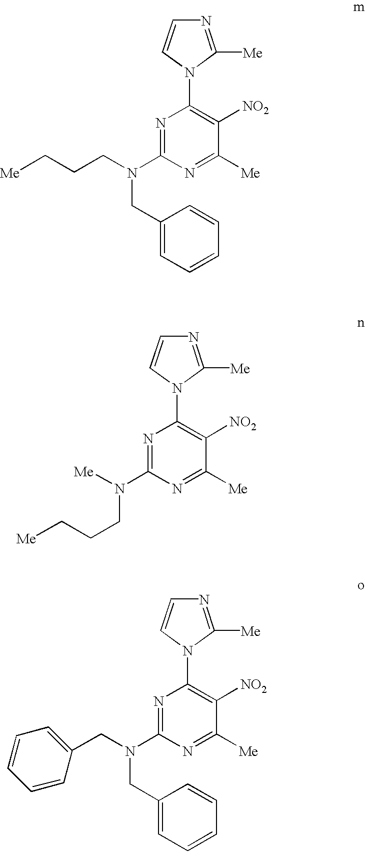

2-(N-benzylbutylamino)-4-(2-methylimidazolyl)-6-methyl-5-nitropyrimidine Compound m (N-butylbenzylamine) (m). 40 mg. ¹H NMR (400 MHz, CDCl₃): δ0.86-0.95 (3H, m); 1.23-1.38 (2H, m); 1.51-1.68 (2H, m); 2.52 (3H, m); 3.52 (2H, t); 4.83 (1H, s); 6.80 (1H, s); 6.92 (1H, s); 7.13 (2H, d); 7.26-7.31 (3H, m). MS ESI m/z relative intensity: M+H, 381.2 (100).

2-(N-methylbutylamino)-4-(2-methylimidazolyl)-6-methyl-5-nitropyrimidine Compound n (N-methylbutylamine) (n). 68 mg. ¹H NMR (400 MHz, CDCl₃): δ0.95 (3H, t); 1.32 (2H, m); 2.51 (3H, br s); 2.55 (3H, s); 3.15-3.24 (3H, d); 3.58-3.72 (2H, t); 6.85 (1H, s); 6.95 (1H, s). MS ESI m/z (relative intensity) M+H, 305.4 (100).

2-(N,N-dibenzylamino)-4-(2-methylimidazolyl)-6-methyl-5-nitropyrimidine Compound o (Dibenzylamine) (o). 20 mg. ¹H NMR (400 MHz, CDCl₃): δ2.53 (3H, br s); 2.55 (3H, br s); 4.81 (2H, s); 4.96 (2H,s); 6.85 (1H, s); 6.95 (1H, s). MS ESI m/z (relative intensity) M+H, 415.6 (100).

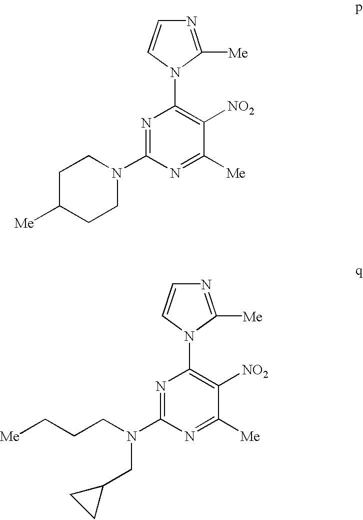

Compound p (4-methylpiperidine). 45 mg. ¹H NMR (400 MHz, CDCl₃): δ1.12-1.16 (3H, m); 2.46 (3H, s); 2.51 (3H, s); 3.40-3.47 (8H, m); 6.84 (1H, s); 6.99 (1H, s). MS ESI m/z (relative intensity): M+H, 317.1 (100).

Compound q (N-(cyclopropylmethyl)butylamine). 41 mg. ¹H NMR (400 MHz, CDCl₃): δ0.23-0.64 (4H, m); 0.89-0.93 (3H, m); 1.18 (1H, t); 1.59-1.73 (2H, m); 2.49-2.51 (3H, d); 2.54-2.55 (3H, d); 3.46-3.58 (2H, m). MS ESI m/z (relative intensity): M+H, 331.2 (100).

### Example 8

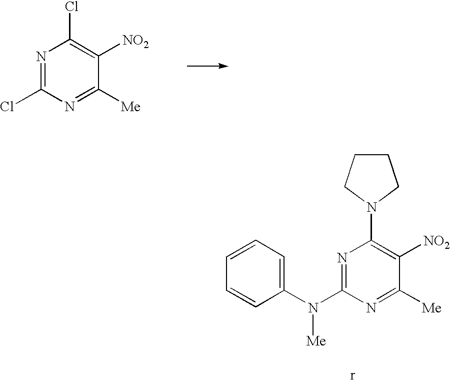

2-(N-methylanilino)-4-pyrrolidino-6-methyl-5-nitropyrimidine (r). To a cooled (−78° C.) solution of 2,4-dichloro-6-methyl-5-nitropyrimidine (208 mg, 1.0 mmol, 1.0 equiv. in 2 mL each of EtOH and THF) is added 1.1 equiv. of pyrollidine in 1.0 mL of EtOH. The resulting solution is stirred for 1 hr at −78° C., then for 2 hr at 0° C. N-methylaniline (0.432 mL, 4.0 equiv.) is added dropwise and the reaction is stirred overnight. The resulting mixture is diluted with dichloromethane, washed with 0.1 N HCl, saturated NaCl, dried (MgSO₄), and filtered. Solvent is removed by evaporation and the residue is purified by chromatography to provide the target compound r.

### Example 9

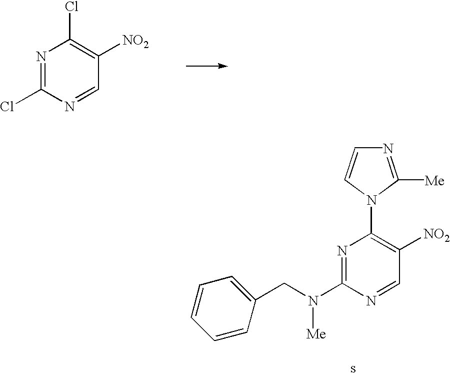

2-(N-Methylbenzylamino)-4-(2-methylimidazolyl)-5-nitropyrimidine (s). To a solution of 2,4-dichloro-5-nitropyrimidine (200 mg, 1 mmol) in dioxane (5 mL) at 80° C. was added 2-methylimidazole (85 mg, 1 mmol) and N-methylbenzylamine (133 μL, 1 mmol). The solution was stirred overnight at 80° C., cooled, and directly chromatographed (1/1 hexane diethyl ether) to yield product.

(s) ¹H NMR (400 MHz) (CD₃OD): δ3.09 (s, 1.5H), 3.17 (s, 1.5H), 3.18 (s, 1.5H), 4.5-4.8 (m, 2H), 7.2-7.5 (m, 8H).

### Example 10

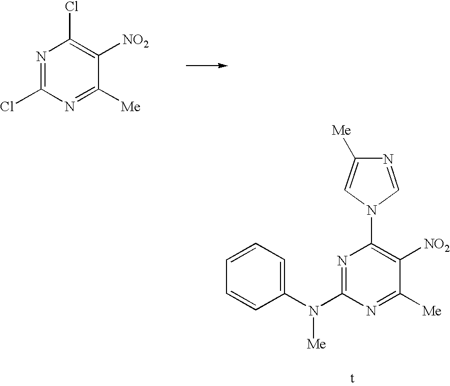

2-N-Methylanilino)-4-(4-methylimidazolyl)-5-nitro pyrimidine (t). To a solution of 2,4-dichloro-6-methyl-5-nitropyrimidine (150 mg, 0.72 mmol) in dioxane (5 mL) at 80° C. was added 4-methylimidazole (60 mg, 0.72 mmol) and N-methylaniline (77 mg, 0.72 mmol). The solution was stirred overnight at 80° C., cooled, and directly chromatographed (1/1 hexane diethyl ether) to yield product.

(t) ¹H NMR (400 MHz) (CD₃OD): δ2.37 (s, 3H), 2.74 (s, 3H), 3.30 (s, 3H), 7.25-7.55 (m, 5H), 7.75 (s, 1H), 9.31 (s, 1H).

### Example 11

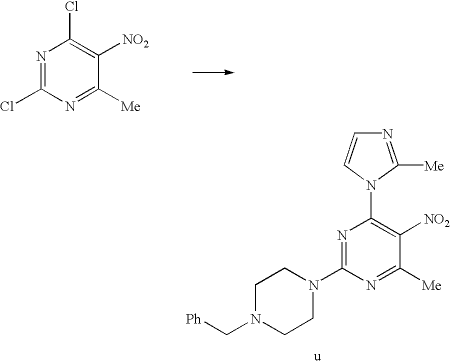

2-(4-Benzylpiperazine)-4-(2-methylimidazolyl)-6-methyl-5-nitro pyrimidine (u). To a solution of 2,4-dichloro-6-methyl-5-nitropyrimidine (175 mg, 0.84 mmol) in dioxane (5 mL) at 80° C. was added 2-methylimidazole (85 mg, 0.84 mmol) and 1-benzylpipirazine (148 μL, 0.84 mmol). The solution was stirred overnight at 80° C., cooled, and directly chromatographed (1/1 hexane diethyl ether) to yield product.

(u) ¹H NMR (400 MHz) (CD₃OD): δ2.42 (s, 3H), 2.60 (s, 3H), 3.38 (br s, 4H), 3.80 (br s, 4H), 4.38 (s, 2H), 7.30-7.55 (m, 7H). MS ESI 347 m/e (relative intensity): M+H, 348.0 (100).

### Example 12

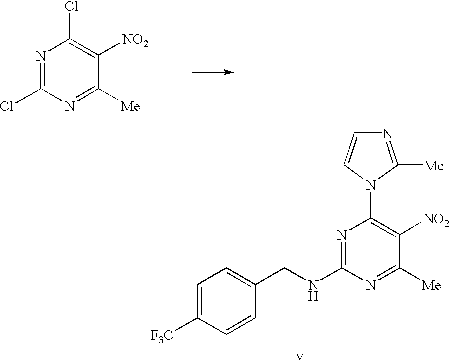

2-(4-trifluoromethylbenzylamino)-4-(2-methylimidazol-1-yl)-6-methyl-5-nitropyrimidine (v). To a stirred mixture of 2-chloro-4-hydroxy-6-methyl-5-nitropyrimidine (300 mg, 1.58 mmol, 1.0 equiv.) in absolute ethanol (20 mL) was added 4-(trifluoromethyl)benzylamine (540 mg, 3.08 mmol, 1.95 equiv.), and sodium acetate (130 mg, 1.58 mmol, 1.0 equiv.). The mixture was slowly heated and the resulting solution refluxed for 22 h. The mixture was then cooled and ethanol was removed in vacuo. The oily residue was dissolved in ethyl acetate and washed three times with 1M HCl, three times with saturated NaCl solution, then dried over MgSO₄. Removal of solvent provided a crude yellow solid intermediate which was dried under vacuum then dissolved in 4 mL of POCl₃ with heating (95-100° C.) for 0.5 h. The POCl₃ was removed by rotary evaporation and the crude brown product was purified using chromatography (1:1 hexane/dichloromethane) to provide a chloropyrimidine intermediate (313 mg), which was carried on directly without additional purification.

To a stirred solution of the above chloropyrimidine (150 mg, 0.43 mmol, 1.0 equiv.) in acetonitrile (2.5 mL) was added methylimidazole (142 mg, 1.7 mmol, 4.0 equiv.). The resulting mixture was heated to reflux for 5 h, cooled, and the solvent removed by rotary evaporation. The residue was dissolved in ethyl acetate, washed with 0.1M HCl, H₂O, brine and dried over MgSO₄ to give a crude yellow solid following removal of solvent. The solid was purified using chromatography with 2.5% MeOH/dichloromethane to give a yellow oil. A solid product was obtained by precipitation from dichloromethane and hexane. Yield: 152.3 mg, 51% from the starting 2-chloro-4-hydroxy-6-methyl-5-nitropyrimidine.

¹H NMR (400 MHz) CDCl₃ δ2.28 (1.5H, s); 2.42 (1.5H, s); 2.55 (1.5H, s); 2.58 (1.5H, s); 4.71 (1H, d); 4.80 (1H, d); 6.67 (0.5H, br s); 6.80 (0.5H,br s); 6.88 (1H, d); 6.96 (1H, s); 7.41 (1H, d); 7.49 (1H, d); 7.62 (2H, d). MS ESI m/z (relative intensity: M+H 392.9 (100).

### Example 13

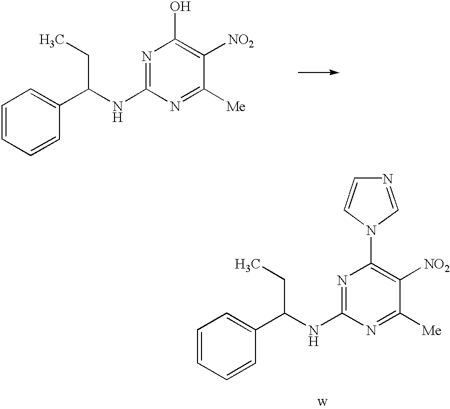

2-(1-phenyl-1-propylamino)-4-(imidazol-1-yl)-6-methyl-5-nitropyrimidine (w) using an alternate procedure for the addition of an imidazole group to the pyrimidine nucleus: To a stirred solution of 2-(1-phenylpropylamino)-4-hydroxy-6-methyl-5-nitropyrimidine (78 mg, 0.270 mmol, 1.0 equiv., prepared in a manner similar to that in Example 12 above) in pyridine (1 mL) was added trifluoroacetic anhydride (115 μL, 0.812 mmol, 3.0 equiv.). The mixture was stirred for 15 min., then imidazole (184 mg, 2.70 mmol, 10 equiv.) was added, and the mixture allowed to stir overnight. Pyridine was removed by rotary evaporation and the dark residue was dissolved in ethyl acetate and washed with 0.1M HCl followed by brine. The crude solid obtained after removal of solvent was purified by chromatography 2.5% MeOH/CH₂Cl₂ to give 36.1 mg (42%) of the title compound.

¹H NMR (400 MHz) CDCl₃ δ0.99 (3H, m); 1.73-2.02 (2H, m); 2.48 (3H, s); 4.81 (0.66H, dd); 5.07 (0.33H, dd); 6.16 (0.66H, d); 7.02 (0.33H, d); 7.08-7.12 (2H,m); 7.25-7.38 (5H, m); 7.89 (0.66H, s); 8.18 (0.33H, s). MS ESI m/z (relative intensity: M+H 339.2 (100).

### Example 14

This example illustrates the synthesis of pyrimidine derivatives having an alkoxy group in the 2-position, exemplified by 2-(propyloxy)-4-(2-methylimidazol-1yl)-6-methyl-5-nitropyrimidine (x).

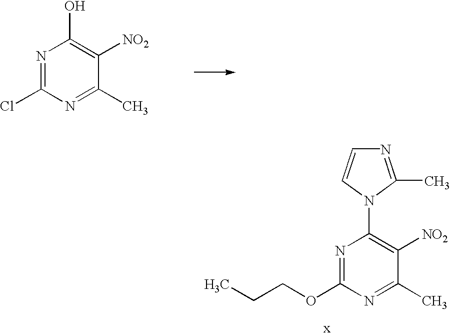

To a flask charged with n-propanol (5 mL) was added NaH (128 mg, 3.19 mmol, 2.0 equiv., 60% in oil) and the mixture was stirred under N₂ for 10 min. The resulting solution was transferred via cannula into a flask containing a solution of 2-chloro-4-hydroxy-6-methyl-5-nitropyrimidine (302 mg, 1.60 mmol, 1.0 equiv.) in n-propanol (5 mL). The resulting mixture was heated in an oil bath at 100° C. for 1 h, poured into a separatory funnel containing dilute HCl and extracted with dichloromethane. The organic phase was separated and washed with water, brine and dried over MgSO₄ to give a crude solid (yield 297 mg) after removal of solvent. The crude solid was heated in neat POCl₃ (3 mL) for 6 min. at 85-90° C., cooled on ice, and the POCl₃ was removed in vacuo. The chloropyrimidine intermediate was purified via chromatography to provide 117 mg of the intermediate which was converted to the title compound using methods described in Example 12. The product was obtained as a yellow oil (191 mg, 43% from 2-chloro-4-hydroxy-6-methyl-5-nitropyrimidine).

¹H NMR (400 MHz) CDCl₃ δ1.04 (3H, t); 1.86 (2H, dq); 2.52 (3H, s); 2.61 (3H, s); 4.38 (2H, t); 6.90 (1H, d); 6.98 (1H, d). MS ESI m/z (relative intensity: M+H 278.1 (100)

### Example 15

The compounds listed in Table 2 were prepared using the procedures outlined in Examples 12-13. Compounds were tested in the CMV assay described above and exhibited the following levels of activity: +, IC₅₀>500 nM; ++, 100 nM<IC₅₀<500 nM; +++, IC₅₀<100 nM.

TABLE 2

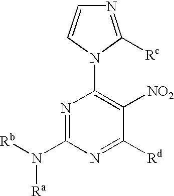

| | | | | m/z (m + 1) | |
|---|---|---|---|---|---|
| R^{a} | R^{b} | R^{c} | R^{d} | or mp (° C.) | IC₅₀ |
| 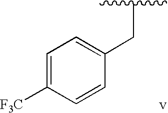 | H | Me | Me | 392.9 | ++ |
| 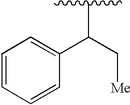 | H | Me | Me | 353.1 | ++ |
| 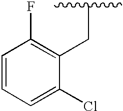 | H | Et | Me | 391.1 | ++ |
| 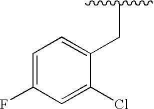 | H | Et | Me | 406.9 | ++ |
| 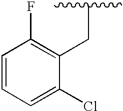 | H | Me | Me | 377.1 | ++ |
| 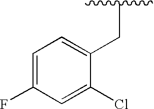 | H | Et | Me | 391.1 | ++ |
| 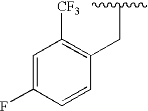 | H | Me | Me | 411.1 | ++ |
| 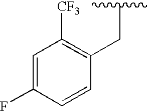 | H | Et | Me | 425.1 | ++ |
| 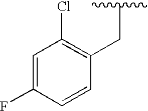 | H | Me | Me | 377.1 | ++ |
| 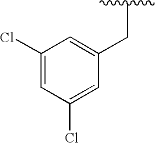 | H | Me | Me | 393.1 | ++ |
| 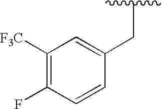 | H | Me | Me | 411.1 | +++ |
| 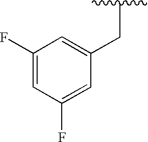 | H | Me | Me | 361.1 | +++ |
| 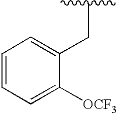 | H | Me | Me | 409.1 | ++ |
| 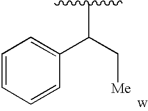 | H | H | Me | 339.2 | + |
| 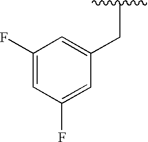 | H | H | Me | 347.1 | + |
| 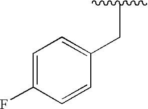 | H | Me | Me | 343.1 | ++ |
| 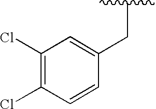 | H | Me | Me | 393.1 | ++ |
| 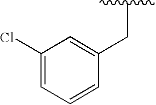 | H | Me | Me | 359.1 | +++ |
| 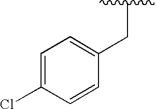 | H | Me | Me | 359.1 | ++ |
| 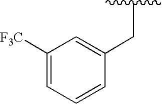 | H | Me | Me | 392.1 | +++ |
| 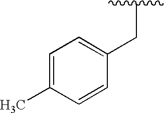 | H | Me | Me | 339.1 | + |
| 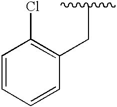 | H | Me | Me | 359.1 | +++ |
| 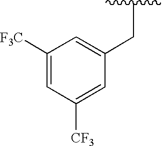 | H | Me | Me | 461.1 | + |
| 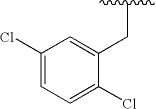 | H | Me | Me | 393.1 | +++ |
| 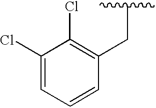 | H | Me | Me | 393.1 | ++ |
| 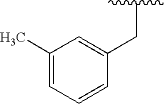 | H | Me | Me | 339.1 | ++ |
| 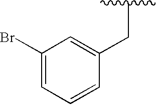 | H | Me | Me | 403.0 | +++ |
| 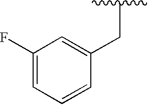 | H | Me | Me | 343.1 | ++ |
| 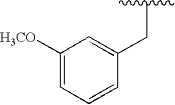 | H | Me | Me | 355.1 | ++ |
| 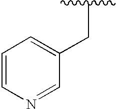 | H | Me | Me | 326.1 | + |
| 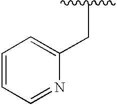 | H | Me | Me | 326.1 | ++ |
| 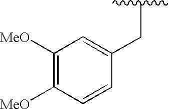 | H | Me | Me | 385.1 | ++ |
| 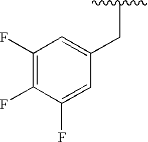 | H | Me | Me | 379.1 | ++ |
| 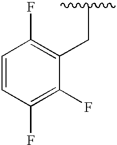 | H | Me | Me | 379.1 | ++ |
| 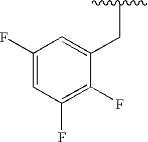 | H | Me | Me | 379.1 | ++ |
| 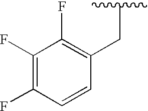 | H | Me | Me | 379.1 | ++ |
| 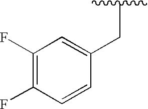 | H | Me | Me | 361.1 | ++ |
| 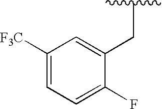 | H | Me | Me | 411.1 | +++ |
| 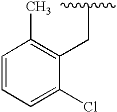 | H | Me | Me | 373.1 | ++ |
| 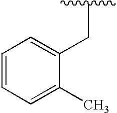 | H | H | Me | 325.1 | + |
| 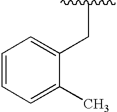 | H | Me | Me | 339.1 | ++ |
| 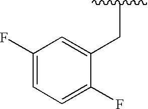 | H | Me | Me | 361.1 | ++ |

### Example 16

The compounds listed in Table 3 were prepared using the procedures outlined in Examples 12-14. Compounds were tested in the CMV assay described above and exhibited the following levels of activity: +, IC₅₀>500 nM.

TABLE 3

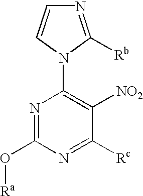

| | | | m/z (m + 1) | |
|---|---|---|---|---|
| R^{a} | R^{b} | R^{c} | or mp (° C.) | IC₅₀ |
| n-propyl (x) | Me | Me | 278.1 | + |
| n-propyl | H | Me | 264.1 | + |
| n-butyl | Me | Me | 292.2 | + |
| n-butyl | H | Me | 278.1 | + |
| phenethyl | H | Me | 326.1 | + |
| methyl | Me | Me | 250.1 | + |
| ethyl | Me | Me | 264.1 | + |
| benzyl | H | Me | 312.2 | + |
| 3-methoxy-1- | H | Me | 308.1 | + |
| butyl | | | | |
| 3-methoxy-1- | Me | Me | 322.3 | + |
| butyl | | | | |
| 3,3-dimethyl-1- | H | Me | 306.2 | + |
| butyl | | | | |
| 3,3-dimethyl-1- | Me | Me | 320.1 | + |
| butyl | | | | |

### Example 17

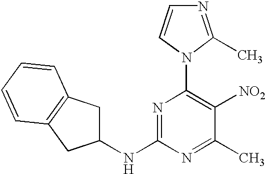

2-(2-indanamino)-4-(2-methylimidazoyl)-6-methyl-5-nitropyrimidine (17). 2-(2-Indanamino)-4-chloro-6-methyl-5-nitropyrimidine (56 mg, 0.184 mmol, 1.0 equiv) was dissolved in 2.0 mL EtOH followed by the addition of 38 mg of 2-methylimidazole (0.463 mmol, 2.5 equiv). The resulting yellow solution was placed in an 80° C. bath and allowed to stir for 24 h. The solution was then concentrated under reduced pressure. Purification by flash chromatography (SiO₂, 2% MeOH/CH₂Cl₂) gave 34 mg of the product as an amorphous yellow solid (0.096 mmol, 52%): mp 203-204° C.

¹H NMR (CDCl₃, 400 MHz, mixture of rotamers) δ7.28-7.13 (m, 5 H), 6.99 (s, 0.5 H), 6.96 (s, 0.5 H), 6.17 (d, J=7.9 Hz, 0.5 H), 6.06 (d, J=7.3 Hz, 0.5 H), 4.93 (m, 0.5 H), 4.73 (m, 0.5 H), 3.45-3.34 (m, 2 H), 2.94 (dd, J=4.8, 16.2 Hz, 1 H), 2.89 (dd, J=4.3, 16.0 Hz, 1 H), 2.71 (s, 1.5 H), 2.65 (s, 1.5 H), 2.63, s, 1.5 H), 2.53 (s, 1.5 H); MS: ESI(+) 351.2 (M+H⁺, rel. abund 100). Anal. calcd for C₁₈H₁₈N₆O₂: C, 61.70; H, 5.18; N, 23.99. Found: C, 61.08; H, 5.22; N, 23.57.

### Example 18

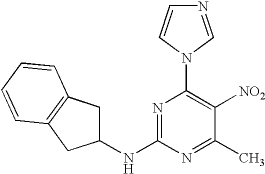

2-(2-indanamino)-4-imidazolyl-6-methyl-5-nitropyrimidine (18). 2-(2-Indanamino)-4-chloro-6-methyl-5-nitropyrimidine (66.8 mg, 0.219 mmol, 1.0 equiv) was dissolved in 2.0 mL EtOH followed by the addition of 37 mg of imidazole (0.543 mmol, 2.5 equiv). The yellow solution was heated to 80° C. for 18 h. The solution was then concentrated under reduced pressure and purified by flash chromatography (SiO₂, 2% MeOH/CH₂Cl₂) to give 52.1 mg of the product as an amorphous yellow solid (0.155 mmol, 71%): mp 177-178° C.

¹H NMR (CDCl₃, 400 MHz, mixture of rotamers) δ8.23 (s, 0.5 H), 8.16 (s, 0.5 H), 7.28-7.11 (m, 6 H), 6.09 (broad s, 0.5 H), 5.91 (d, J=7.2 Hz, 0.5 H), 4.93 (m, 0.5 H), 4.79 (m, 0.5 H), 3.40 (dd, J=7.0, 15.9 Hz, 2 H), 2.91 (dd, J=4.1, 15.8 Hz, 2 H), 2.56 (s, 1.5 H), 2.46 (s, 1.5 H); ); MS: ESI(+) 337.1 (M+H⁺, rel. abund 100). Anal. calcd for C₁₇H₁₆N₆O₂: C, 60.71; H, 4.79; N, 24.99. Found: C, 60.29; H, 4.89; N, 24.69.

### Example 19

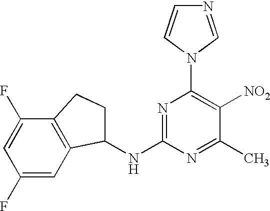

2-(4,6-difluoro-1-indanamino)-4-imidazolyl-6-methyl-5-nitropyrimidine (19). 2-(4,6-Difluoro-1-indanamino)-4-chloro-6-methyl-5-nitropyrimidine(56 mg, 0.164 mmol, 1.0 equiv) was dissolved in 2.0 mL EtOH followed by the addition of 28 mg imidazole (0.411 mmol, 2.5 equiv). The solution was heated to 80° C. for 23 h. The solution was then concentrated under reduced pressure and purified by flash chromatography (SiO₂, 2% MeOH/CH₂Cl₂) to give 35.5 mg of the product (0.095 mmol, 58%) as an amorphous yellow solid. mp 175-176° C.

¹H NMR (CDCl₃, 400 MHz, mixture of rotamers) δ8.09 (s, 0.5 H), 8.06 (s, 0.5 H), 7.26-7.10 (m, 2 H), 6.82 (dd, J=7.6, 11.6 Hz, 1 H), 6.72 (dd, J=8.8, 8.8 Hz, 1 H), 5.95 (broad s, 0.5 H), 5.82 (d, J=8.4 Hz, 0.5 H), 5.72 (m, 0.5 H), 5.56 (m, 0.5 H), 3.05 (m, 1 H), 2.87 (m, 1 H), 2.73 (m, 1 H), 2.55 (s, 1.5 H), 2.49 (s, 1.5 H), 1.98 (m, 1 H); ); MS: ESI(+) 373.1 (M+H⁺, rel. abund 100). Anal. calcd for C₁₇H₁₄F₂N₆O₂: C, 54.84; H, 3.79; N, 22.57. Found: C, 54.95; H, 3.76; N, 22.32.

### Example 20

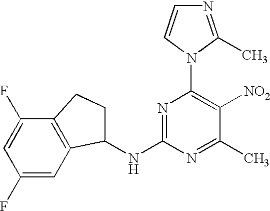

2-(4,6-difluoro-1-indanamino)-4-(2-methylimidazolyl)-6-methyl-5-nitropyrimidine (20). 2-(4,6-Difluoro-1-indanamino)-4-chloro -6-methyl-5-nitropyrimidine (56 mg, 0.164 mmol, 1.0 equiv) was dissolved in 2.0 mL EtOH followed by the addition of 34 mg 2-methylimidazole (0.414 mmol, 2.5 equiv) and the solution was heated to 80° C. with stirring for 26 h. The solution was then concentrated under reduced pressure and purified by flash chromatography (SiO₂, 2% MeOH/CH₂Cl₂) to give 42.6 mg of the product (0.110 mmol, 67%) as an amorphous yellow solid. mp 164-165° C.

¹H NMR (CDCl₃, 400 MHz, mixture of rotamers) δ6.98 (s, 1 H), 6.90 (s, 1 H), 6.81 (m, 1 H), 6.71 (m, 1 H), 5.87-5.81 (m, 1 H), 5.73 (m, 0.5 H), 5.54 (m, 0.5 H), 3.05 (m, 1 H), 2.82 (m, 1 H), 2.70 (m, 1 H), 2.60 (s, 1.5 H), 2.53 (s, 1.5 H), 2.51 (s, 1.5 H), 2.46 (s, 1.5 H), 1.98 (m, 1 H); ); MS: ESI(+) 387.1 (M+H⁺, rel. abund 100). Anal. calcd for C₁₈H₁₆F₂N₆O₂: C, 55.96; H, 4.17; N, 21.75. Found: C, 56.15; H, 4.59; N, 20.71.

### Example 21

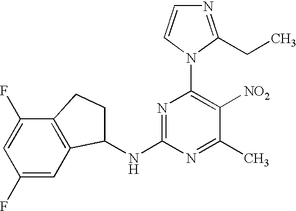

2-(4,6-difluoro-1-indanamino)-4-(2-ethylimidazolyl)-6-methyl-5-nitropyrimidine (21). 2-(4,6-Difluoro-1-indanamino)-4-chloro -6-methyl-5-nitropyrimidine (56 mg, 0.164 mmol, 1.0 equiv) was dissolved in 2.0 mL EtOH followed by the addition of 39 mg 2-ethylimidazole (0.406 mmol, 2.5 equiv) and the solution was heated to 80° C. for 23.5 h. The solution was then concentrated under reduced pressure and purified by flash chromatography (SiO₂, 2% MeOH/CH₂Cl₂) to give 39.6 mg of the product (0.099 mmol, 60%) as an amorphous yellow solid. mp 88-89° C.

¹H NMR (CDCl₃, 400 MHz, mixture of rotamers) δ7.02 (s, 1 H), 6.88 (s, 1 H), 6.81 (m, 1 H), 6.72 (m, 1 H), 5.85 (d, J=9.0 Hz, 0.5 H), 5.81-5.70 (m, 1 H), 5.55 (m, 0.5 H), 3.04 (m, 1 H), 2.86-2.64 (m, 4 H), 2.60 (s, 1.5 H), 2.53 (s, 1.5 H), 1.98 (m, 1 H), 1.29 (t, J=7.5 Hz, 3 H); MS: ESI(+) 401.1 (M+H⁺, rel. abund 100). Anal. calcd for C₁₉H₁₈F₂N₆O₂: C, 57.00; H, 4.53; N, 20.99. Found: C, 56.93; H, 4.50; N, 20.71.

### Example 22

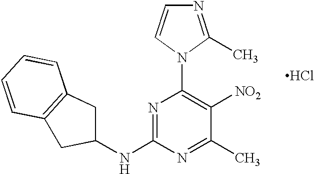

2-(2-indanamino)-4-(2-methylimidazoyl)-6-methyl-5-nitropyrimidinium hydrochloride (22). 2-(2-Indanamino)-4-chloro-6-methyl-5-nitropyrimidine (310 mg, 1.02 mmol, 1.0 equiv.) was dissolved in 7 mL EtOH followed by the addition of 600 mg 2-methylimidazole (7.31 mmol, 7.19 equiv.). The resulting yellow solution was then heated to 80° C. with magnetic stirring. After 24 h the solution was concentrated under reduced pressure and purified by flash chromatography (SiO_{2, 2}% MeOH/CH₂Cl₂) to give 303.6 mg of the free base as a yellow solid (0.867 mmol). The yellow solid was then dissolved in 3 mL anhydrous THF followed by the addition of 2 mL (8.0 mmol, 9.2 equiv.) of a 4.0 M solution of HCl in 1,4-dioxane. A precipitate was immediately formed, and the resulting slurry was allowed to stir for 10 min. The slurry was then concentrated under reduced pressure, taken up in 3 mL THF, and concentrated again. The resulting yellow solid was recrystallized from hot EtOAc to give 179 mg of the pyridinium hydrochloride as light yellow needles (0.462 mmol, 45%). mp 184-185° C.

¹H NMR (CD₃OD, 400 MHz, mixture of rotamers) δ7.76 (d, J=2.2 Hz, 0.5 H), 7.71 (d, J=2.2 Hz, 0.5 H), 7.64 (d, J=2.2 Hz, 0.5 H), 7.61 (d, J=2.2 Hz, 0.5 H), 7.22 (m, 2 H), 7.15 (m, 2 H), 4.92 (m, 0.5 H), 4.72 (m, 0.5 H), 3.41-3.31 (m, 1 H), 2.97 (m, 1 H), 2.73 (s, 1.5 H), 2.72 (s, 1.5 H), 2.68 (s, 1.5 H), 2.65 (s, 1.5 H). Anal. calcd for C₁₈H₁₈N₆O₂.HCl: C, 55.89; H, 4.95; N, 21.73; Cl, 9.16. Found: C, 55.89; H, 5.00; N, 21.56; Cl, 9.14.

### Example 23

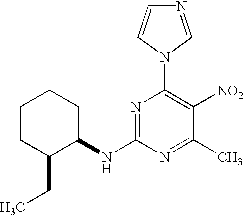

2-(syn-2-ethylcyclohexylamino)-4-imidazolyl-6-methyl-5-nitropyrimidine. 2-(syn-2-Ethylcyclohexylamino)-4-chloro-6-methyl-5-nitropyrimidine (58.6 mg, 0.196 mmol, 1.0 equiv.) was dissolved in 2.0 mL EtOH followed by the addition of 53 mg imidazole (0.778 mmol, 4.0 equiv.). The resulting yellow solution was then heated to 80° C. with magnetic stirring. After 20 h the solution was concentrated under reduced pressure and purified by flash chromatography (SiO₂, 2% MeOH/CH₂Cl₂) to give 39.5 mg of the product (0.120 mmol, 61%) as an amorphous yellow solid. mp 123-124° C.

¹H NMR (CDCl₃, 400 MHz, mixture of rotamers) δ8.22 (s, 0.5 H), 8.17 (s, 0.5 H), 7.39-7.27 (m, 2 H), 5.92 (d, J=7.8 Hz, 1 H), 4.57 (m, 0.5 H), 4.42 (m, 0.5 H), 2.65 (s, 1.5 H), 2.61 (m, 1.5 H), 2.02 (m, 1 H), 1.87-1.34 (m, 10 H), 1.02 (t, J=7.0 Hz, 3 H); MS: ESI(+) 331.2 (M+H⁺, rel. abund 100). Anal. calcd for C₁₆H₂₂N₆O₂: C, 58.17; H, 6.71; N, 25.44. Found: C, 58.01; H, 6.79; N, 25.30.

### Example 24

The compounds listed in Table 4 were prepared using the procedures outlined in Examples 17-23. Compounds were tested in the CMV assay described above and exhibited the following levels of activity: +, IC₅₀>500 nM; ++, 100 nM<IC₅₀<500 nM; +++, IC₅₀<100 nM.

TABLE 4

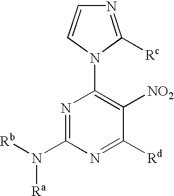

| | | | | m/z (m + 1) | |
|---|---|---|---|---|---|
| R^{a} | R^{b} | R^{c} | R^{d} | or mp (° C.) | IC₅₀ |
| 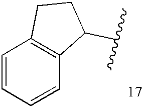 | H | Me | Me | 351.2 | +++ |
| 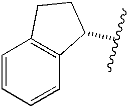 | H | Me | Me | 351.2 | +++ |
| 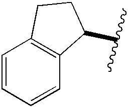 | H | Me | Me | 351.2 | +++ |
| 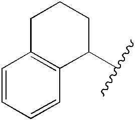 | H | Me | Me | 365.1 | ++ |
| 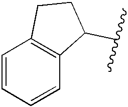 | Me | Me | Me | 365.1 | ++ |
| 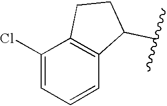 | H | Me | Me | 385.1 | + |
| 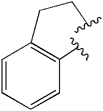 | | Me | Me | 181-182 | ++ |
| 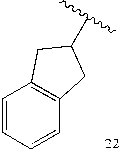 | H | Me | Me | 203-204 | + |
| 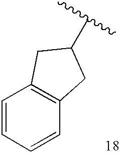 | H | H | Me | 177-178 | + |
| 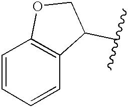 | H | Me | Me | 353.1 | ++ |
| 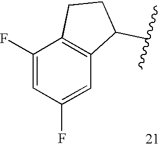 | H | Et | Me | 88-89 | ++ |
| 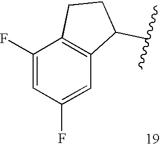 | H | H | Me | 175-176 | ++ |
| 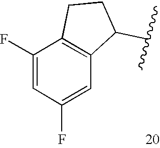 | H | Me | Me | 164-165 | ++ |
| 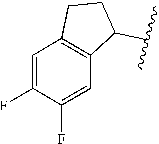 | H | H | Me | 189-190 | ++ |
| 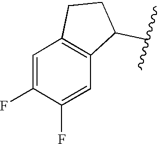 | H | Et | Me | 177-178 | ++ |
| 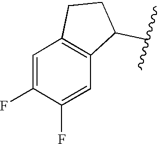 | H | Me | Me | 205-206 | ++ |
| 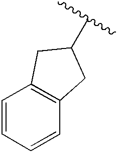 | H | Et | Me | 187-188 | + |
| 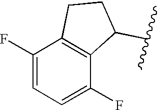 | H | H | Me | 153-154 | ++ |
| 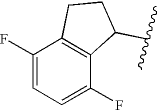 | H | Me | Me | 140-141 | +++ |
| 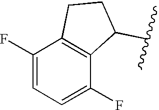 | H | Et | Me | 158-159 | +++ |
| 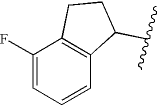 | H | H | Me | 178-179 | ++ |
| 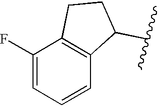 | H | Me | Me | 74-75 | ++ |
| 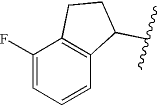 | H | Et | Me | 65-66 | ++ |
| 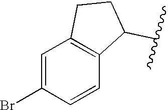 | H | Me | Me | 429.1 | ++ |
| 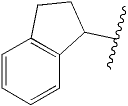 | H | H | Me | 337.1 | +++ |
| 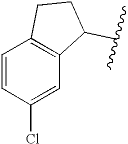 | H | Me | Me | 385.1 | ++ |
| 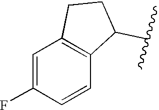 | H | H | Me | 355.1 | +++ |
| 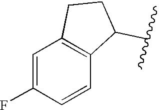 | H | Me | Me | 369.2 | ++ |
| 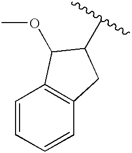 | H | H | Me | 367.3 | + |
| 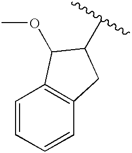 | H | Me | Me | 381.2 | + |
| 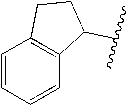 | H | Et | Me | 365.1 | ++ |
| 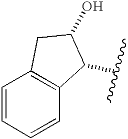 | H | Me | Me | 367.3 | +++ |
| 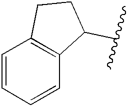 | H | Me | H | 337.1 | ++ |
| 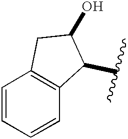 | H | H | Me | 353.1 | ++ |
| 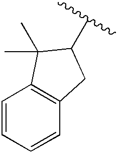 | H | H | Me | 365.1 | + |
| 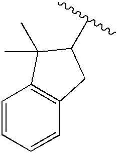 | H | Me | Me | 379.2 | ++ |
| 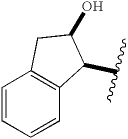 | H | Me | Me | 367.2 | |
| 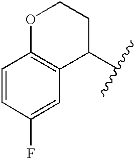 | H | H | Me | 371.1 | |
| 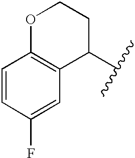 | H | Me | Me | 385.2 | |

### Example 25

The compounds provided in this example were prepared using procedures outlined above. The starting materials are available as described above, or from commercial sources.

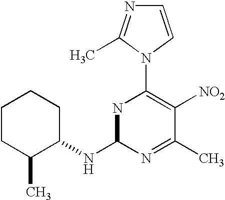

2-(N-2-trans-methylcyclohexylamino)-4-(2-methylimidazolyl)-6-methyl-5-nitropyrimidine (25.1). 125 mg. ¹H NMR (400 MHz, CDCl₃): δ0.92(1.5H, d, J=7.2 Hz); 0.94(1.5H, d, J=7.2 Hz); 1.00-1.30(5H, m); 1.31-1.41(1H, m); 1.74-1.82(2H, m); 1.94-1.96(1H, m); 2.39(1.5H, s); 2.47(1.5H, s); 2.48(1.5H, s); 2.53(1.5H, s); 3.52(0.5H, dq, J=4.0, 9.8 Hz); 3.69(0.5H, dq, J=4.0, 9.8 Hz); 5.86(0.5H, d, J=9.2 Hz), 5.98(0.5H, d, J=9.2 Hz); 6.86(1H, s); 6.93(0.5H, s); 6.95(0.5H, s). MS SEI m/z relative intensity:M+H, 331.2(100)

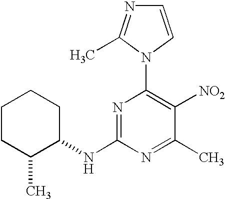

2-(N-2-cis-methylcyclohexylamino)-4-(2-methylimidazolyl)-6-methyl-5-nitropyrimidine (25.2). 85 mg. ¹H NMR (400 MHz, CDCl₃): δ0.93(3H, d, J=7.2 Hz); 1.22-1.41(3H, m); 1.48-1.68 (4H, m); 1.71-1.78(1H, m); 1.95(1H, m); 2.44(1.5H, s); 2.51(3H, s); 2.57(1.5H, s); 4.13(0.5H, m); 4.28(0.5H, m); 5.68(0.5H, d, J=9.0 Hz), 5.59(0.5H, d, J=9.0 Hz); 6.87(1H, s); 6.94(0.5H, s); 6.96(0.5H, s). MS SEI m/z relative intensity:M+H, 331.2(100)

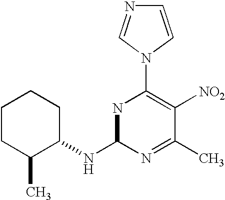

2-(N-2-trans-methylcyclohexylamino)-4-imidazolyl-6-methyl-5-nitropyrimidine (25.3). 48 mg. ¹H NMR (400 MHz, CDCl₃): δ0.96(3H, d, J=6.5 Hz); 1.11-1.29(3H, m); 1.33-1.39(2H, m); 1.70(1H, m); 1.75-1.83(2H, m) 2.05(1H, dd, J=2.8, 13.4 Hz); 2.45(1.5H, s); 2.50(1.5H, s); 3.54(0.5H, dq, J=4.0, 9.8 Hz); 3.70(0.5H, dq, J=4.0, 9.8 Hz); 5.43(0.5H, s), 5.46(0.5H, s); 7.12(0.5H, s); 7.15(0.5H, s); 7.17(0.5H, s); 7.18(0.5H, s); 8.04 (0.5H, s); 8.08(0.5H, s). MS SEI m/z relative intensity:M+H, 317.2(100)

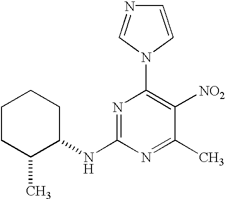

2-N-2-cis-methylcyclohexylamino)-4-imidazolyl-6-methyl-5-nitropyrimidine (25.4). 62 mg. ¹H NMR (400 MHz, CDCl₃): δ0.93(3H, d, J=7.2 Hz); 1.22-1.41(3H, m); 1.48-1.68 (4H, m); 1.76-1.82(1H, m); 1.94-1.99(1H, m); 2.48(1.5H, s); 2.52(1.5H, s); 4.15(0.5H, m); 4.29(0.5H, m); 5.65(0.5H, d, J=7.6 Hz), 5.73(0.5H, d, J=7.6 Hz); 7.16(1H, s); 7.21(1H, s); 8.04(0.5H, s); 8.10(0.5H, s). MS SEI m/z relative intensity:M+H, 317.2(100)

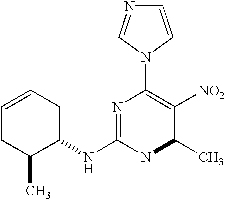

2-(N-2-trans-methyl-4-cyclohexenylamino)-4-imidazolyl-6-methyl-5-nitropyrimidine (25.5). 48 mg. ¹H NMR (400 MHz, CDCl₃): δ0.93(1.5H, d, J=6.8 Hz); 1.00(1.5H, d, J=6.8 Hz); 1.22(1H, m); 1.83-1.88(1H, m); 1.93-2.00(1H, m); 2.12(1H, m) 2.27(1H, m); 2.44(1.5H, s); 2.49(1.5H, s); 3.93(0.5H, dq, J=1.2, 7.2 Hz); 4.08(0.5H, dq J=1.2, 7.2 Hz); 5.51(0.5H, d, J=7.0 Hz), 5.60(1.5H, m); 5.68(0.5H, m); 7.13(1H, s); 7.16(1H, s); 8.00(0.5H, s); 8.07(0.5H, s). MS SEI m/z relative intensity:M+H, 315.2(100)

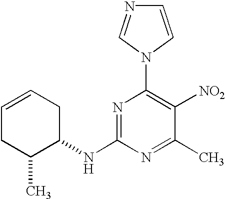

2-(N-2-cis-methyl-4-cyclohexenylamino)-4-imidazolyl-6-methyl-5-nitropyrimidine (25.6). 56 mg. ¹H NMR (400 MHz, CDCl₃): 5 0.96(3H, d, J=6.8 Hz); 1.26(1H, m); 1.84-1.92(1H, m); 2.10-2.18(1H, m); 2.27(1H, m) 2.42(1H, m); 2.47(1.5H, s); 2.51(1.5H, s); 4.32(0.5H, m); 4.47(0.5H, m); 5.63(1H, s), 5.72(1H, s); 5.79(0.5H, d, J=9.0 Hz); 5.88(0.5H, d, J=9.0 Hz); 7.13(0.5H, s); 7.15(0.5H, s); 7.17(0.5H,s); 7.21(0.5H, s); 8.03(0.5H, s); 8.08(0.5H, s). MS SEI m/z relative intensity:M+H, 315.2(100)

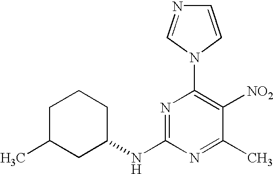

2-(N-3-trans-methylcyclohexylamino)-4-imidazolyl-6-methyl-5-nitropyrimidine (25.7). 206 mg. ¹H NMR (400 MHz, CDCl₃): δ0.93(1.5H, d, J=6.5 Hz); 0.96(0.5H, d, J=6.5 Hz); 1.01-1.12(1H, m); 1.33-1.41(1H, m); 1.45-1.54(1H, m); 1.60-1.83(5H, m); 2.40(1.5H, s); 2.49(1.5H, s); 2.50(1.5H, s); 2.56(1.5H, s); 4.19(0.5H, m); 4.32(0.5H, m); 5.98(0.5H, d, J=6.0 Hz), 6.03(0.5H, d, J=6.0 Hz); 6.88(1H, s); 6.96(1H, s). MS SEI m/z relative intensity:M+H, 331.2(100)

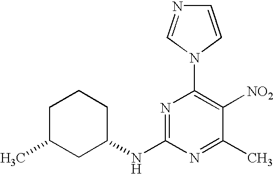

2-(N-3-cis-methylcyclohexylamino)-4-imidazolyl-6-methyl-5-nitropyrimidine (25.8). 62 mg. ¹H NMR (400 MHz, CDCl₃): δ0.90(3H, d, J=6.5 Hz); 1.08(1H, m); 1.29-1.38(1H, m); 1.42-1.52(1H, m); 1.60-1.70(1H, m); 1.76(1H, m); 1.92-2.03(4H, m); 2.36(1.5H, s); 2.46(1.5H, s); 2.49(1.5H, s); 2.54(1.5H, s); 3.73(0.5H, m); 3.91(0.5H, m); 6.06(0.5H, bs), 6.22(0.5H, bs); 6.85(1H, s); 6.93(1H, s). MS SEI m/z relative intensity:M+H, 331.2(100)

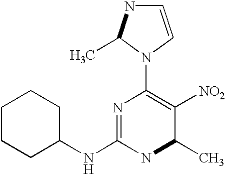

2-cyclohexylamino-4-(2-methylimidazolyl)-6-methyl-5-nitropyrimidine (25.9). 43 mg. ¹H NMR (400 MHz, CDCl₃): δ1.39(2H, m); 1.53(2H, m); 1.74(2H, m); 1.90(2H, m); 2.15(2H, m); 2.58(1.5H, s); 2.65(1.5H, s); 2.67(1.5H, s); 2.72(1.5H, s); 3.95(0.5H, m); 4.10(0.5H, m); 5.68(0.5H, d, J=4.0 Hz), 5.79(0.5H, d, J=4.0 Hz); 7.03(1H, s); 7.12(1H, s). MS SEI m/z relative intensity:M+H, 317.2(100)

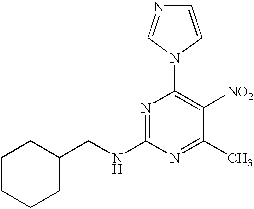

2-cyclohexylmethyamino-4-imidazolyl-6-methyl-5-nitropyrimidine (25.10). 43 mg. ¹H NMR (400 MHz, CDCl₃): δ0.93-1.03(2H, m); 1.12-1.28(3H, m); 1.50-1.61(1H, m); 1.53-1.80(5H, m); 2.44(1.5H, s); 2.50(1.5H, s); 3.31(2H, dt, J=6.5, 24 Hz); 5.88(0.5H, bs); 6.40(0.5H, bs); 7.10(0.5H, s); 7.13(1.5H, s), 7.19(0.5H, s); 8.07(1H, s). MS SEI m/z relative intensity:M+H, 317.2(100)

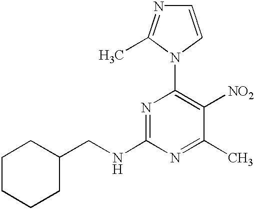

2-(cyclohexylmethyl)amino-4-(2-methylimidazolyl)-6-methyl-5-nitropyrimidine (25.11). 43 mg. ¹H NMR (400 Hz, CDCl₃): δ0.96(2H, m); 1.14-1.30(4H, m); 1.55(1H, m); 1.67(1H, m); 1.67-1.80(5H, m); 2.39(1.5H, s); 2.47(1.5H, s); 2.49(1.5H, s); 2.54(1.5H, s); 3.25(0.5H, t, J=6.3 Hz); 3.35(0.5H, t, J=6.3 Hz); 6.02(1H, bs), 6.86(1H, s); 6.95(1H, s). MS SEI m/z relative intensity:M+H, 331.2(100)

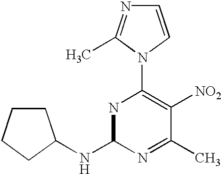

2-(cyclopentylamino-4-(2-methylimidazolyl)-6-methyl-5-nitropyrimidine (25.12). 25 mg. ¹H NMR (400 MHz, CDCl₃): δ1.21(1H, m); 1.49(1H, m); 1.60-1.78(4H, m); 2.38(1.5H, s); 2.47(1.5H, s); 2.55(1.5H, s); 4.21(0.5H, m); 4.37(0.5H, m); 5.86(0.5H, d, J=4.2 Hz); 5.98(0.5H, d, J=4.2 Hz); 6.86(1H, s); 6.95(1H, s). MS SEI m/z relative intensity:M+H, 303.2(100)

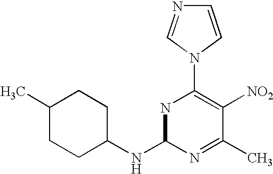

2-(N-(4-methylcyclohexyl)amino)-4-imidazolyl-6-methyl-5-nitropyrimidine (25.13). 28 mg. ¹H NMR (400 MHz, CDCl₃): δ1.03(1.5H, d, J=6.2 Hz); 1.06(1.5H, d, J=6.2 Hz); 1.08(1H, m); 1.15-1.28(1H, m); 1.30-1.42(2H, m); 1.43-1.55(1H, m); 1.70-1.84(4H, m); 1.85-1.96(2H, m); 2.18(1H, m); 2.54(1.5H, s); 2.64(3H, s); 2.69(1.5H, s); 3.84(0.5H, m); 4.02(0.5H, m); 5.97(0.5H, bs), 6.11(0.5H, bs); 7.01(1H, s); 77.10(11H, s). MS SEI m/z relative intensity:M+H, 331.1(100)

### Example 26

This example illustrates the synthesis of two salts of Compound 1, according to the route shown in FIG. 16.

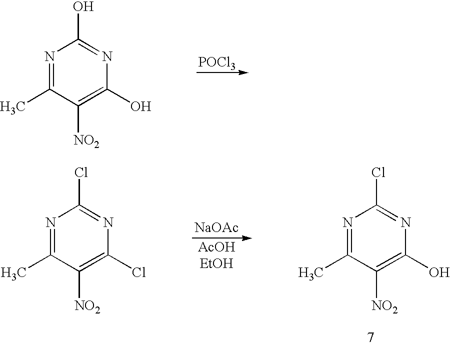

26.1 2-chloro-4-hydroxy-6-methyl-5-nitropyrimidine (7). A 5 L flask was charged with tetraethylammonium chloride (590 g) which was then heated at 60° C. under vacuum for 22 h to remove any water. The flask was then charged with 3 L anhydrous CH₃CN, 2,4-dihydroxy-6-methyl-5-nitropyrimidine (295 g, 1.724 mol, 1.0 equiv.), dimethylaniline (221 mL, 2.98 mol, 1.74 equiv.), and 1 L POCl₃ (18.5 mol, 10.73 equiv.). The flask was equipped with a condenser and the temperature was increased to 80° C. under N₂. After stirring for 29 h the hot black solution was poured onto 14 L ice and allowed to stir for 30 min while a yellow precipitate formed. The suspension was filtered, and the solid was washed 3×1.0 N HCl to give 247.3 g of dichloropyrimidine product. The aqueous solution from the filtration was extracted (3×CH₂Cl₂), dried (Na₂SO₄) and concentrated under reduced pressure. The resulting green oil was purified via flash chromatography (SiO₂, 1:1 hexanes:CH₂Cl₂), and the resulting light green solid was crystallized from hot hexanes. The crystals were washed with hexanes to give an additional 63.58 g of the yellow product. The combined yield of the dichloropyrimidine product was 310.88 g (1.495 mol, 87%).

The dichloropyrimidine prepared in the manner described above (150.0 g, 721 mmol) was dissolved in 3 L EtOH and cooled to 0° C. In a separate flask were combined 910 mL H₂O, 922 mL AcOH, and 90 g NaOAc. The aqueous solution was then added dropwise to the dichloride solution via dropping funnel over a period of 2 h. The solution was allowed to stir for 24 h by which time a light yellow precipitate was formed. The solid product was filtered off and the aqueous solution was set aside. The solid product was washed (3×200 mL EtOH) to give 70.3 g of the product as a fluffy light yellow solid. The aqueous solution was recooled to 0° C. followed by the addition of an additional 140 g dichloropyrimidine (673 mmol) and 84.5 g NaOAc. The resulting slurry was allowed to stir a further 24 h, at which time an additional 120.87 g of product was obtained via filtration as above. The remaining aqueous solution was allowed to stir at 0° C. for an additional 24 h, followed by filtration as above to give an additional 24.03 g product. Total product recovered was 215.2 g (1.135 mol, 79%) as a white solid: mp 242-244° C. (dec); IR (KBr) 3349, 1657, 1600, 1507, 1419, 1352, 1276, 1188, 1100, 998, 945, 799, 696, 624 cm−1; ¹H NMR (CDCl₃, 400 MHz) δ1.53 (s, 3 H); ESI-MS m/z 212.0 (M+Na⁺).

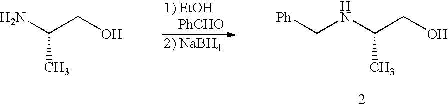

26.2 N-Benzyl-L-alaninol (2). (S)-2-Aminopropanol (300 g, 3.994 mol, 1.0 equiv.) was dissolved in 3.0 L of anhydrous EtOH in a 5 L three neck flask under N₂. Benzaldehyde (406.05 mL, 3.994 mol, 1.0 equiv.) was added in one portion, and the slightly warm solution was allowed to stir for 2.5 h. The solution was then cooled to 0° C. in an ice bath, followed by the addition of 196.5 g NaBH₄ (5.194 mol, 1.3 equiv.) over a period of 20 min. After stirring for 20 h 521 mL H₂O was added via addition funnel over a period of 60 min. The resulting white slurry was then diluted with 3.0 L CH₂Cl₂ and stirred for an additional 5 h. The slurry was then filtered, and the solids were washed with three portions of CH₂Cl₂. The clear solution obtained from the filtration was then concentrated under reduced pressure to a volume of ˜800 mL. The solution was then diluted with 2 L H₂O, extracted (3×1.4 L CH₂Cl₂), dried (Na₂SO₄), and concentrated under reduced pressure to give a thick colorless oil, which quickly crystallized upon standing. The white solid was triturated with 1.0 L hexanes, filtered, and washed with hexanes (3×500 mL) to give the pure product as a white solid 648.65 g (3.926 mol, 98%): mp 39-40° C.; [α]²⁵_{D=+}38.5° (c=1.04, MeOH); IR (KBr) 3293, 3060, 3024, 2957, 2911, 2844, 1495, 1453, 1380, 1347, 1149, 1061, 965, 935, 873, 779, 746, 699, 611 cm⁻¹; ¹H NMR (CDCl₃, 400 MHz) δ7.32 (m, 3 H), 7.26 (m, 2 H), 3.88 (d, J=12.8 Hz, 1 H), 3.75 (d, J=12.8 Hz, 1 H), 3.61 (dd, J=4.0, 10.6 Hz, 1 H), 3.28 (dd, J=7.0, 10.6 Hz, 1 H), 2.86 (ddddd, J=4.0, 6.6×3, 6.9 Hz, 1 H), 1.78 (broad singlet, 2 H), 1.10 (d, J=6.2 Hz, 3 H); ESI-MS m/z 166.2 (100, M+H⁺). Anal. Calcd for C₁₀H₁₅NO: C, 72.68; H, 9.15; N, 8.48. Found: C, 72.85; H, 9.06; N, 8.55.

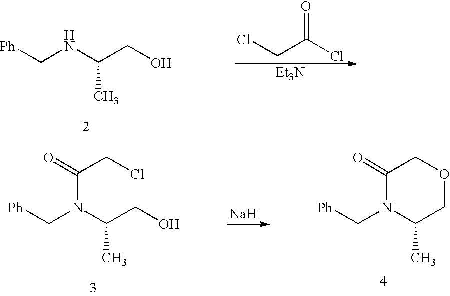

26.3 5S-N-Benzylmorpholin-3-one (4). A 12 L three neck flask equipped with mechanical stirrer was charged with 648.65 g of N-benzyl-L-alaninol (3.926 mol, 1.0 equiv.) and 4.0 L CH₂Cl₂. The solution was cooled to −10° C. in a methanol-ice bath followed by the addition of 547 mL Et₃N (3.926 mol, 1.0 equiv.). Chloroacetyl chloride (312.2 mL, 3.926 mol, 1.0 equiv.) was dissolved in 700 mL CH₂Cl₂, and the chloride solution was added dropwise via addition funnel resulting in a cloudy tan solution. The solution was stirred for 1 h, and was then diluted with 3 L H₂O. After stirring rapidly for 5 min, the layers were separated, and the water layer was extracted (3×700 mL CH₂Cl₂). The combined organics were washed (1×2 L H₂O), dried (500 g Na₂SO₄), and concentrated under reduced pressure to give amide 3 as a light red viscous oil, which was used directly in the cyclization step.

A 12 L flask equipped with a reflux condenser and mechanical stirrer was charged with 94.22 g NaH (3.92 mol, 1.0 equiv., Aldrich 95%) followed by 3.0 L anhydrous THF. The 2-chloroamide from above was dissolved in 3.0 L anhydrous THEF and transferred via cannula to the NaH solution over a period of 25 min. The slurry was then slowly heated to 65° C. over 60 min. After stirring for 4.5 h at reflux, the sodium hydride was then quenched by the slow addition of 100 mL H₂O in 100 mL THF via dropping funnel. The heating mantle was then removed, and the reaction was allowed to cool down with stirring overnight. The majority of the THF was removed under reduced pressure, and the resulting slurry was diluted with 3 L CH₂Cl₂. The solid salts were filtered off, washed (3×CH₂Cl₂), and discarded. The resulting clear solution was diluted with 3 L H₂O, and extracted (5×700 mL CHCl₃). The combined organics were dried (Na₂SO₄), and concentrated under reduced pressure. Purification by flash chromatography (SiO₂, 100% CH₂Cl₂ to 5% MeOH/CH₂Cl₂) gave the product as a colorless oil 594.92 g (2.90 mol, 74%). [α]²⁵_{D}=−90° (c=1.0, MeOH); ¹H NMR (CDCl₃, 400 MHz) δ7.36-7.24 (m, 5 H), 5.38 (d, J=15.4 Hz, 1 H), 4.29 (d, J=16.5 Hz, 1 H), 4.23 (d, J=16.8 Hz, 1 H), 3.97 (d, J=15.0 Hz, 1 H), 3.75 (dd, J=3.3, 11.7 Hz, 1 H), 3.64 (dd, J=3.3, 11.7 Hz, 1 H), 3.56 (m, 1 H), 1.28 (d, J=6.2 Hz, 3 H); ESI-MS m/z 206.1 (100, M+H⁺), 228.2 (45, M+Na⁺). Anal. Calcd for C₁₂H₁₅NO₂: C, 70.22; H, 7.37; N, 6.82. Found: C, 70.16; H, 7.39; N, 6.83.

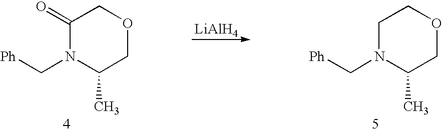

26.4 3S-N-Benzyl-3-methylmorpholine (5). A 12 L three neck flask equipped with mechanical stirring device, heating mantle, and reflux condenser was charged with 220.14 g 95% LiAlH₄ (5.80 mol, 2.0 equiv.) followed by 5.2 L anhydrous THF under N₂. The gray slurry was allowed to stir for 30 min. The N-Benzylmorpholin-3-one 4 (594.92 g, 2.90 mol, 1.0 equiv.) was dissolved in 2 L THF, and added via addition funnel over the course of 3.5 h. The solution was then heated to reflux and allowed to stir at reflux for 19.5 h. The solution was then cooled to rt, followed by the careful addition of 220 mL H₂O in 665 mL THF via addition funnel over a period of 14 h. When all evolution of gas was stopped, 220 mL 15% NaOH was added, followed by 660 mL H₂O. The white slurry was then stirred for 56 h. The slurry was filtered through a fritted funnel, and the solids were washed (5×600 mL Et₂O). The clear ethereal solution was then concentrated under reduced pressure to give the product as a colorless oil 509.04 g (2.66 mol, 92%). [α]²⁵_{D}=+94.5° (c=1.10, MeOH);¹H NMR (CDCl₃, 400 MHz) δ7.22-7.35 (m, 5 H), 4.06 (d, J=13.2 Hz, 1 H), 3.72 (m, 2 H), 3.59 (ddd, J=2.6, 10.3, 11.3 Hz, 1 H), 3.31 (dd, J=9.2, 11.3 Hz, 1 H), 3.14 (d, J=13.5 Hz, 1 H), 2.59 (ddd, J=2.6, 2.9, 12.1 Hz, 1 H), 2.49 (m, 1 H), 2.19 (ddd, J=3.3, 9.9, 12.1 Hz, 1 H), 1.09 (d, J=6.2 Hz, 3 H); ESI-MS m/z 192.2 (100, M+H⁺). Anal. Calcd for C₁₂H₁₇NO: C, 75.35; H, 8.96; N, 7.32. Found: C, 75.48; H, 8.96; N, 7.23.

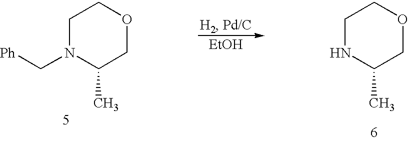

26.5 3S-3-Methylmorpholine (6). 3S-N-Benzyl-3-methyl-morpholine (130.0 g, 680 mmol, 1.0 equiv.) was dissolved in 200 mL EtOH and transferred to a Parr vessel. 10.0 g of Pd/C (10 wt % Pd) was added, and the Parr flask was sealed and subjected to hydrogenation on a Parr shaker at 62 PSI. Hydrogen pressure was adjusted periodically throughout the hydrogenation to maintain 60 PSI. After 44 h, the hydrogenation was stopped and the vessel was purged with nitrogen. The solution was filtered through a plug of Celite, and the ethanolic solution was used directly in the next step.

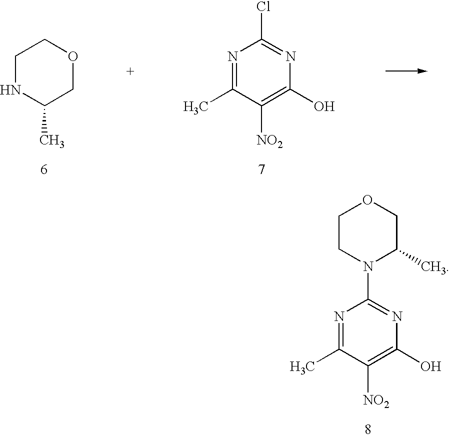

26.6 2-(3S-3-methylmorpholino)-4-hydroxy-6-methyl-5-nitropyrimidine (8). To the ethanolic solution of 3S-3-methylmorpholine 6 prepared above (˜680 mmol, 3.2 equiv.) in a 1 L flask under N₂ was added 2-chloro-4-hydroxy-6-methyl-5-nitropyrimidine (40.0 g, 211 mmol, 1.0 equiv.) and 17.30 g anhydrous NaOAc (211 mmol, 1.0 equiv.). The flask containing the light yellow slurry was equipped with a condenser and placed into a preheated oil bath at 80° C. After 24 h an additional 17.3 g NaOAc (211 mmol, 1.0 equiv.) and 35.0 g potassium iodide (211 mmol, 1.0 equiv.) was added to the bright orange slurry. After heating for an additional 21 h the flask was removed and the solution was allowed to cool to rt. The suspension was then filtered, and the solids were washed (3×50 mL EtOH). The combined ethanol solution was then concentrated to ˜100 mL under reduced pressure and diluted with 0.5 N HCl until the pH was ˜2. The solution was extracted (3×500 mL CH₂Cl₂), washed with 1.0 N HCl, dried (Na₂SO₄), and concentrated under reduced pressure to give the crude yellow solid. Purification via flash chromatography (SiO₂, 2-4% MeOH/CH₂Cl₂) gave the product as a yellow solid 40.86 g (160.8 mmol, 76%): mp 179-180° C.; [α]²⁵_{D}=+135.1° (c=1.04, MeOH); IR (KBr) 3439, 3121, 2976, 2861, 1669, 1577, 1506, 1389, 1336, 1263, 1136, 1067, 982, 915, 846, 796 cm−1; ¹H NMR (CDCl₃, 400 MHz) δ7.37 (d, J=3.7 Hz, 1 H), 4.72 (m, 1 H), 4.45 (m, 1 H), 4.02 (dd, J=3.7, 11.4 Hz, 1 H), 3.80 (d, J=12.1 Hz, 1 H), 3.67 (dd, J=2.9, 11.7 Hz, 1 H), 3.53 (ddd, J=2.9, 11.7, 12.1 Hz, 1 H), 3.36 (ddd, J=3.7, 12.8, 13.5 Hz, 1 H), 2.58 (s, 3 H), 1.59 (d, J=7.0 Hz, 3 H); ESI-MS m/z 255.1 (100, M+H+). Anal. Calcd for C₁₀H₁₄N₄O₄: C, 47.24; H, 5.55; N, 22.04. Found: C, 47.14; H, 5.48; N, 22.15.

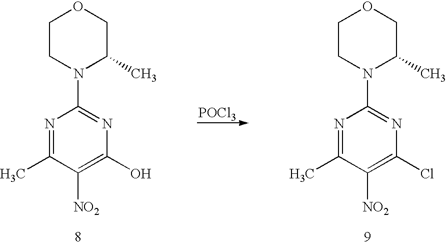

26.7 2-(3S-3-methylmorpholino)-4-chloro-6-methyl-5-nitropyrimidine (9). A 1 L flask containing the nitropyrimidine 8 prepared above (39.99 g, 157.4 mmol, 1.0 equiv.) was charged with 250 mL POCl₃ under N₂. The flask was equipped with a condenser and placed in a preheated 80° C. bath with stirring. The slurry slowly dissolved over a period of 50 min, and the yellow solution was then removed from the bath, and POCl₃ was removed under reduced pressure in a rotary evaporator with a bath temperature of 60° C. The resulting yellow oil was purified via flash chromatography (SiO₂, 10 to 50% EtOAc/Hexanes) to give 40.24 g of the product (147.9 mmol, 94%) as a yellow oil. [α]²⁵_{D}=+144.6° (c=1.03, MeOH); ¹H NMR (CDCl₃, 400 MHz) δ5 4.76 (m, 1 H), 4.44 (m, 1 H), 3.99 (dd, J=4.0, 11.7 Hz, 1 H), 3.78 (d, J=11.7 Hz, 1 H), 3.65 (dd, J=3.3, 11.7 Hz, 1 H), 3.51 (ddd, J=2.9, 11.7, 12.4 Hz, 1 H), 3.32 (ddd, J=4.0, 12.4, 13.9 Hz, 1 H), 2.45 (s, 3 H), 1.34 (d, J=7.0 Hz, 3 H); ESI-MS m/z 273.0 (100, M+H+).

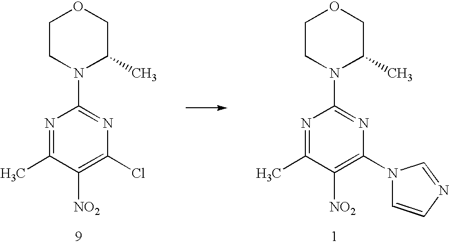

26.8 2-(3S-3-methylmorpholino)-4-(imidazol-1-yl)-6-methyl-5-nitropyrimidine (1). The chloropyrimidine 9 prepared above (40.04 g, 147.2 mmol, 1.0 equiv.) was dissolved in 300 mL anhydrous EtOH followed by the addition of 30.07 g imidazole (441.6 mmol, 3.0 equiv.) under N₂. The flask was equipped with a condenser and placed in a preheated 80° C. bath with magnetic stirring. After stirring for 75 min the solution was cooled to rt and concentrated under reduced pressure. Purification via flash chromatography (SiO₂, 2-4% MeOH/CH₂Cl₂) gave the product as a yellow oil. Upon standing the oil crystallized to give a yellow solid which was triturated with hexanes, filtered, and washed (3×hexanes) to give 40.53 g (133.3 mmol, 91%) of the product as yellow crystals: mp=74-75° C.; [α]²⁵_{D}=+152.6° (c=1.03, MeOH); IR (KBr) 3116, 2972, 2855, 1586, 1482, 1443, 1329, 1315, 1239, 1205, 1129, 1074, 1007, 897, 844, 773, 739, 650 cm−1; ¹H NMR (CDCl₃, 400 MHz) δ8.10 (s, 1 H), 7.21 (m, 1 H), 7.17 (m, 1 H), 4.80 (m, 1 H), 4.48 (m, 1 H), 4.02 (dd, J=3.7, 11.7 Hz, 1 H), 3.80 (d, J=11.7 Hz, 1 H), 3.68(dd, J=3.3, 11.7 Hz, 1 H), 3.52 (ddd, J=2.9, 11.7, 12.1 Hz, 1 H), 3.36 (ddd, J=3.7, 12.9, 13.5 Hz, 1 H), 2.53 (s, 3 H), 1.38 (d, J=7.0 Hz, 3 H); ESI-MS m/z 305.1 (100, M+H+). Anal. Found for C₁₃H₁₆N₆O₃: C, 51.31; H, 5.30; N, 27.62. Found: C, 51.47; H, 5.30; N, 27.79.

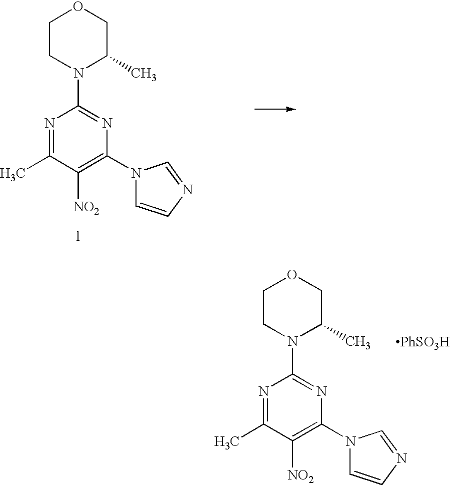

26.9 Compound 1.PhSO₃ salt, (2-(3S-3-methylmorpholino)-4-(imidazol-1-yl)-6-methyl-5-nitropyrimidine benzenesulfonate). A 250 mL flask was charged with 1 (39.88 g, 131.1 mmol, 1.0 equiv.) and 100 mL EtOH under N₂. The suspension was heated to 50° C. until everything dissolved. Benzenesulfonic acid hydrate (20.81 g, 131.1 mmol, 1.0 equiv.) was added via spatula, and additional EtOH was used to wash the all of the solids into the flask (5 mL). Hexane (20 mL) was added to the solution, which was then stirred rapidly for 5 min. Crystals began to form within the first 5 min after stirring was stopped, and the flask was allowed to cool to rt overnight. The crystals which formed overnight were filtered and washed (5×50 mL EtOH) to give the product besylate salt 51.988 g (112.4 mmol, 86%): Yellow crystals mp 184.5° C.; [α]²⁵_{D}=+115.6° (c=1.00, MeOH); IR (KBr) 3442, 3129, 2985, 2862, 1597, 1546, 1529, 1490, 1443, 1317, 1231, 1182, 1123, 1072, 1014, 892, 846, 786, 727, 612,564 cm−1; ¹H NMR (400 MHz, CD₃OD) δ9.52 (s, 1 H), 7.93 (s, 1 H), 7.81 (m, 2 H), 7.42 (s, 1 H), 7.44-7.37 (m, 3 H), 4.94 (m, 0.5 H), 4.74 (m, 0.5 H), 4.62 (m, 0.5 H), 4.62 (m, 0.5 H), 4.01 (m, 1 H), 3.79 (m, 1 H), 3.67 (dd, J=3.3, 11.8 Hz, 1 H), 3.53 (ddd, J=2.8, 11.3, 12.3 Hz, 1 H), 3.42 (ddd, J=3.6, 12.8, 13.2 Hz, 1 H), 2.68 (s, 3 H), 1.36 (d, J=6.9 Hz, 3 H). Anal. Calcd for C₃H₁₆N₆O₃.C₆H₆O₃S: C, 49.34; H, 4.79; N, 18.17; S, 6.92. Found: C, 49.30; H, 4.75; N, 18.22; S, 6.97.

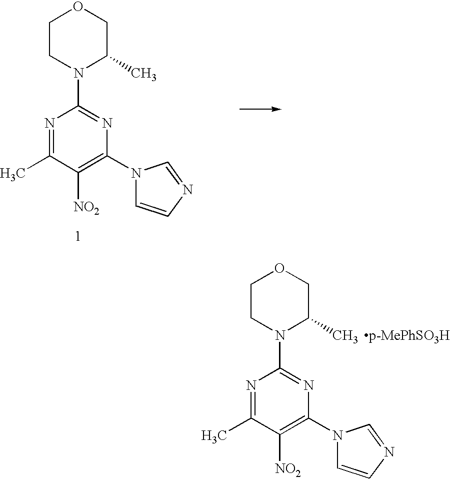

26.10 Compound 1.p-MePhSO₃ salt, (2-(3S-3-methylmorpholino)-4-(imidazol-1-yl)-6-methyl-5-nitropyrimidine p-toluenesulfonate). A 25 mL flask was charged with 822 mg 1 (2.70 mmol, 1.0 equiv.) and 3 mL CH₂Cl₂, followed by the addition of 514 mg (2.70 mmol, 1.0 equiv.) p-toluenesulfonic acid mono hydrate. 1.5 mL hexanes was added and the clear solution was allowed to sit overnight. The solution was then concentrated under reduced pressure and taken up in 3 mL EtOAc whereupon a yellow solid precipitated out. The yellow solid was filtered, and washed (3×1:1 hexanes:EtOAc) to give 1.202 g. The yellow solid was recrystallized from 5:1 CHCl₃:hexanes to give 1.078 g of product (2.26 mmol, 84%) salt after filtration and washing (2×1:1 CHCl₃:hexanes): mp 168-169° C.; [α]²⁵_{D}=+104.9° (c=1.05, MeOH); IR (KBr) 3128, 2981, 2858, 1595, 1544, 1526, 1442, 1317, 1227, 1184, 1123, 1030, 1007, 683, 562 cm−1; ¹H NMR (400 MHz, CD₃OD) δ9.53 (s, 1 H), 7.94 (m, 1 H), 7.74 (m, 1 H), 7.69 (d, J=8.4 Hz, 2 H), 7.21 (d,=8.4 Hz, 2 H), 4.94 (m, 0.5 H), 4.75 (m, 0.5 H), 4.62 (m, 0.5 H), 4.41 (m, 0.5 H), 3.99 (m, 1 H), 3.80 (m, 1 H), 3.67 (dd, J=2.9, 11.7 Hz, 1 H), 3.53 (ddd, J=2.6, 11.7, 12.1 Hz, 1 H), 3.41 (ddd, J=3.7, 12.5, 13.6 Hz, 1 H), 2.68 (s, 3 H), 2.36 (s, 3 H), 1.37 (d, J=7.0 Hz, 1 H). Anal. Calcd for C₁₃H₁₆N₆O₆.C₇H₈O₃S: C, 50.41; H, 5.08; N, 17.64; S, 6.73. Found: C, 49.88; H, 4.75; N, 18.23; S, 6.91.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating or preventing a disease associated with CMV infection, comprising
administering to a subject in need thereof a therapeutically effective amount of a compound of formula (I): 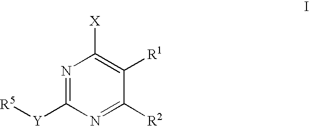
wherein
X is a member selected from the group consisting of —NR³R⁴, —OR³, —SR³, aryl, alkyl and arylalkyl;
Y is a member selected from the group consisting of a covalent bond, —N(R⁶)—, —O—, —S—, —C(═O)— and alkylene;
R¹ and R² are members independently selected from the group consisting of hydrogen, alkyl, —O-alkyl, —S-alkyl, aryl, arylalkyl, —O-aryl, —S-aryl, —NO₂, —NR⁷R⁸, —C(O)R⁹, —CO₂R¹⁰, —C(O)NR⁷R⁸—N(R⁷)C(O)R⁹, —N(R⁷)CO₂R¹¹, —N(R⁹)C(O)NR⁷R⁸, —S(O)ₘNR⁷R⁸, —S(O)ₙR⁹, —CN, halogen, and —N(R⁷)S(O)ₘR¹¹;
R³ and R⁴ are members independently selected from the group consisting of hydrogen, alkyl, aryl and arylalkyl, or combined to form a 5-, 6- or 7-membered ring containing from one to three heteroatoms in the ring;
R⁵ is a member selected from the group consisting alkyl, aryl, arylalkyl and bicyclic fused aryl-cycloalkyl;
R⁶ is a member selected from the group consisting of hydrogen, alkyl, aryl and arylalkyl; or is combined with R⁵ and the nitrogen atom to which R⁵ and R⁶ are attached to form a 5-, 6-, 7- or 8-membered ring;
R⁷ and R⁸ are members independently selected from the group consisting of hydrogen, alkyl, aryl and arylalkyl, or, combined to form a 4-, 5-, 6-, 7- or 8-membered ring containing from one to three heteroatoms in the ring;
R⁹ and R¹⁰ are members independently selected from the group consisting of hydrogen, alkyl, aryl and arylalkyl;
R¹¹ is a member selected from the group consisting of alkyl, aryl and arylalkyl;
m is an integer of from 1 to 2;
n is an integer of from 1 to 3; and
optionally, a 5-, 6-, 7- or 8-member ring is formed by joining R¹ to R², R¹ to R³, R³ to N³, R⁵ to N³, R⁵ to N¹, or R² to N¹;
with the proviso that when Y is a bond, then R⁵ is other than an imidazole ring.

2. The method of claim 1, wherein R¹ is selected from the group consisting of —NO₂, —S(O)ₘNR⁷R⁸, —S(O)ₙR⁹, —CN, fluoroalkyl, —C(O)R⁹, —CO₂R¹⁰ and —C(O)NR⁷R⁸ and R² is selected from the group consisting of hydrogen, alkyl, —O-alkyl, —S-alkyl, aryl, arylalkyl, —O-aryl and —S-aryl.

3. The method of claim 1, wherein X is —NR³R⁴, Y is selected from the group consisting of—N(R⁶)—, —O— and —S—, R¹ is selected from the group consisting of —C(O)R⁹, —C(O)NR⁷R⁸, —S(O)ₙR⁹, —S(O)ₘNR⁷R⁸, —CO₂R¹⁰, —CN, fluoroalkyl and —NO₂, and R² is a member selected from the group consisting of hydrogen, alkyl, —O-alkyl and halogen.

4. The method of claim 1, wherein R¹ is selected from the group consisting of —CF₃, —S(O)ₘNR⁷R⁸, —CO₂R¹⁰, —CN and —NO₂, and R² is selected from the group consisting of hydrogen, (lower)alkyl, —O-(lower)alkyl and —S-(lower)alkyl.

5. The method of claim 1, wherein Y is —N(R⁶)— or —O—, R¹ is —NO₂, and R² is hydrogen or (C₁-C₄)alkyl.

6. The method of claim 1, wherein R³ is joined to R⁴ to form a 5-membered ring, together with the nitrogen to which both radicals are attached.

7. The method of claim 6, wherein said 5-membered ring contains two nitrogen atoms.

8. The method of claim 7, wherein said 5-membered ring is an imidazole ring.

9. The method of claim 1, wherein Y is —N(R⁶)—, in which R⁶ is hydrogen or lower alkyl, and R⁵ is a member selected from the group consisting of alkyl, aryl, arylalkyl and bicyclic fused aryl-cycloalkyl.

10. The method of claim 1, wherein R⁵ is selected from the group consisting of cycloalkyl, heterocycloalkyl, aryl, arylalkyl and bicyclic fused aryl-cycloalkyl, R⁶ is selected from the group consisting of hydrogen, methyl, ethyl and propyl, and —NR³R⁴ is selected from the group consisting of imidazol-1-yl, 2-methylimidazol-1yl, 2-ethylimidazol-1-yl, 2-(1-propyl)imidazol-1-yl and 2-(2-propyl)imidazol-1-yl.

11. The method of claim 1, wherein R⁶ is selected from the group consisting of hydrogen, methyl and ethyl, —NR³R⁴ is selected from the group consisting of imidazol-1-yl, 2-methylimidazol-1yl, 2,4-dimethylimidazol-1-yl and 2-ethylimidazol-1-yl, and R⁵ is an optionally substituted radical selected from the group consisting of

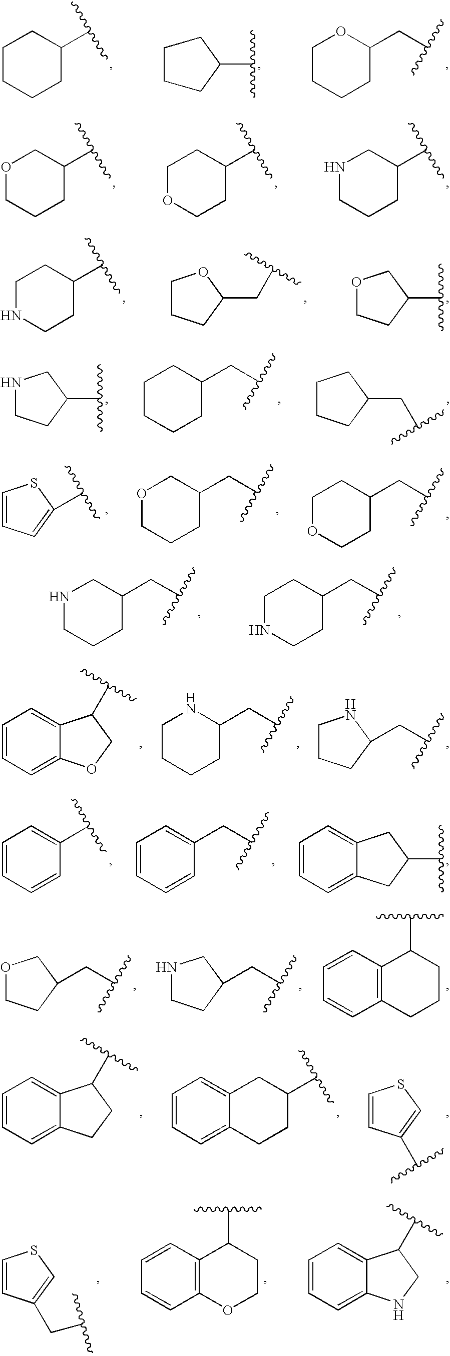

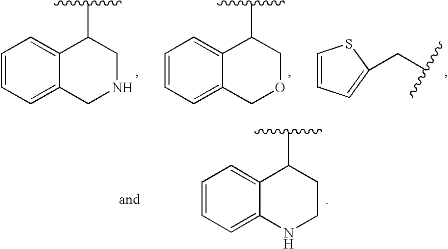

12. The method of claim 1, wherein R⁵ is a member selected from the group consisting of:

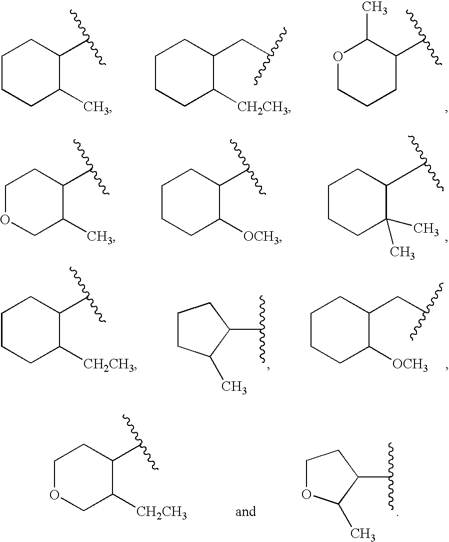

3. The method of claim 1, said compound having the formula: 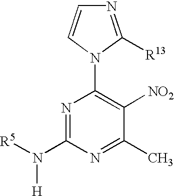
wherein
R¹³ is a member selected from the group consisting of hydrogen, methyl and ethyl; and
R⁵ is a member selected from the group consisting of:

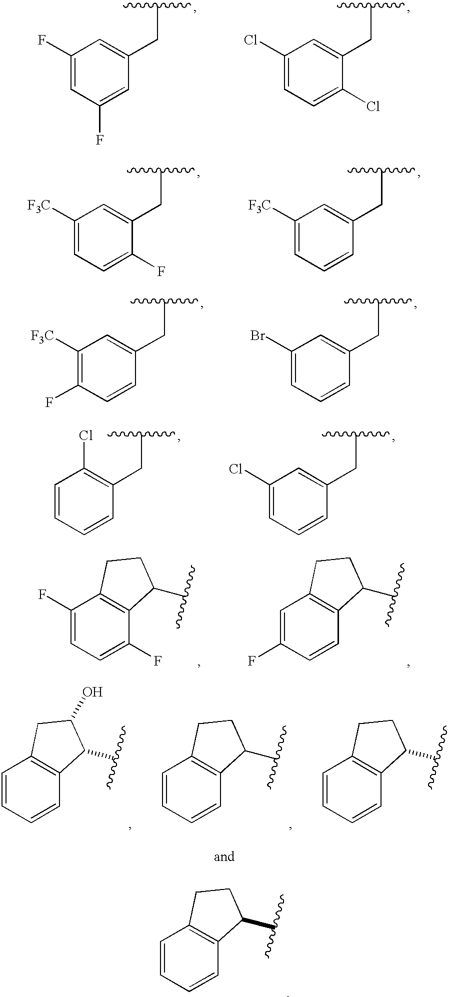

14. The method of claim 13, wherein R¹³ is methyl.

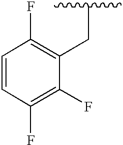

15. The method of claim 1, said compound having the formula: wherein
R¹³ is selected from the group consisting of hydrogen, methyl and ethyl; and
R⁵ and R⁶ are combined with the nitrogen atom to which R⁵ and R⁶ are attached to form a heterocycloalkyl ring.

16. The method of claim 15, said compound having the formula: 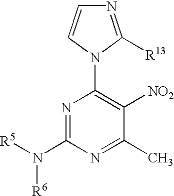
wherein
R¹³ is selected from the group consisting of hydrogen, methyl and ethyl; and
R⁵ and R⁶ are combined with the nitrogen atom to which R⁵ and R⁶ are attached to form a heterocycloalkyl ring.

17. The method of claim 16, wherein said heterocycloalkyl ring is selected from the group consisting of substituted or unsubstituted 1-piperidinyl, substituted or unsubstituted 4-morpholinyl and substituted or unsubstituted 1-pyrrolidinyl.

18. The method of claim 16 wherein R¹³ is hydrogen and R⁵ and R⁶ are combined with the nitrogen atom to which R⁵ and R⁶ are attached to form a substituted or unsubstituted 4-morpholinyl.

19. The method of claim 18, wherein R⁵ and R⁶ are combined with the nitrogen atom to which R⁵ and R⁶ are attached to form a monosubstituted 4-morpholinyl, said substituent being selected from the group consisting of (C₁-C₄)alkyl.

20. The method of claim 19, wherein said compound is selected from the group consisting of

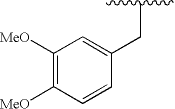

21. The method of claim 1, wherein said disease associated with CMV infection is cardiovascular disease.

22. The method of claim 21 wherein said cardiovascular disease is selected from the group consisting of atherosclerosis and restenosis.

23. The method of claim 21, wherein said compound is administered in combination with a therapeutically effective amount of an agent selected from the group consisting of an antiviral agent, an agent used to treat atherosclerosis and an agent used to treat restenosis.

24. The method of claim 23, wherein said antiviral agent is selected from the group consisting of ganciclovir, valganciclovir, acyclovir, foscarnet, cidofovir and fomivirsen.

25. The method of claim 1, wherein said disease associated with CMV infection is organ transplant rejection or a pathology associated with organ transplantation.

26. The method of claim 25, wherein said organ transplant rejection is selected from the group consisting of allograft rejection and xenograft rejection.

27. The method of claim 25, wherein said compound is administered in combination with an immunosuppressant agent.

28. The method of claim 1, wherein said administering is oral.

29. The method of claim 1, wherein said administering is topical.

30. The method of claim 1, wherein said administering is parenteral.

31. A method of treating a disease selected from the group consisting of cardiovascular disease, organ transplant rejection, organ transplant-associated atherosclerosis and a pathology associated with organ transplantation, comprising
administering to a subject in need thereof a therapeutically effective amount of a compound of formula (I): 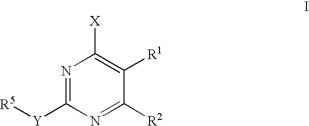
wherein
X is a member selected from the group consisting of —NR³R⁴, —OR³, —SR³, aryl, alkyl and arylalkyl;
Y is a member selected from the group consisting of a covalent bond, —N(R⁶)—, —O—, —S—, —C(═O)— and alkylene;
R¹ and R² are members independently selected from the group consisting of hydrogen, alkyl, —O-alkyl, —S-alkyl, aryl, arylalkyl, —O-aryl, —S-aryl, —NO₂, —NR⁷R⁸, —C(O)R⁹, —CO₂R¹⁰, —C(O)NR⁷R⁸—N(R⁷)C(O)R⁹, —N(R⁷)CO₂R¹¹, —N(R⁹)C(O)NR⁷R⁸, —S(O)ₘNR⁷R⁸, —S(O)ₙR⁹, —CN, halogen, and —N(R⁷)S(O)ₘR¹¹;
R³ and R⁴ are members independently selected from the group consisting of hydrogen, alkyl, aryl and arylalkyl, or combined to form a 5-, 6- or 7-membered ring containing from one to three heteroatoms in the ring;
R⁵ is a member selected from the group consisting alkyl, aryl, arylalkyl and bicyclic fused aryl-cycloalkyl;
R⁶ is a member selected from the group consisting of hydrogen, alkyl, aryl and arylalkyl; or is combined with R⁵ and the nitrogen atom to which R⁵ and R⁶ are attached to form a 5-, 6-, 7- or 8-membered ring;
R⁷ and R⁸ are members independently selected from the group consisting of hydrogen, alkyl, aryl and arylalkyl, or, combined to form a 4-, 5-, 6-, 7- or 8-membered ring containing from one to three heteroatoms in the ring;
R⁹ and R¹⁰ are members independently selected from the group consisting of hydrogen, alkyl, aryl and arylalkyl;
R¹¹ is a member selected from the group consisting of alkyl, aryl and arylalkyl;
m is an integer of from 1 to 2;
n is an integer of from 1 to 3; and
optionally, a 5-, 6-, 7- or 8-member ring is formed by joining R¹ to R², R¹ to R³, R³ to N³, R⁵ to N³, R⁵ to N¹, or R² to N¹;
with the proviso that when Y is a bond, then R⁵ is other than an imidazole ring.

32. The method of claim 31, wherein R¹ is selected from the group consisting of —NO₂, —S(O)ₘNR⁷R⁸, —S(O)ₙR⁹, —CN, fluoroalkyl, —C(O)R⁹, —CO₂R¹⁰ and —C(O)NR⁷R⁸ and R² is selected from the group consisting of hydrogen, alkyl, —O-alkyl, —S-alkyl, aryl, arylalkyl, —O-aryl and —S-aryl.

33. The method of claim 31, wherein X is —NR³R⁴, Y is selected from the group consisting of —N(R⁶)—, —O— and —S—, R¹ is selected from the group consisting of —C(O)R⁹, —C(O)NR⁷R⁸, —S(O)ₙR⁹, —S(O)ₘNR⁷R⁸, —CO₂R¹⁰, —CN, fluoroalkyl and —NO₂, and R² is a member selected from the group consisting of hydrogen, alkyl, —O-alkyl and halogen.

34. The method of claim 31, wherein R¹ is selected from the group consisting of —CF₃, —S(O)ₘNR⁷R⁸, —CO₂R¹⁰, —CN and —NO₂, and R² is selected from the group consisting of hydrogen, (lower)alkyl, —O-(lower)alkyl and —S-(lower)alkyl.

35. The method of claim 31, wherein Y is —N(R⁶)— or —O—, R¹ is —NO₂, and R² is hydrogen or (C₁-C₄)alkyl.

36. The method of claim 31, wherein R³ is joined to R⁴ to form a 5-membered ring, together with the nitrogen to which both radicals are attached.

37. The method of claim 36, wherein said 5-membered ring contains two nitrogen atoms.

38. The method of claim 37, wherein said 5-membered ring is an imidazole ring.

39. The method of claim 31, wherein Y is —N(R⁶)—, in which R⁶ is hydrogen or lower alkyl, and R⁵ is a member selected from the group consisting of alkyl, aryl, arylalkyl and bicyclic fused aryl-cycloalkyl.

40. The method of claim 31, wherein R⁵ is selected from the group consisting of cycloalkyl, heterocycloalkyl, aryl, arylalkyl and bicyclic fused aryl-cycloalkyl, R⁶ is selected from the group consisting of hydrogen, methyl, ethyl and propyl, and —NR³R⁴ is selected from the group consisting of imidazol-1-yl, 2-methylimidazol-1yl, 2-ethylimidazol-1-yl, 2-(1-propyl)imidazol-1-yl and 2-(2-propyl)imidazol-1-yl.

41. The method of claim 31, wherein R⁶ is selected from the group consisting of hydrogen, methyl and ethyl, —NR³R⁴ is selected from the group consisting of imidazol-1-yl, 2-methylimidazol-1yl, 2,4-dimethylimidazol-1-yl and 2-ethylimidazol-1-yl, and R⁵ is an optionally substituted radical selected from the group consisting of

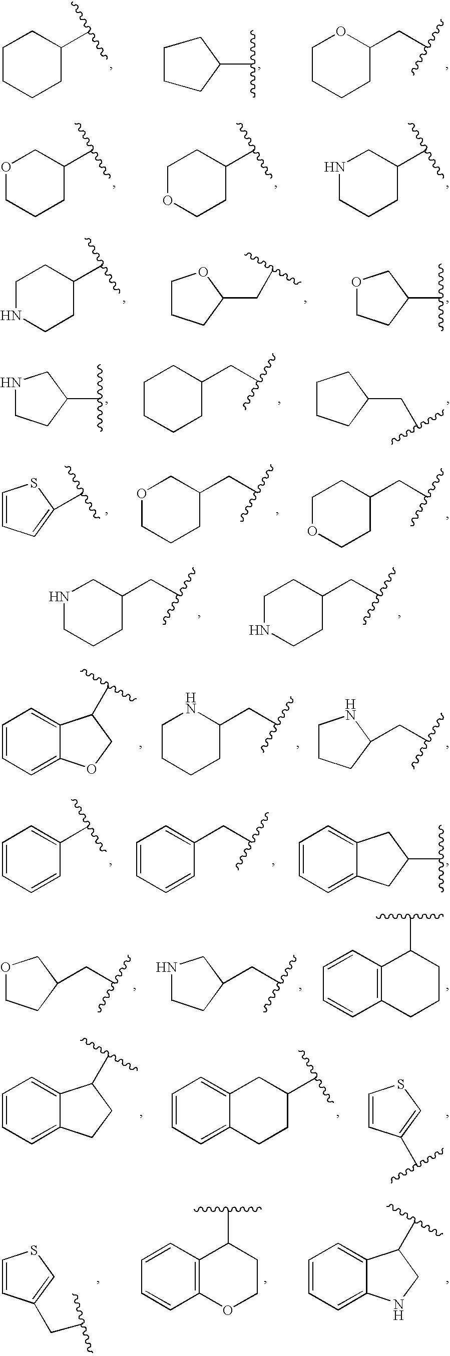

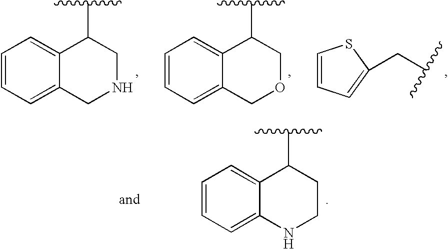

42. The method of claim 31, wherein R⁵ is a member selected from the group consisting of:

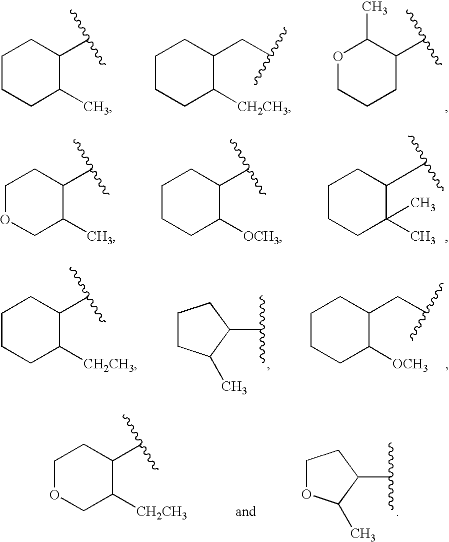

43. The method of claim 31, said compound having the formula: 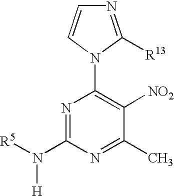
wherein
R¹³ is a member selected from the group consisting of hydrogen, methyl and ethyl; and
R⁵ is a member selected from the group consisting of:

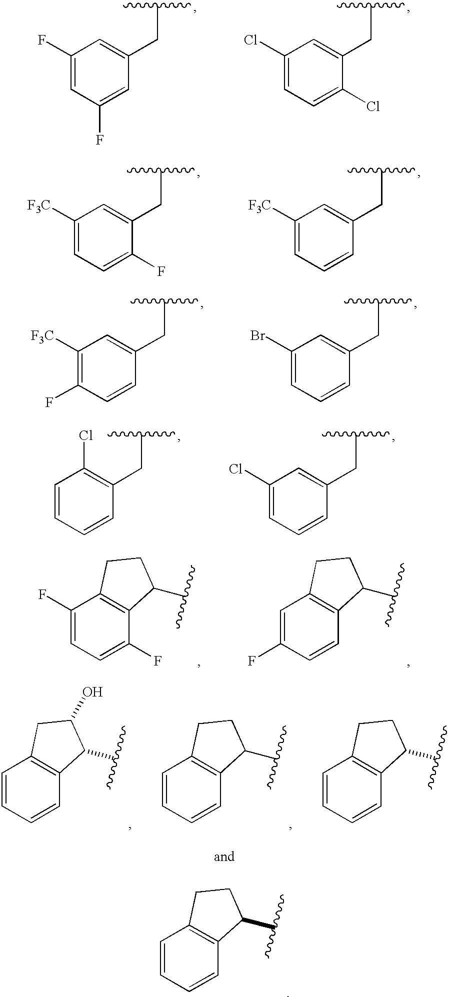

44. The method of claim 43, wherein R¹³ is methyl.

45. The method of claim 1, said compound having the formula: 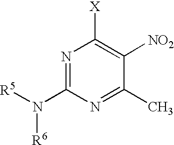
wherein
R¹³ is selected from the group consisting of hydrogen, methyl and ethyl; and
R⁵ and R⁶ are combined with the nitrogen atom to which R⁵ and R⁶ are attached to form a heterocycloalkyl ring.

46. The method of claim 45, said compound having the formula: 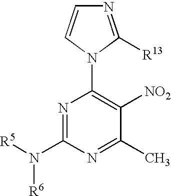
wherein
R¹³ is selected from the group consisting of hydrogen, methyl and ethyl; and
R⁵ and R⁶ are combined with the nitrogen atom to which R⁵ and R⁶ are attached to form a heterocycloalkyl ring.

47. The method of claim 46, wherein said heterocycloalkyl ring is selected from the group consisting of substituted or unsubstituted 1-piperidinyl, substituted or unsubstituted 4-morpholinyl and substituted or unsubstituted 1-pyrrolidinyl.

48. The method of claim 46 wherein R¹³ is hydrogen and R⁵ and R⁶ are combined with the nitrogen atom to which R⁵ and R⁶ are attached to form a substituted or unsubstituted 4-morpholinyl.

49. The method of claim 48, wherein R¹³ is hydrogen, R⁵ and R⁶ are combined with the nitrogen atom to which R⁵ and R⁶ are attached to form a monosubstituted 4-morpholinyl, said substituent being selected from the group consisting of (C₁-C₄)alkyl.

50. The method of claim 49, wherein said compound is selected from the group consisting of

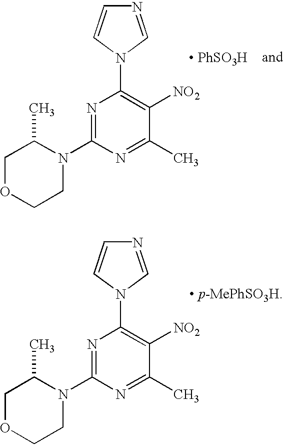

51. The method of claim 31, wherein said compound is administered in combination with a therapeutically effective amount of an agent selected from the group consisting of an antiviral agent, an agent used to treat atherosclerosis and an agent used to treat restenosis.

52. The method of claim 51, wherein said antiviral agent is selected from the group consisting of ganciclovir, valganciclovir, acyclovir, foscarnet, cidofovir and fomivirsen.

53. The method of claim 31, wherein said compound is administered in combination with an immunosuppressant agent.

54. The method of claim 31, wherein said disease is organ transplant rejection is selected from the group consisting of allograft rejection and xenograft rejection.

55. The method of claim 31, wherein said administering is oral.

56. The method of claim 31, wherein said administering is topical.

57. The method of claim 31, wherein said administering is parenteral.

58. The method of claim 31, wherein said disease is a cardiovascular disease selected from the group consisting of athersclerosis or restenosis.

59. The method of claim 31, wherein said disease is organ transplant-associated atherosclerosis.
